SEQUENCE LISTING (61 sequences)

SEQ ID NO: 1 - protein, 36 aa, Artificial Sequence
  source: /note=“Description of Artificial Sequence Synthetic polypeptide”
KGSKKAVTKAQKKDGKKRKRSRKESYSVYVYKVLKQ

SEQ ID NO: 2 - protein, 36 aa, Artificial Sequence
  source: /note=“Description of Artificial Sequence Synthetic polypeptide”
KGSKKAVTKAQKKEGKKRKRSRKESYSVYVYKVLKQ

SEQ ID NO: 3 - protein, 27 aa, Artificial Sequence
  source: /note=“Description of Artificial Sequence Synthetic peptide”
AQKKEGKKRKRSRKESYSVYVYKVLKQ

SEQ ID NO: 4 - protein, 18 aa, Artificial Sequence
  source: /note=“Description of Artificial Sequence Synthetic peptide”
KRSRKESYSVYVYKVLKQ

SEQ ID NO: 5 - protein, 18 aa, Artificial Sequence
  source: /note=“Description of Artificial Sequence Synthetic peptide”
KRSRKESYSVYVYKVLKQ

SEQ ID NO: 6 - protein, 13 aa, Artificial Sequence
  source: /note=“Description of Artificial Sequence Synthetic peptide”
ESYSVYVYKVLKQ

SEQ ID NO: 7 - protein, 6 aa, Artificial Sequence
  source: /note=“Description of Artificial Sequence Synthetic peptide”
YKVLKQ

SEQ ID NO: 8 - protein, 15 aa, Artificial Sequence
  source: /note=“Description of Artificial Sequence Synthetic peptide”
RVIRWFQNKRSKDKK

SEQ ID NO: 9 - protein, 27 aa, Artificial Sequence
  source: /note=“Description of Artificial Sequence Synthetic peptide”
GALFLGFLGAAGSTMGAWSQPKSKRKV

SEQ ID NO: 10 - protein, 16 aa, Artificial Sequence
  source: /note=“Description of Artificial Sequence Synthetic peptide”
RQIKIWFQNRRMKWKK

SEQ ID NO: 11 - protein, 16 aa, Artificial Sequence
  source: /note=“Description of Artificial Sequence Synthetic peptide”
RQIKIWFQNRRMKWKK

SEQ ID NO: 12 - protein, 26 aa, Artificial Sequence
  source: /note=“Description of Artificial Sequence Synthetic peptide”
GWTLNSAGYLLKINLKALAALAKKIL

SEQ ID NO: 13 - protein, 21 aa, Artificial Sequence
  source: /note=“Description of Artificial Sequence Synthetic peptide”
LLNQLAGRMIPKWSQKSKRKV

SEQ ID NO: 14 - protein, 20 aa, Artificial Sequence
  source: /note=“Description of Artificial Sequence Synthetic peptide”
TLDHVLDHVQTWSQKSKRKV

SEQ ID NO: 15 - protein, 23 aa, Artificial Sequence
  source: /note=“Description of Artificial Sequence Synthetic peptide”
SYFILRRRRKRFPYFFTDVRVAA

SEQ ID NO: 16 - protein, 10 aa, Artificial Sequence
  source: /note=“Description of Artificial Sequence Synthetic peptide”
RRRRRRRRRR

SEQ ID NO: 17 - protein, 8 aa, Artificial Sequence
  source: /note=“Description of Artificial Sequence Synthetic peptide”
RRRRRRRR

SEQ ID NO: 18 - protein, 28 aa, Artificial Sequence
  source: /note=“Description of Artificial Sequence Synthetic peptide”
KETWWETWWTEWSQPGRKKRRQRRRPPQ

SEQ ID NO: 19 - protein, 17 aa, Artificial Sequence
  source: /note=“Description of Artificial Sequence Synthetic peptide”
GRPRESGKKRKRKRLKP

SEQ ID NO: 20 - protein, 13 aa, Artificial Sequence
  source: /note=“Description of Artificial Sequence Synthetic peptide”
KSYSVYVYKVLKQ

SEQ ID NO: 21 - protein, 13 aa, Artificial Sequence
  source: /note=“Description of Artificial Sequence Synthetic peptide”
ESYSVYVYRVLRQ

SEQ ID NO: 22 - protein, 13 aa, Artificial Sequence
  source: /note=“Description of Artificial Sequence Synthetic peptide”
RSYSVYVYRVLRQ

SEQ ID NO: 23 - protein, 13 aa, Artificial Sequence
  source: /note=“Description of Artificial Sequence Synthetic peptide”
QKLVKYVYVSYSE

SEQ ID NO: 24 - protein, 13 aa, Artificial Sequence
  source: /note=“Description of Artificial Sequence Synthetic peptide”
ESYSVYVYKVLKQ

SEQ ID NO: 25 - protein, 13 aa, Artificial Sequence
  source: /note=“Description of Artificial Sequence Synthetic peptide”
ASYSVYVYAVLAQ

SEQ ID NO: 26 - protein, 13 aa, Artificial Sequence
  source: /note=“Description of Artificial Sequence Synthetic peptide”
QKLVKYVYVSYSE

SEQ ID NO: 27 - protein, 13 aa, Artificial Sequence
  source: /note=“Description of Artificial Sequence Synthetic peptide”
ESYSVYVYKVLKQ

SEQ ID NO: 28 - protein, 19 aa, Artificial Sequence
  source: /note=“Description of Artificial Sequence Synthetic peptide”
RRRRRRESYSVYVYKVLKQ

SEQ ID NO: 29 - protein, 19 aa, Artificial Sequence
  source: /note=“Description of Artificial Sequence Synthetic peptide”
ESYSVYVYKVLKQRRRRRR

SEQ ID NO: 30 - protein, 22 aa, Artificial Sequence
  source: /note=“Description of Artificial Sequence Synthetic peptide”
RRRRRRRQIKIWFQNRRMKWKK

SEQ ID NO: 31 - protein, 22 aa, Artificial Sequence
  source: /note=“Description of Artificial Sequence Synthetic peptide”
RQIKIWFQNRRMKWKKRRRRRR

SEQ ID NO: 32 - protein, 17 aa, Artificial Sequence
  source: /note=“Description of Artificial Sequence Synthetic peptide”
KTKIESLKEHGRRRRRR

SEQ ID NO: 33 - protein, 31 aa, Artificial Sequence
  source: /note=“Description of Artificial Sequence Synthetic polypeptide”
MDVNPTLLFLKVPAQNAISTTFPYTRRRRRR

SEQ ID NO: 34 - protein, 26 aa, Artificial Sequence
  source: /note=“Description of Artificial Sequence Synthetic peptide”
GLFEALLELLESLWELLLEARRRRRR

SEQ ID NO: 35 - protein, 18 aa, Artificial Sequence
  source: /note=“Description of Artificial Sequence Synthetic peptide”
LLNQLAGRMIPKRRRRRR

SEQ ID NO: 36 - protein, 17 aa, Artificial Sequence
  source: /note=“Description of Artificial Sequence Synthetic peptide”
TLDHVLDHVQTRRRRRR

SEQ ID NO: 37 - protein, 26 aa, Artificial Sequence
  source: /note=“Description of Artificial Sequence Synthetic peptide”
GLFGAIAGFIENGWEGMIDGRRRRRR

SEQ ID NO: 38 - protein, 17 aa, Artificial Sequence
  source: /note=“Description of Artificial Sequence Synthetic peptide”
KETWWETWWTERRRRRR

SEQ ID NO: 39 - protein, 16 aa, Artificial Sequence
  source: /note=“Description of Artificial Sequence Synthetic peptide”
HHHHHHHHHHRRRRRR

SEQ ID NO: 40 - protein, 22 aa, Artificial Sequence
  source: /note=“Description of Artificial Sequence Synthetic peptide”
AAVALLPAVLLALLAPRRRRRR

SEQ ID NO: 41 - protein, 18 aa, Artificial Sequence
  source: /note=“Description of Artificial Sequence Synthetic peptide”
KLALKLALKALKAALKLA

SEQ ID NO: 42 - DNA, 21 bp, Artificial Sequence
  source: /note=“Description of Combined DNA/RNA Molecule Synthetic oligonucleotide”
cuacacaaaucagcgauuutt

SEQ ID NO: 43 - DNA, 21 bp, Artificial Sequence
  source: /note=“Description of Combined DNA/RNA Molecule Synthetic oligonucleotide”
aaaucgcugauuuguguagtc

SEQ ID NO: 44 - RNA, 21 nt, Artificial Sequence
  source: /note=“Description of Artificial Sequence Synthetic oligonucleotide”
ggaaagacuguuccaaaaauu

SEQ ID NO: 45 - RNA, 21 nt, Artificial Sequence
  source: /note=“Description of Artificial Sequence Synthetic oligonucleotide”
uuuuuggaacagucuuuccuu

SEQ ID NO: 46 - protein, 7 aa, Artificial Sequence
  source: /note=“Description of Artificial Sequence Synthetic peptide”
KRRQRRR

SEQ ID NO: 47 - protein, 34 aa, Artificial Sequence
  source: /note=“Description of Artificial Sequence Synthetic polypeptide”
DAATATRGRSAASRPTERPRAPARSASRPRRPVD

SEQ ID NO: 48 - protein, 16 aa, Artificial Sequence
  source: /note=“Description of Artificial Sequence Synthetic peptide”
AAVALLPAVLLALLAP

SEQ ID NO: 49 - protein, 16 aa, Artificial Sequence
  source: /note=“Description of Artificial Sequence Synthetic peptide”
AAVLLPVLLPVLLAAP

SEQ ID NO: 50 - protein, 15 aa, Artificial Sequence
  source: /note=“Description of Artificial Sequence Synthetic peptide”
VTVLALGALAGVGVG

SEQ ID NO: 51 - protein, 17 aa, Artificial Sequence
  source: /note=“Description of Artificial Sequence Synthetic peptide”
GALFLGWLGAAGSTMGA

SEQ ID NO: 52 - protein, 17 aa, Artificial Sequence
  source: /note=“Description of Artificial Sequence Synthetic peptide”
MGLGLHLLVLAAALQGA

SEQ ID NO: 53 - protein, 24 aa, Artificial Sequence
  source: /note=“Description of Artificial Sequence Synthetic peptide”
LGTYTQDFNKFHTFPQTAIGVGAP

SEQ ID NO: 54 - protein, 16 aa, Artificial Sequence
  source: /note=“Description of Artificial Sequence Synthetic peptide”
TPPKKKRKVEDPKKKK

SEQ ID NO: 55 - protein, 16 aa, Artificial Sequence
  source: /note=“Description of Artificial Sequence Synthetic peptide”
GLFGAIAGFIENGWEG

SEQ ID NO: 56 - protein, 16 aa, Artificial Sequence
  source: /note=“Description of Artificial Sequence Synthetic peptide”
FFGAVIGTIALGVATA

SEQ ID NO: 57 - protein, 16 aa, Artificial Sequence
  source: /note=“Description of Artificial Sequence Synthetic peptide”
FLGFLLGVGSAIASGV

SEQ ID NO: 58 - protein, 16 aa, Artificial Sequence
  source: /note=“Description of Artificial Sequence Synthetic peptide”
GVFVLGFLGFLATAGS

SEQ ID NO: 59 - protein, 16 aa, Artificial Sequence
  source: /note=“Description of Artificial Sequence Synthetic peptide”
GAAIGLAWIPYFGPAA

SEQ ID NO: 60 - protein, 107 aa, Artificial Sequence
  source: /note=“Description of Artificial Sequence Synthetic polypeptide”
XXXXXXXXXXXXXXXXXXXXXXXXXXXXXXXXXXXXXXXXXXXXXXXXXXKRRQRRRXXXXXXXXXXXXXXXXXXXXXXXXXXXXXXXXXXXXXXXXXXXXXXXXXX

SEQ ID NO: 61 - protein, 50 aa, Artificial Sequence
  source: /note=“Description of Artificial Sequence Synthetic polypeptide”
RRRRRRRRRRRRRRRRRRRRRRRRRRRRRRRRRRRRRRRRRRRRRRRRRR

(19) United States
(12) Patent Application Publication
(10) Pub. No.: US 2011/0130327 A1
(43) Pub. Date: Jun. 2, 2011

(54) Multi-Arm Amines and Uses Thereof

(75) Inventors: MICHAEL E. HOUSTON, JR., SAMAMMISH, WA (US); ROGER C. ADAMI, BOTHELL, WA (US); STEVEN C. QUAY, SEATTLE, WA (US); PHUONG NGUYEN, CAMBRIDGE, MA (US); HARRY C. WANG, SAN DIEGO, CA (US); DIANE E. FRANK, SEATTLE, WA (US)

(73) Assignee: MARINA BIOTECH, INC., BOTHELL, WA (US)

(21) Appl. No.: 12/954,827

(22) Filed: Nov. 26, 2010

Related U.S. Application Data
(63) Continuation of application No. PCT/US2009/045664, filed on May 29, 2009.
(60) Provisional application No. 61/057,153, filed on May 29, 2008.

(51) Int. Cl. A61K 38/10; C07C 229/26; A61K 31/197; A61K 31/225; C07C 233/36; A61K 31/16; C07K 7/08; C07K 9/00; C07D 403/14; A61K 31/4178; C07D 403/12; A61K 31/7088; A61K 31/713; A61P 31/12; A61P 19/02; A61P 35/00; A61P 1/16; A61P 31/16; C12N 5/071

(52) U.S. Cl. 514/3.7; 562/564; 514/563; 560/169; 514/547; 564/153; 514/616; 530/327; 514/16.6; 514/4.3; 514/19.3; 514/21.5; 530/324; 514/21.3; 530/326; 548/312.7; 514/397; 514/44 R; 514/44 A; 435/325

(57) ABSTRACT

Multi-arm amine compounds and compositions for enhancing intracellular, in vitro, and in vivo delivery of drug, active, and therapeutic substances including ribonucleic acids. This disclosure provides novel compounds and compositions for making and using delivery materials and carriers which increase the efficiency of delivery of biologically active and pharmacologically active molecules. Embodiments of this disclosure may further provide delivery of various therapeutic agents including nucleic acid therapeutics such as regulatory RNA, interfering RNA, and agents for RNAi, as well as other protein and peptide therapeutics. In some aspects, this disclosure provides multi-arm amine compounds and compositions which can enhance permeation of a drug substance.

### CROSS REFERENCE TO RELATED APPLICATIONS

This application is a continuation of prior PCT International Application No. PCT/US2009/045664, filed May 29, 2009, which claims the benefit of U.S. Provisional Application No. 61/057,153, filed May 29, 2008, each of which is hereby incorporated by reference in its entirety.

### SEQUENCE LISTING

This application includes a Sequence Listing submitted herewith via EFS-Web as an ASCII file created on Nov. 15, 2010, named MAR210US1_SeqList.txt, which is 23,986 bytes in size, and is hereby incorporated by reference in its entirety.

### TECHNICAL FIELD

This disclosure relates to novel drug delivery-enhancing compounds and compositions useful for delivering various molecules and agents to cells, tissues, organs, and subjects. This disclosure provides a range of compounds, compositions, formulations, methods and uses of drug delivery enhancing compounds directed ultimately toward therapeutics and the diagnosis and treatment of diseases and conditions, including those that respond to modulation of gene expression or activity in a subject. More specifically, this disclosure relates to novel multi-arm amine compounds for delivery enhancing compositions and formulations, as well as therapeutic methods and uses thereof.

### BACKGROUND

The delivery of a therapeutic compound or material to a cell, organ or subject can be impeded by limited ability of the compound to reach a target cell or tissue and by restricted entry or trafficking of the compound within a cell. In general, delivery of a therapeutic material is restricted by the need to cross several cell membranes. Various mechanisms exist for selective entry of a compound into a cell while excluding exogenous molecules such as nucleic acids and proteins. These restrictions and barriers to delivery can result in the need for higher concentrations of a compound to be used to achieve a certain therapeutic result, which brings along the risk of higher toxicity and undesirable side effects.

One strategy to overcome delivery barriers is to improve the transport of a compound into cells by using lipid or polymeric carrier molecules. Carrier molecules can facilitate entry as a co-delivery enhancing agent, or as a conjugate partner which is covalently attached to facilitate delivery. Another strategy is to employ lipid molecules which can be organized into liposomes or other particles as carriers for drug agents. Liposomal drug carriers can improve uptake of a compound into cells, and can encapsulate or bind to a compound to cause transport of the compound across a cell membrane.

In these strategies, what is needed are lipophilic delivery-enhancing molecules which can interact with the compound or agent to be delivered in order to effect transport, while not impeding the release of the compound to reach its target.

The development of protein and peptide therapeutics, vaccine and antibody therapeutics, as well as nucleic acid therapeutics, among others, has increased the need for effective means of introducing active agents into cells, organs and subjects.

What is needed are compounds, compositions, methods and uses for improving cellular, systemic and local delivery of drug and biologically active substances in vivo, as well as in vitro.

### BRIEF SUMMARY

This disclosure provides novel drug delivery-enhancing multi-arm amine (MA) compounds, compositions and formulations for intracellular and in vivo delivery of drug agents for use, ultimately, as a therapeutic, which in general maintain cytoprotection and relatively low toxicity. The compounds and compositions of this disclosure are useful for delivery of drug agents to selected cells, tissues, organs or compartments in order to alter a disease state or a phenotype.

Among other things, this disclosure provides novel compounds and compositions for making and using delivery materials and carriers which increase the efficiency of delivery of biologically active and pharmacologically active molecules.

In some embodiments, the multi-arm amine compounds and compositions of this disclosure can enhance permeation of a drug substance in an epithelial layer, a mucosal tissue or layer, and in cells.

In some embodiments, the multi-arm amine compounds and compositions of this disclosure enhance the permeation of a drug substance across the blood-brain barrier.

The multi-arm amine compounds and compositions of this disclosure can be used for delivery of various therapeutic agents including nucleic acid therapeutics such as regulatory RNA, interfering RNA, and agents for RNAi, as well as protein and peptide therapeutics, vaccines, and antibody therapeutics. The multi-arm amine compounds and compositions of this disclosure can be used for delivery of a wide range of drug substances including biologically active agents and chemically active agents.

In some aspects, this disclosure provides compounds, compositions and methods to deliver a nucleic acid or RNA structure or construct to cells for regulating genomic expression or to produce the response of RNA interference. In some embodiments, this disclosure provides a range of novel multi-arm amine compounds for use as delivery agents for an interfering nucleic acid, or a precursor thereof, which may be employed in combination with other components including lipids, oils, emulsifiers, dispersants, and natural or synthetic polymeric materials.

In some embodiments, this disclosure includes multi-arm amine compounds having two or more tertiary amine groups substituted with three or more —CH₂(C═O)— groups, or substituted with three or more amide groups or ester groups.

In some embodiments, this disclosure includes multi-arm amine compounds having two or three tertiary amine groups, and containing three or more amide or ester groups.

In some aspects, the multi-arm amine compounds of this disclosure are composed of a core to which is attached a plurality of branched or unbranched arms. The core may be any polymer, chain, multimer, amine, or molecule having from one to five carboxylic acid groups attached. The arms may contain a lipophilic tail.

In certain embodiments, a multi-arm amine compound of this disclosure is an organic backbone or core, the backbone having from one to five —CH₂C(═O)R groups attached, wherein each —CH₂C(═O)R group is independently an ester or an amide group having from eight to twenty-two carbon atoms, as well as other variations having functional groups as disclosed below.

A multi-arm amine compound of this disclosure may have the structure shown in Formula I:

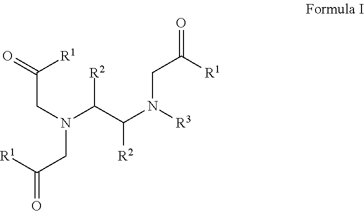

wherein
R¹ are independently, for each occurrence, selected from
   —OR⁴, —O(CH₂)ₙOR⁴, —O(CH₂CH₂O)ₗR⁴, —O(CH₂CH₂NH)ₘR⁴, —O(CH₂)ₙNR⁴R⁵;
   —NHR⁴, —NR⁴R⁵, —NH(CH₂CH₂O)ₗR⁴, —NH(CH₂CH₂NH)ₘR⁴, —NH(CH₂)ₙNR⁴R⁵, —NH(CH₂CH₂NH)ₘ(CH₂)ₙNR⁴R⁵, —NH(CH₂CH₂O)ₗ(CH₂)ₙNR⁴R⁵, —NH(CH₂CH₂O)ₗNR⁴R⁵; —NH(CH₂CH₂NH)ₘ(CH₂)ₙOR⁴;
   a nucleic acid;
   a peptide comprising 2-50 amino acid residues;
   a D- or L-amino acid residue;
   wherein
      R⁴ and R⁵ are independently, for each occurrence, hydrogen or a substituted or unsubstituted C(1-22)alkyl having 1, 2, 3, 4, 5, 6, 7, 8, 9, 10, 11, 12, 13, 14, 15, 16, 17, 18, 19, 20, 21, or 22 or C(2-22)alkenyl having 2, 3, 4, 5, 6, 7, 8, 9, 10, 11, 12, 13, 14, 15, 16, 17, 18, 19, 20, 21, or 22 carbon atoms;
      wherein R⁴ and R⁵ of —NR⁴R⁵ optionally form, together with the nitrogen atom of —NR⁴R⁵ to which they are attached, a saturated or unsaturated heterocyclic group optionally comprising one or more heteroatoms selected from oxygen, nitrogen and sulfur;
wherein one or more of the R¹ contains 4, 5, 6, 7, 8, 9, 10, 11, 12, 13, 14, 15, 16, 17, 18, 19, 20, 21, or 22 carbon atoms;
R² are independently, for each occurrence, selected from
   hydrogen, C(1-6)alkyl, —(CH₂)ₙaryl, —(CH₂)ₙ(C₆H₄)R⁶, —(CH₂)ₙ(C₆H₄)(CH₂)ₙNR⁴R⁵;
   wherein R⁶ is hydrogen, —OR⁴, —NR⁴R⁵; —O(CH₂)ₙOR⁴, —O(CH₂)ₙNR⁴R⁵, —(CH₂CH₂O)ₗR⁴; or —(CH₂CH₂NH)ₘR⁴;
R³ is selected from the group consisting of hydrogen, —(CH₂)ₙCOR¹, and

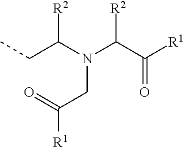

wherein
n is 1 to 22;
l is 1 to 50;
m is 1 to 50;
   and salts thereof.

In some embodiments, this disclosure includes multi-arm amine compounds wherein two or more of the R¹ contain 8, 9, 10, 11, 12, 13, 14, 15, 16, 17, 18, 19, 20, 21, or 22 carbon atoms.

In some embodiments, this disclosure includes multi-arm amine compounds wherein two of the R¹ contain 10 carbon atoms and the remaining R¹ are —OH, R² are hydrogen, and R³ is

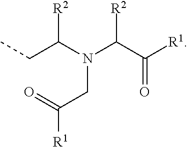

In some embodiments, this disclosure includes multi-arm amine compounds having the structure shown in Formula II:

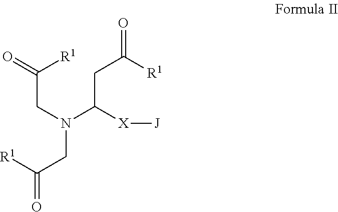

wherein
X is a linker;
J is —NH(C═O)R¹, —N(CH₂COR¹)₂, a nucleic acid, a D- or L-amino acid residue, a peptide having 2 to 50 amino acid residues; a protein, a sugar, or a vitamin;
R¹ are independently, for each occurrence, selected from the group consisting of
   —OR⁴, —O(CH₂)ₙOR⁴, —O(CH₂CH₂O)ₗR⁴, —O(CH₂CH₂NH)ₘR⁴, —O(CH₂)ₙNR⁴R⁵;
   —NHR⁴, —NR⁴R⁵, —NH(CH₂CH₂O)ₗR⁴, —NH(CH₂CH₂NH)ₘR⁴, —NH(CH₂)ₙNR⁴R⁵, —NH(CH₂CH₂NH)ₘ(CH₂)ₙNR⁴R⁵, —NH(CH₂CH₂O)ₗ(CH₂)ₙNR⁴R⁵, —NH(CH₂CH₂O)ₗNR⁴R⁵; —NH(CH₂CH₂NH)ₘ(CH₂)ₙOR⁴;
   wherein
      R⁴ and R⁵ are independently, for each occurrence, hydrogen or a substituted or unsubstituted C(1-22)alkyl having 1, 2, 3, 4, 5, 6, 7, 8, 9, 10, 11, 12, 13, 14, 15, 16, 17, 18, 19, 20, 21, or 22 carbon atoms or C(2-22)alkenyl having 2, 3, 4, 5, 6, 7, 8, 9, 10, 11, 12, 13, 14, 15, 16, 17, 18, 19, 20, 21, or 22 carbon atoms;
      wherein R⁴ and R⁵ of —NR⁴R⁵ optionally form, together with the nitrogen atom of —NR⁴R⁵ to which they are attached, a saturated or unsaturated heterocyclic group optionally comprising one or more heteroatoms selected from oxygen, nitrogen and sulfur;
         wherein
n is 1 to 22;
l is 1 to 50;
m is 1 to 50;
   and salts thereof.

In some embodiments, this disclosure includes multi-arm amine compounds wherein J is glutamate, aspartate, or cysteine.

In some embodiments, this disclosure includes multi-arm amine compounds wherein J is a peptide.

In some embodiments, this disclosure includes multi-arm amine compounds wherein J is mannose.

In some embodiments, this disclosure includes composition containing a multi-arm amine compound and a drug, a peptide drug, an RNA agent, or an interfering RNA agent.

In some embodiments, this disclosure includes methods for treating the signs and symptoms of inflammation or arthritis in a subject comprising administering to the subject in need a therapeutic amount of a composition containing a multi-arm amine compound and a drug, a peptide drug, an RNA agent, or an interfering RNA agent.

In some embodiments, this disclosure includes methods for treating the signs and symptoms of inflammation or arthritis in a subject comprising administering to the subject in need a therapeutic amount of a composition containing a multi-arm amine compound and a drug, a peptide drug, an RNA agent, or an interfering RNA agent. In some aspects, this disclosure includes pharmaceutical compositions containing an interfering RNA agent and a multi-arm amine compound.

In some embodiments, this disclosure includes methods for treating the signs and symptoms of cancer in a subject comprising administering to the subject in need a therapeutic amount of a composition containing a multi-arm amine compound and a drug, a peptide drug, an RNA agent, or an interfering RNA agent. In some aspects, this disclosure includes pharmaceutical compositions containing an interfering RNA agent and a multi-arm amine compound.

This summary, taken along with the detailed description of the disclosure, as well as the appended examples, claims, and drawings, if any, as a whole encompasses the disclosure of the disclosure.

### DETAILED DESCRIPTION

This disclosure provides novel multi-arm amine compounds, and compositions and formulations thereof, which have use for drug delivery methodologies including intracellular and in vivo delivery of drug and active agents. The compounds and compositions of this disclosure are useful for delivery of drug agents to cells, tissues, and subjects in order to ameliorate a condition or symptom, or alter a disease state or a phenotype.

This novel multi-arm amine compounds and compositions of this disclosure are sufficiently lipophilic to enhance delivery of a drug agent to cells or tissues, in vitro or in vivo.

In some embodiments, the multi-arm amine compounds and compositions of this disclosure can be used for delivery of nucleic acid therapeutics such as regulating RNA, interfering RNA, microRNA, antisense RNA, and small activating RNA, as well as protein and peptide therapeutics, vaccines, and monoclonal antibody therapeutics.

In some respects, this disclosure provides a range of compounds and compositions useful for delivering a nucleic acid or RNA structure or construct to cells to produce the response of RNA interference. The novel multi-arm amine compounds of this disclosure can be used as delivery agents for an interfering nucleic acid, or a precursor thereof, which may be employed in combination with other components including lipids, oils, emulsifiers, dispersants, and natural or synthetic polymeric materials.

In some aspects, the multi-arm amine compounds of this disclosure are composed of a core to which is attached a plurality of arms. The core may be any polymer, chain, multimer, amine, or molecule having from one to five carboxylic acid groups attached. The arms may be branched so that the number of arms may be from one to five, or more, depending on the degree of branching. The arms may be attached to the core through use of the carboxylic acid groups. In some variations, the arms may be attached to the core through the carboxylic acid groups to form an amide or ester group. The arms may contain various functional groups, including an alkyl, an amine, a polyethyleneglycol, an alkoxy, an amino acid, a peptide chain, a nucleic acid, a nucleic acid strand, and a double-stranded nucleic acid. These functional groups may be found in lengthy chains, or branched chains.

In certain variations, a multi-arm amine compound of this disclosure is an organic backbone or core consisting of from eight to fifty atoms selected from hydrogen, carbon, oxygen, nitrogen, and sulfur, the backbone having from three to five —CH₂C(═O)R groups attached, wherein each —CH₂C(═O)R group is independently an ester or an amide group having from eight to twenty-two carbon atoms. Each —CH₂C(═O)R group may be an alkyl ester or an alkyl amide.

In some embodiments, the core of a multi-arm amine compound may be a diethylene triamine compound.

In some embodiments, this disclosure may further provide multi-arm amine compounds having two or three tertiary amine groups, and containing three or more amide or ester groups.

In some embodiments, the multi-arm amine compound may be a chelant, chelator, chelating agent or sequestering agent. In some embodiments, the multi-arm amine compound may bind or complex a metal ion. In some embodiments, the multi-arm-amine compound may not be a chelant, chelator chelating agent or sequestering agent. In some embodiments, the multi-arm amine compound may not bind or complex a metal ion.

In some embodiments, a multi-arm amine compound of this disclosure may have an organic backbone having from eight to fifty atoms and containing one or more amine groups, the organic backbone having from one to five —CH₂C(═O)R groups attached, wherein each R group is an —X((C6-22)alkyl) group each independently having from six to twenty-two carbon atoms, wherein X is O or NH. A multi-arm amine compound may have two —CH₂C(═O)R groups attached, or three —CH₂C(═O)R groups attached, or four —CH₂C(═O)R groups attached, or five —CH₂C(═O)R groups attached. Each R group may be a lipophilic tail.

Examples of an organic backbone include ethylene diamine, diethylene triamine, a polymethylene chain having an amine group, and an amine compound.

Embodiments of this disclosure may further provide a multi-arm amine compound wherein the organic backbone has one or more lipophilic tails attached.

A multi-arm amine compound may have each R group an independently selected —NH(C10-22)alkyl group. In some variations, each R group is independently an —O(C10-22)alkyl group.

In some embodiments, an R group may include a polyethylene glycol group or an alkylamine group.

In some embodiments, this disclosure provides compounds having the structure shown in Formula I:

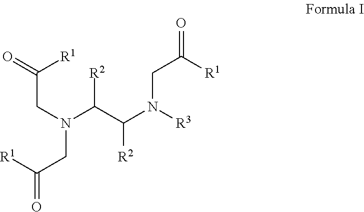

wherein
R¹ are independently, for each occurrence, selected from
   —OR⁴, —O(CH₂)ₙOR⁴, —O(CH₂CH₂O)ₗR⁴, —O(CH₂CH₂NH)ₘR⁴, —O(CH₂)ₙNR⁴R⁵;
   —NHR⁴, —NR⁴R⁵, —NH(CH₂CH₂O)ₗR⁴, —NH(CH₂CH₂NH)ₘR⁴, —NH(CH₂)ₙNR⁴R⁵, —NH(CH₂CH₂NH)ₘ(CH₂)ₙNR⁴R⁵, —NH(CH₂CH₂O)ₗ(CH₂)ₙNR⁴R⁵, —NH(CH₂CH₂O)ₗNR⁴R⁵; —NH(CH₂CH₂NH)ₘ(CH₂)ₙOR⁴;
   a nucleic acid;
   a peptide comprising 2-50 amino acid residues;
   a D- or L-amino acid residue having the formula —NR^{N}—CR⁸R⁹—(C═O)—NR⁴R⁵ or —NR^{N}—CR⁸R⁹—(C═O)—OH wherein
      R⁸ is independently, for each occurrence, a substituted or unsubstituted side chain of an amino acid;
      R⁹ is independently, for each occurrence, hydrogen, or an organic group consisting of carbon, oxygen, nitrogen, sulfur, and hydrogen atoms, and having from 1 to 20 carbon atoms, or C(1-5)alkyl, cycloalkyl, cycloalkylalkyl, C(3-5)alkenyl, C(3-5)alkynyl, C(1-5)alkanoyl, C(1-5)alkanoyloxy, C(1-5)alkoxy, C(1-5)alkoxy-C(1-5)alkyl, C(1-5)alkoxy-C(1-5)alkoxy, C(1-5)alkyl-amino-C(1-5)alkyl-, C(1-5)dialkyl-amino-C(1-5)alkyl-, nitro-C(1-5)alkyl, cyano-C(1-5)alkyl, aryl-C(1-5)alkyl, 4-biphenyl-C(1-5)alkyl, carboxyl, or hydroxyl,
      R^{N} is independently, for each occurrence, hydrogen, or an organic group consisting of carbon, oxygen, nitrogen, sulfur, and hydrogen atoms, and having from 1 to 20 carbon atoms, or C(1-5)alkyl, cycloalkyl, cycloalkylalkyl, C(3-5)alkenyl, C(3-5)alkynyl, C(1-5)alkanoyl, C(1-5)alkanoyloxy, C(1-5)alkoxy, C(1-5)alkoxy-C(1-5)alkyl, C(1-5)alkoxy-C(1-5)alkoxy, C(1-5)alkyl-amino-C(1-5)alkyl-, C(1-5)dialkyl-amino-C(1-5)alkyl-, nitro-C(1-5)alkyl, cyano-C(1-5)alkyl, aryl-C(1-5)alkyl, 4-biphenyl-C(1-5)alkyl, carboxyl, or hydroxyl;
   wherein
      R⁴ and R⁵ are independently, for each occurrence, hydrogen or a substituted or unsubstituted C(1-22)alkyl having 1, 2, 3, 4, 5, 6, 7, 8, 9, 10, 11, 12, 13, 14, 15, 16, 17, 18, 19, 20, 21, or 22 carbon atoms or C(2-22)alkenyl having 2, 3, 4, 5, 6, 7, 8, 9, 10, 11, 12, 13, 14, 15, 16, 17, 18, 19, 20, 21, or 22 carbon atoms;
      wherein R⁴ and R⁵ of —NR⁴R⁵ optionally form, together with the nitrogen atom of —NR⁴R⁵ to which they are attached, a saturated or unsaturated heterocyclic group optionally comprising one or more heteroatoms selected from oxygen, nitrogen and sulfur;
wherein one or more of the R¹ contains 4, 5, 6, 7, 8, 9, 10, 11, 12, 13, 14, 15, 16, 17, 18, 19, 20, 21, or 22 carbon atoms;
R² are independently, for each occurrence, selected from
   hydrogen, C(1-6)alkyl, —(CH₂)ₙaryl, —(CH₂)ₙ(C₆H₄)R⁶, —(CH₂)ₙ(C₆H₄)(CH₂)ₙNR⁴R⁵;
   wherein R⁶ is hydrogen, —OR⁴, —NR⁴R⁵; —O(CH₂)ₙOR⁴, —O(CH₂)ₙNR⁴R⁵, —(CH₂CH₂O)ₗR⁴; or —(CH₂CH₂NH)ₘR⁴;
R³ is selected from the group consisting of hydrogen, —(CH₂)ₙCOR¹, and

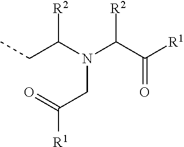

wherein
n is 1 to 22;
l is 1 to 50;
m is 1 to 50;
   and salts thereof.

In some embodiments, a compound of this disclosure may have two or more of the R¹ containing 8, 9, 10, 11, 12, 13, 14, 15, 16, 17, 18, 19, 20, 21, or 22 carbon atoms.

In some embodiments, a compound of this disclosure may have two of the R¹ containing 10 carbon atoms and the remaining R¹ are —OH, R² are hydrogen, and R³ is

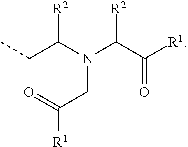

In some embodiments, a compound of this disclosure may have the structure shown in Formula I:

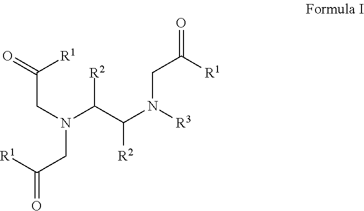

wherein
R¹ are independently, for each occurrence, selected from
   —NHR⁴, —NR⁴R⁵, —NH(CH₂CH₂O)ₗR⁴, —NH(CH₂CH₂NH)ₘR⁴, —NH(CH₂)ₙNR⁴R⁵, —NH(CH₂CH₂NH)ₘ(CH₂)ₙNR⁴R⁵, —NH(CH₂CH₂O)ₗ(CH₂)ₙNR⁴R⁵, —NH(CH₂CH₂O)ₗNR⁴R⁵; —NH(CH₂CH₂NH)ₘ(CH₂)ₙOR⁴;
   wherein
      R⁴ and R⁵ are independently, for each occurrence, hydrogen or a substituted or unsubstituted C(1-22)alkyl having 1, 2, 3, 4, 5, 6, 7, 8, 9, 10, 11, 12, 13, 14, 15, 16, 17, 18, 19, 20, 21, or 22 carbon atoms or C(2-22)alkenyl having 2, 3, 4, 5, 6, 7, 8, 9, 10, 11, 12, 13, 14, 15, 16, 17, 18, 19, 20, 21, or 22 carbon atoms;
      wherein R⁴ and R⁵ of —NR⁴R⁵ optionally form, together with the nitrogen atom of —NR⁴R⁵ to which they are attached, a saturated or unsaturated heterocyclic group optionally comprising one or more heteroatoms selected from oxygen, nitrogen and sulfur;
wherein one or more of the R¹ contains 4, 5, 6, 7, 8, 9, 10, 11, 12, 13, 14, 15, 16, 17, 18, 19, 20, 21, or 22 carbon atoms;
R² are independently, for each occurrence, selected from
   hydrogen, C(1-6)alkyl, —(CH₂)ₙaryl, —(CH₂)ₙ(C₆H₄)R⁶, —(CH₂)ₙ(C₆H₄)(CH₂)ₙNR⁴R⁵;
   wherein R⁶ is hydrogen, —OR⁴, —NR⁴R⁵; —O(CH₂)ₙOR⁴, —O(CH₂)ₙNR⁴R⁵, —(CH₂CH₂O)ₗR⁴; or —(CH₂CH₂NH)ₘR⁴;
R³ is selected from the group consisting of hydrogen, —(CH₂)ₙCOR¹, and

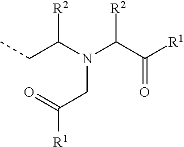

wherein
n is 1 to 22;
l is 1 to 50;
m is 1 to 50;
   and salts thereof.

In some embodiments, a compound of this disclosure may have the structure shown in Formula I:

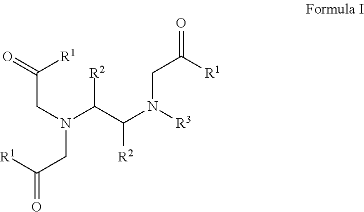

wherein
R¹ are independently, for each occurrence, selected from −OR⁴, —O(CH₂)ₙOR⁴, —O(CH₂CH₂O)ₗR⁴, —O(CH₂CH₂NH)ₘR⁴, —O(CH₂)ₙNR⁴R⁵;
   wherein
      R⁴ and R⁵ are independently, for each occurrence, hydrogen or a substituted or unsubstituted C(1-22)alkyl having 1, 2, 3, 4, 5, 6, 7, 8, 9, 10, 11, 12, 13, 14, 15, 16, 17, 18, 19, 20, 21, or 22 carbon atoms or C(2-22)alkenyl having 2, 3, 4, 5, 6, 7, 8, 9, 10, 11, 12, 13, 14, 15, 16, 17, 18, 19, 20, 21, or 22 carbon atoms;
      wherein R⁴ and R⁵ of —NR⁴R⁵ optionally form, together with the nitrogen atom of —NR⁴R⁵ to which they are attached, a saturated or unsaturated heterocyclic group optionally comprising one or more heteroatoms selected from oxygen, nitrogen and sulfur;
wherein one or more of the R¹ contains 4, 5, 6, 7, 8, 9, 10, 11, 12, 13, 14, 15, 16, 17, 18, 19, 20, 21, or 22 carbon atoms;
R² are independently, for each occurrence, selected from
   hydrogen, C(1-6)alkyl, —(CH₂)ₙaryl, —(CH₂)ₙ(C₆H₄)R⁶, —(CH₂)ₙ(C₆H₄)(CH₂)ₘNR⁴R⁵;
   wherein R⁶ is hydrogen, —OR⁴, —NR⁴R⁵; —O(CH₂)ₙOR⁴, —O(CH₂)ₙNR⁴R⁵, —(CH₂CH₂O)ₗR⁴; or —(CH₂CH₂NH)ₘR⁴;
R³ is selected from the group consisting of hydrogen, —(CH₂)ₙCOR¹, and

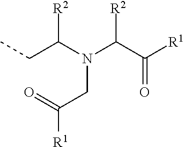

wherein
n is 1 to 22;
l is 1 to 50;
m is 1 to 50;
   and salts thereof.

In some embodiments, a compound of this disclosure may have the structure shown in Formula I:

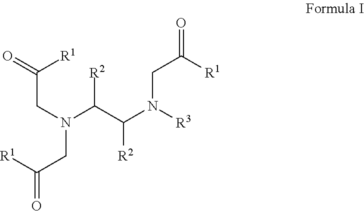

wherein
R¹ are independently, for each occurrence, selected from
   a peptide comprising 2-50 amino acid residues;
wherein one or more of the R¹ contains 4, 5, 6, 7, 8, 9, 10, 11, 12, 13, 14, 15, 16, 17, 18, 19, 20, 21, or 22 carbon atoms;
R² are independently, for each occurrence, selected from
   hydrogen, C(1-6)alkyl, —(CH₂)ₙaryl, —(CH₂)ₙ(C₆H₄)R⁶, —(CH₂)ₙ(C₆H₄)(CH₂)ₙNR⁴R⁵;
   wherein R⁶ is hydrogen, —OR⁴, —NR⁴R⁵; —O(CH₂)ₙOR⁴, —O(CH₂)ₙNR⁴R⁵, —(CH₂CH₂O)ₗR⁴; or —(CH₂CH₂NH)ₘR⁴;
   wherein R⁴ and R⁵ are independently, for each occurrence, hydrogen or a substituted or unsubstituted C(1-22)alkyl having 1, 2, 3, 4, 5, 6, 7, 8, 9, 10, 11, 12, 13, 14, 15, 16, 17, 18, 19, 20, 21, or 22 carbon atoms or C(2-22)alkenyl having 2, 3, 4, 5, 6, 7, 8, 9, 10, 11, 12, 13, 14, 15, 16, 17, 18, 19, 20, 21, or 22 carbon atoms;
   wherein R⁴ and R⁵ of —NR⁴R⁵ optionally form, together with the nitrogen atom of —NR⁴R⁵ to which they are attached, a saturated or unsaturated heterocyclic group optionally comprising one or more heteroatoms selected from oxygen, nitrogen and sulfur;
R³ is selected from the group consisting of hydrogen, —(CH₂)ₙCOR¹, and

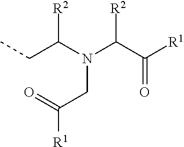

wherein
n is 1 to 22;
l is 1 to 50;
m is 1 to 50;
   and salts thereof.

In some embodiments, a compound of this disclosure may have the structure shown in Formula III:

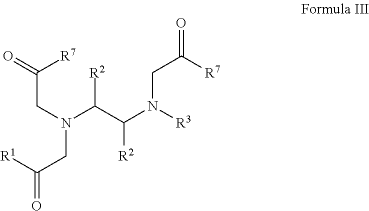

wherein
R¹ are independently, for each occurrence, selected from
   —OR⁴, —O(CH₂)ₙOR⁴, —O(CH₂CH₂O)ₗR⁴, —O(CH₂CH₂NH)ₘR⁴, —O(CH₂)ₙNR⁴R⁵;
   —NHR⁴, —NR⁴R⁵, —NH(CH₂CH₂O)ₗR⁴, —NH(CH₂CH₂NH)ₘR⁴, —NH(CH₂)ₙNR⁴R⁵, —NH(CH₂CH₂NH)ₘ(CH₂)ₙNR⁴R⁵, —NH(CH₂CH₂O)ₗ(CH₂)ₙNR⁴R⁵, —NH(CH₂CH₂O)ₗNR⁴R⁵; —NH(CH₂CH₂NH)ₘ(CH₂)ₙOR⁴;
R² are independently, for each occurrence, selected from
   hydrogen, C(1-6)alkyl, —(CH₂)ₙaryl, —(CH₂)ₙ(C₆H₄)R⁶, —(CH₂)ₙ(C₆H₄)(CH₂)ₙNR⁴R⁵;
   wherein R⁶ is hydrogen, —OR⁴, —NR⁴R⁵; —O(CH₂)ₙOR⁴, —O(CH₂)ₙNR⁴R⁵, —(CH₂CH₂O)ₗR⁴; or —(CH₂CH₂NH)ₘR⁴;
   wherein R⁴ and R⁵ are independently, for each occurrence, hydrogen or a substituted or unsubstituted C(1-22)alkyl having 1, 2, 3, 4, 5, 6, 7, 8, 9, 10, 11, 12, 13, 14, 15, 16, 17, 18, 19, 20, 21, or 22 carbon atoms or C(2-22)alkenyl having 2, 3, 4, 5, 6, 7, 8, 9, 10, 11, 12, 13, 14, 15, 16, 17, 18, 19, 20, 21, or 22 carbon atoms;
   wherein R⁴ and R⁵ of —NR⁴R⁵ optionally form, together with the nitrogen atom of —NR⁴R⁵ to which they are attached, a saturated or unsaturated heterocyclic group optionally comprising one or more heteroatoms selected from oxygen, nitrogen and sulfur;
R³ is selected from the group consisting of hydrogen, —(CH₂)ₙCOR¹, and

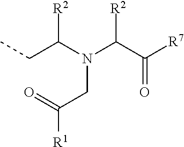

R⁷ is independently, for each occurrence, a D- or L-amino acid residue having the formula —NR^{N}—CR⁸R⁹—(C═O)—NR⁴R⁵ or —NR^{N}—CR⁸R⁹—(C═O)—OH, wherein
   R⁸ is independently, for each occurrence, a substituted or unsubstituted side chain of an amino acid;
   R⁹ is independently, for each occurrence, hydrogen, or an organic group consisting of carbon, oxygen, nitrogen, sulfur, and hydrogen atoms, and having from 1 to 20 carbon atoms, or C(1-5)alkyl, cycloalkyl, cycloalkylalkyl, C(3-5)alkenyl, C(3-5)alkynyl, C(1-5)alkanoyl, C(1-5)alkanoyloxy, C(1-5)alkoxy, C(1-5)alkoxy-C(1-5)alkyl, C(1-5)alkoxy-C(1-5)alkoxy, C(1-5)alkyl-amino-C(1-5)alkyl-, C(1-5)dialkyl-amino-C(1-5)alkyl-, nitro-C(1-5)alkyl, cyano-C(1-5)alkyl, aryl-C(1-5)alkyl, 4-biphenyl-C(1-5)alkyl, carboxyl, or hydroxyl,
   R^{N} is independently, for each occurrence, hydrogen, or an organic group consisting of carbon, oxygen, nitrogen, sulfur, and hydrogen atoms, and having from 1 to 20 carbon atoms, or C(1-5)alkyl, cycloalkyl, cycloalkylalkyl, C(3-5)alkenyl, C(3-5)alkynyl, C(1-5)alkanoyl, C(1-5)alkanoyloxy, C(1-5)alkoxy, C(1-5)alkoxy-C(1-5)alkyl, C(1-5)alkoxy-C(1-5)alkoxy, C(1-5)alkyl-amino-C(1-5)alkyl-, C(1-5)dialkyl-amino-C(1-5)alkyl-, nitro-C(1-5)alkyl, cyano-C(1-5)alkyl, aryl-C(1-5)alkyl, 4-biphenyl-C(1-5)alkyl, carboxyl, or hydroxyl,
      wherein
n is 1 to 22;
l is 1 to 50;
m is 1 to 50;
   and salts thereof.

In some embodiments, a compound of this disclosure may have the structure shown in Formula I:

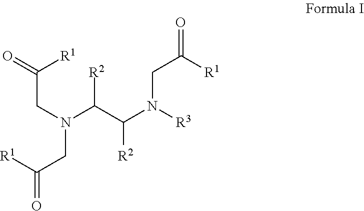

wherein
R¹ are independently, for each occurrence, selected from
   —NHR⁴, —NR⁴R⁵, —NH(CH₂CH₂O)ₘR⁴, —NH(CH₂CH₂NH)ₘR⁴, —NH(CH₂)ₙNR⁴R⁵, —NH(CH₂CH₂NH)ₘ(CH₂)ₙNR⁴R⁵, —NH(CH₂CH₂O)ₗ(CH₂)ₙNR⁴R⁵, —NH(CH₂CH₂O)ₗNR⁴R⁵; —NH(CH₂CH₂NH)ₘ(CH₂)ₙOR⁴;
   a nucleic acid;
   wherein
      R⁴ and R⁵ are independently, for each occurrence, hydrogen or a substituted or unsubstituted C(1-22)alkyl having 1, 2, 3, 4, 5, 6, 7, 8, 9, 10, 11, 12, 13, 14, 15, 16, 17, 18, 19, 20, 21, or 22 carbon atoms or C(2-22)alkenyl having 2, 3, 4, 5, 6, 7, 8, 9, 10, 11, 12, 13, 14, 15, 16, 17, 18, 19, 20, 21, or 22 carbon atoms;
      wherein R⁴ and R⁵ of —NR⁴R⁵ optionally form, together with the nitrogen atom of —NR⁴R⁵ to which they are attached, a saturated or unsaturated heterocyclic group optionally comprising one or more heteroatoms selected from oxygen, nitrogen and sulfur;
wherein one or more of the R¹ contains 4, 5, 6, 7, 8, 9, 10, 11, 12, 13, 14, 15, 16, 17, 18, 19, 20, 21, or 22 carbon atoms;
R² are independently, for each occurrence, selected from
   hydrogen, C(1-6)alkyl, —(CH₂)ₙaryl, —(CH₂)ₙ(C₆H₄)R⁶, —(CH₂)ₙ(C₆H₄)(CH₂)ₙNR⁴R⁵;
   wherein R⁶ is hydrogen, —OR⁴, —NR⁴R⁵; —O(CH₂)ₙOR⁴, —O(CH₂)ₙNR⁴R⁵, —(CH₂CH₂O)ₗR⁴; or —(CH₂CH₂NH)ₘR⁴;
R³ is selected from the group consisting of hydrogen, —(CH₂)ₙCOR¹, and

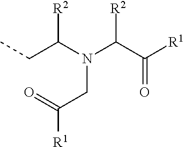

wherein
n is 1 to 22;
l is 1 to 50;
m is 1 to 50;
   and salts thereof.

Methods to prepare various organic groups and protective groups are known in the art and their use and modification is generally within the ability of one of skill in the art. See, e.g., Stanley R. Sandler and Wolf Karo, Organic Functional Group Preparations (1989); Greg T. Hermanson, Bioconjugate Techniques (1996); Leroy G. Wade, Compendium Of Organic Synthetic Methods (1980); examples of protective groups are found in T. W. Greene and P. G. M. Wuts, Protective Groups In Organic Synthesis (3rd ed. 1991).

In some embodiments, the multi-arm amine compound of this disclosure may contain from one to five (or 1, 2, 3, 4, 5) amino acid residue(s).

In some embodiments, the compound of this disclosure may contain an amino acid residue selected from arginine, homoarginine, norarginine, nor-norarginine, ornithine, lysine, homolysine, histidine, 1-methylhistidine, pyridylalanine, asparagine, N-ethylasparagine, glutamine, 4-aminophenylalanine, the N-methylated versions thereof, and side chain modified derivatives thereof. In some embodiments, the compound of this disclosure may contain an amino acid residue selected from cysteine and serine.

In certain embodiments, the amino acid residue may be a cationic amino acid residue, for example, where the amino acid residue has a basic side chain. Examples of amino acids having a basic side chain include arginine (Arg), homoarginine (homoArg) (side chain —(CH₂)₄NH(C═NH)NH₂), norarginine (norArg) (side chain —(CH₂)₂NH(C═NH)NH₂), nor-norarginine (nornorArg) (side chain —(CH₂)NH(C═NH)NH₂), ornithine, lysine, homolysine, histidine, 1-methylhistidine, pyridylalanine (Pal), asparagine, N-ethylasparagine, glutamine, and 4-aminophenylalanine, and side chain modified derivatives thereof.

As used herein, the term “homo” when referring to an amino acid, means that an additional carbon is added to the side chain, while the term “nor” when referring to an amino acid, means that a carbon is subtracted from the side chain. Thus, homolysine refers to side chain —(CH₂)₅NH₂.

In certain embodiments, the amino acid residue may be an anionic amino acid residue, for example, wherein the amino acid residue is glutamate or aspartate.

Compounds of this disclosure having cationic and anionic amino acid residues can also be prepared where the amino acid side chain contains an ionizable group or substituent.

Non-cationic amino acid residues may be leucine, valine, alanine, or serine.

In some embodiments, the amino acid residue is N^{G}-methylarginine, symmetric or asymmetric N^{G},N^{G}-dimethylarginine, N^{G}-methyl-homoarginine, symmetric or asymmetric N^{G},N^{G}-dimethyl-homoarginine, N^{G}-methyl-norarginine, symmetric or asymmetric N^{G},N^{G}-dimethyl-norarginine, or N^{G}-methyl-nor-norarginine, symmetric or asymmetric N^{G},N^{G}-dimethyl-nor-norarginine.

In some embodiments, the amino acid residue is N^{G}-ethylarginine, symmetric or asymmetric N^{G},N^{G}-diethylarginine, N^{G}-ethyl-homoarginine, symmetric or asymmetric N^{G},N^{G}-diethyl-homoarginine, N^{G}-ethyl-norarginine, symmetric or asymmetric N^{G},N^{G}-diethyl-norarginine, or N^{G}-ethyl-nor-norarginine, symmetric or asymmetric N^{G},N^{G}-diethyl-nor-norarginine.

In certain embodiments, the amino acid residue is N^{G}-alkylarginine, symmetric or asymmetric N^{G},N^{G}-dialkylarginine, N^{G}-alkyl-homoarginine, symmetric or asymmetric N^{G},N^{G}-dialkyl-homoarginine, N^{G}-alkyl-norarginine, symmetric or asymmetric N^{G},N^{G}-dialkyl-norarginine, or N^{G}-alkyl-nor-norarginine, symmetric or asymmetric N^{G},N^{G}-dialkyl-nor-norarginine.

In some embodiments, the amino acid residue is an amino acid having a guanidine- or amidine-containing side chain. For example, the side chain of the amino acid residue may contain a group such as guanido, amidino, dihydroimidazole, 4-guanido-phenyl, 4-amidino-phenyl, N-amidino-piperidine, N-amidino-piperazine, 4,5-dihydroimidazole, 2-(N-amidino)-pyrrolidinyl, or 4-[(2-aminopyrimidinyl)]ethyl.

Examples of amino acid residue side chains (e.g., R⁸ of Formula I, Formula II, or Formula III) include the following structures, as well as their salt forms:

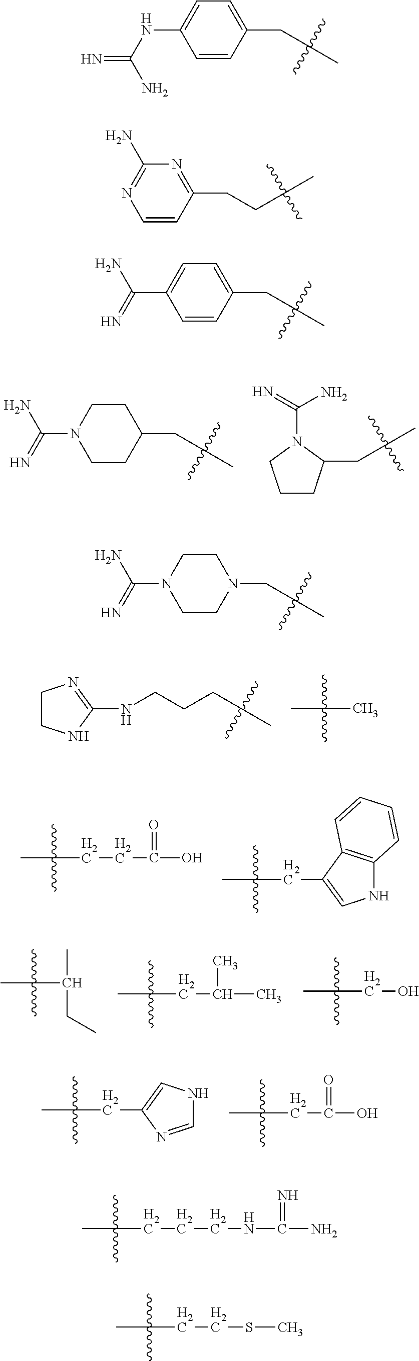

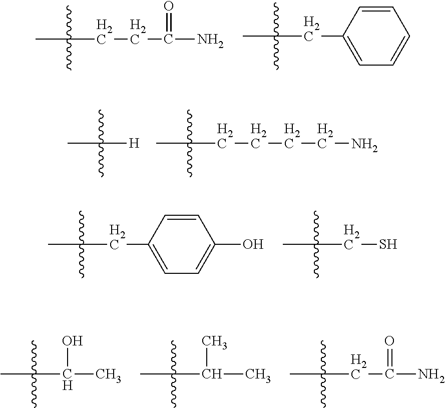

Examples of a substituted side chain of an amino acid suitable for a releasable form of an multi-arm amine compound include a releasing functional group having a pKa from about 5 to about 7.5, or from about 6 to about 7 (or 5, 5.5, 6, 6.5, 7, or 7.5). In general, a releasing functional group which is a weak base may exhibit a predominant neutral form at a local pH above pKa, and may exhibit a predominant ionic form at a local pH below pKa. A releasing functional group which is a weak acid may exhibit an ionic form at a local pH above pKa, and may exhibit a neutral form at a local pH below pKa. See, e.g., P. Heinrich Stahl, Handbook of Pharmaceutical Salts, (2002).

In some embodiments, the amino acid residue may have a side chain containing a functional group having a pKa from 5 to 7.5 (or 5, 5.5, 6, 6.5, 7, or 7.5).

Examples of a substituted side chain of an amino acid suitable for a releasable form of an multi-arm amine compound include 1-methylhistidine.

Examples of a substituted side chain of an amino acid suitable for a releasable form of an multi-arm amine compound include 3,5-diiodo-tyrosine.

Examples of a substituted side chain of an amino acid suitable for a releasable form of an multi-arm amine compound include the following structures:

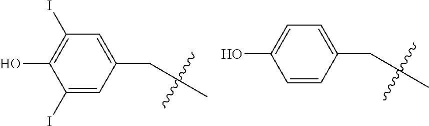

Examples of a substituent on a side chain of an amino acid suitable for a releasable form of a multi-arm amine compound include releasing functional groups derived from 3,5-diiodo-tyrosine, 1-methylhistidine, 2-methylbutanoic acid, 2-o-anisylpropanoic acid, meso-tartaric acid, 4,6-dimethylpyrimidinamine, p-phthalic acid, creatinine, butanoic acid, N,N-dimethyl-1-naphthylamine, pentanoic acid, 4-methylpentanoic acid, N-methylaniline, 1,10-phenanthroline, 3-pyridinecarboxylic acid, hexanoic acid, propanoic acid, 4-animobenzoic acid, 2-methylpropanoic acid, heptanoic acid, octanoic acid, cyclohexanecarboxylic acid, quinoline, 3-quinolinamine, 2-aminobenzoic acid, 4-pyridinecarboxylic acid, nonanic acid, melamine, 8-quinolinol, trimethylacetic acid, 6-methoxyquinoline, 4-(methylamino)benzoic acid, p-methylaniline, 3-(methylamino)benzoic acid, malic acid, N-ethylaniline, 2-benzylpyridine, 3,6-dinitrophenol, N,N-dimethylaniline, 2,5-dimethylpiperazine, p-phenetidine, 5-methylquinoline, 2-phenylbenzimidazole, pyridine, picolinic acid, 3,5-diiodityrosine, p-anisidine, 2-(methylamino)benzoic acid, 2-thiazolamine, glutaric acid, adipic acid, isoquinoline, itaconic acid, o-phthalic acid, benzimidazole, piperazine, heptanedioic acid, acridine, phenanthridine, succinic acid, methylsuccinic acid, 4-methylquinoline, 3-methylpyridine, 7-isoquinolinol, malonic acid, methymalonic acid, 2-methylquinoline, 2-ethylpyridine, 2-methylpyridine, 4-methylpyridine, histamine, histidine, maleic acid, cis-1,2-cyclohexanediamine, 3,5-dimethylpyridine, 2-ethylbenzimidazole, 2-methylbenzimidazole, cacodylic acid, perimidine, citric acid, isocitric acid, 2,5-dimethylpyridine, papaverine, 6-hydroxy-4-methylpteridine, L-thyroxine, 3,4-dimethylpyridine, methoxypyridine, trans-1,2-cyclohexanediamine, 2,5-pyridinediamine, l-1-methylhistidine, l-3-methylhistidine, 2,3-dimethylpyridine, xanthopterin, 1,2-propanediamine, N,N-diethylaniline, alloxanic acid, 2,6-dimethylpyridine, L-carnosine, 2-pyridinamine, N-b-alanylhistidine, pilocarpine, 1-methylimidazol, 1H-imidazole, 2,4-dimethylpyridine, 4-nitrophenol, 2-nitrophenol, tyrosineamide, 5-hydroxxyquinazoline, 1,1-cyclopropanedicarboxylic acid, 2,4,6-trimethylpyridine, veronal, 2,3-dichlorophenol, 1,2-ethanediamine, 1-isoquinolinamine, and combinations thereof.

In some embodiments, the sense strand of a dsRNA is linked to at least one compound of the disclosure. In some embodiments, the 5′-end of the sense strand of the dsRNA is linked to at least one compound of the disclosure. In some embodiments, the 3′-end of the sense strand of the dsRNA is linked to at least one compound of the disclosure. In some embodiments, the 5′-end of the sense strand of the dsRNA is linked to at least one compound of the disclosure, and the 3′-end of the sense strand of the dsRNA is linked to at least one compound of the disclosure.

In some embodiments, the antisense strand of the dsRNA is linked to at least one compound of the disclosure. In some embodiments, the 5′-end of the antisense strand of the dsRNA is linked to at least one compound of the disclosure. In some embodiments, the 3′-end of the antisense strand of the dsRNA is linked to at least one compound of the disclosure. In some embodiments, the 5′-end of the antisense strand of the dsRNA is linked to at least one compound of the disclosure, and the 3′-end of the antisense strand of the dsRNA is linked to at least one compound of the disclosure.

In some embodiments, the sense strand is linked to at least one compound of the disclosure and the antisense strand is linked to at least one compound of the disclosure. In some embodiments, the 5′-end of the sense strand of the dsRNA is linked to at least one compound of the disclosure and the 5′-end of the antisense strand of the dsRNA is linked to at least one compound of the disclosure. In some embodiments, the 5′-end of the sense strand of the dsRNA is linked to at least one compound of the disclosure and 3′-end of the antisense strand of the dsRNA is linked to at least one compound of the disclosure. In some embodiments, the 3′-end of the sense strand of the dsRNA is linked to at least one compound of the disclosure and the 5′-end of the antisense strand of the dsRNA is linked to at least one compound of the disclosure. In some embodiments, the 3′-end of the sense strand of the dsRNA is linked to at least one compound of the disclosure and the 3′-end of the antisense strand of the dsRNA is linked to at least one compound of the disclosure.

In some embodiments, the 5′-end of the sense strand of the dsRNA is linked to at least one compound of the disclosure, the 3′-end of the sense strand of the dsRNA is linked to at least one compound of the disclosure, the 5′-end of the antisense strand of the dsRNA is linked to at least one compound of the disclosure, and the 3′-end of the antisense strand of the dsRNA is linked to at least one compound of the disclosure.

In some embodiments, the 5′-end of an RNA is linked to at least one compound of the disclosure. In some embodiments, the 3′-end of an RNA is linked to at least one compound of the disclosure. In some embodiments, the 5′-end and the 3′-end of an RNA are linked to at least one compound of the disclosure.

In some embodiments, the 5′-end of a single stranded RNA is linked to at least one compound of the disclosure. In some embodiments, the 3′-end of a single stranded RNA is linked to at least one compound of the disclosure. In some embodiments, the 5′-end and the 3′-end of a single stranded RNA are linked to at least one compound of the disclosure.

As used herein, the term “linked” indicates a covalent bond between atoms of separate molecules (e.g., a covalent bond between an atom of an RNA molecule and an atom of a multi-arm amine compound).

In some embodiments, the RNA is linked directly to a multi-arm amine compound of this disclosure. In another embodiment, the RNA is linked to a multi-arm amine compound via a linker or linker group.

In some embodiments, a compound of this disclosure may have the structure shown in Formula II:

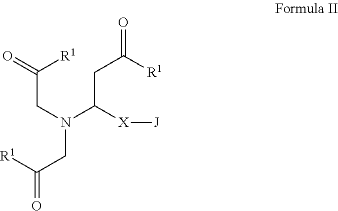

wherein
X is a linker to attach a nucleic acid, peptide, protein, sugar, or vitamin;
J is —NH(C═O)R¹, —N(CH₂COR¹)₂, a nucleic acid, a D- or L-amino acid residue, a peptide having 2 to 50 amino acid residues; a protein, a sugar, or a vitamin;
R¹ are independently, for each occurrence, selected from the group consisting of
   —OR⁴, —O(CH₂)ₙOR⁴, —O(CH₂CH₂O)ₗR⁴, —O(CH₂CH₂NH)ₘR⁴, —O(CH₂)ₙNR⁴R⁵;
   —NHR⁴, —NR⁴R⁵, —NH(CH₂CH₂O)ₗR⁴, —NH(CH₂CH₂NH)ₘR⁴, —NH(CH₂)ₙNR⁴R⁵, —NH(CH₂CH₂NH)ₘ(CH₂)ₙNR⁴R⁵, —NH(CH₂CH₂O)ₗ(CH₂)ₙNR⁴R⁵, —NH(CH₂CH₂O)ₗNR⁴R⁵; —NH(CH₂CH₂NH)ₘ(CH₂)ₙOR⁴;
   a peptide;
   a nucleic acid;
   a D- or L-amino acid residue having the formula —NR^{N}—CR⁸R⁹—(C═O)—NR⁴R⁵ or —NR^{N}—CR⁸R⁹—(C═O)—OH, wherein
      R⁸ is independently, for each occurrence, a substituted or unsubstituted side chain of an amino acid;
      R⁹ is independently, for each occurrence, hydrogen, or an organic group consisting of carbon, oxygen, nitrogen, sulfur, and hydrogen atoms, and having from 1 to 20 carbon atoms, or C(1-5)alkyl, cycloalkyl, cycloalkylalkyl, C(3-5)alkenyl, C(3-5)alkynyl, C(1-5)alkanoyl, C(1-5)alkanoyloxy, C(1-5)alkoxy, C(1-5)alkoxy-C(1-5)alkyl, C(1-5)alkoxy-C(1-5)alkoxy, C(1-5)alkyl-amino-C(1-5)alkyl-, C(1-5)dialkyl-amino-C(1-5)alkyl-, nitro-C(1-5)alkyl, cyano-C(1-5)alkyl, aryl-C(1-5)alkyl, 4-biphenyl-C(1-5)alkyl, carboxyl, or hydroxyl,
      R^{N} is independently, for each occurrence, hydrogen, or an organic group consisting of carbon, oxygen, nitrogen, sulfur, and hydrogen atoms, and having from 1 to 20 carbon atoms, or C(1-5)alkyl, cycloalkyl, cycloalkylalkyl, C(3-5)alkenyl, C(3-5)alkynyl, C(1-5)alkanoyl, C(1-5)alkanoyloxy, C(1-5)alkoxy, C(1-5)alkoxy-C(1-5)alkyl, C(1-5)alkoxy-C(1-5)alkoxy, C(1-5)alkyl-amino-C(1-5)alkyl-, C(1-5)dialkyl-amino-C(1-5)alkyl-, nitro-C(1-5)alkyl, cyano-C(1-5)alkyl, aryl-C(1-5)alkyl, 4-biphenyl-C(1-5)alkyl, carboxyl, or hydroxyl;
   wherein
      R⁴ and R⁵ are independently, for each occurrence, hydrogen or a substituted or unsubstituted C(1-22)alkyl having 1, 2, 3, 4, 5, 6, 7, 8, 9, 10, 11, 12, 13, 14, 15, 16, 17, 18, 19, 20, 21, or 22 carbon atoms, or C(2-22)alkenyl having 2, 3, 4, 5, 6, 7, 8, 9, 10, 11, 12, 13, 14, 15, 16, 17, 18, 19, 20, 21, or 22 carbon atoms;
      wherein R⁴ and R⁵ of —NR⁴R⁵ optionally form, together with the nitrogen atom of —NR⁴R⁵ to which they are attached, a saturated or unsaturated heterocyclic group optionally comprising one or more heteroatoms selected from oxygen, nitrogen and sulfur;
         wherein
n is 1 to 22;
l is 1 to 50;
m is 1 to 50;
   and salts thereof.

In some embodiments, the sense strand of the dsRNA is linked to at least one compound of the disclosure. In some embodiments, the 5′-end of the sense strand of the dsRNA is linked to at least one compound of the disclosure. In some embodiments, the 3′-end of the sense strand of the dsRNA is linked to at least one compound of the disclosure. In some embodiments, the 5′-end of the sense strand of the dsRNA is linked to at least one compound of the disclosure, and the 3′-end of the sense strand of the dsRNA is linked to at least one compound of the disclosure.

In some embodiments, the antisense strand of the dsRNA is linked to at least one compound of the disclosure. In some embodiments, the 5′-end of the antisense strand of the dsRNA is linked to at least one compound of the disclosure. In some embodiments, the 3′-end of the antisense strand of the dsRNA is linked to at least one compound of the disclosure. In some embodiments, the 5′-end of the antisense strand of the dsRNA is linked to at least one compound of the disclosure, and the 3′-end of the antisense strand of the dsRNA is linked to at least one compound of the disclosure.

In some embodiments, the sense strand is linked to at least one compound of the disclosure and the antisense strand is linked to at least one compound of the disclosure. In some embodiments, the 5′-end of the sense strand of the dsRNA is linked to at least one compound of the disclosure and the 5′-end of the antisense strand of the dsRNA is linked to at least one compound of the disclosure. In some embodiments, the 5′-end of the sense strand of the dsRNA is linked to at least one compound of the disclosure and 3′-end of the antisense strand of the dsRNA is linked to at least one compound of the disclosure. In some embodiments, the 3′-end of the sense strand of the dsRNA is linked to at least one compound of the disclosure and the 5′-end of the antisense strand of the dsRNA is linked to at least one compound of the disclosure. In some embodiments, the 3′-end of the sense strand of the dsRNA is linked to at least one compound of the disclosure and the 3′-end of the antisense strand of the dsRNA is linked to at least one compound of the disclosure.

In some embodiments, the 5′-end of the sense strand of the dsRNA is linked to at least one compound of the disclosure, the 3′-end of the sense strand of the dsRNA is linked to at least one compound of the disclosure, the 5′-end of the antisense strand of the dsRNA is linked to at least one compound of the disclosure, and the 3′-end of the antisense strand of the dsRNA is linked to at least one compound of the disclosure.

In some embodiments, the 5′-end of an RNA is linked to at least one compound of the disclosure. In some embodiments, the 3′-end of an RNA is linked to at least one compound of the disclosure. In some embodiments, the 5′-end and the 3′-end of an RNA are linked to at least one compound of the disclosure.

In some embodiments, the 5′-end of a single stranded RNA is linked to at least one compound of the disclosure. In some embodiments, the 3′-end of a single stranded RNA is linked to at least one compound of the disclosure. In some embodiments, the 5′-end and the 3′-end of a single stranded RNA are linked to at least one compound of the disclosure.

In some embodiments, J may be RNA, such as dsRNA, single stranded RNA, antisense RNA, microRNA, or shRNA.

In some embodiments, J may be glutamate, aspartate, or cysteine.

In some embodiments, J may be mannose or galactose.

This disclosure contemplates compositions containing a multi-arm amine compound and a drug, such as a peptide drug, an RNA agent, or an interfering RNA agent. Compositions may optionally contain one or more lipids.

This disclosure contemplates methods and uses for treating the signs and symptoms of inflammation, arthritis or cancer in a subject by administering to the subject in need a therapeutic amount of a composition containing a multi-arm amine compound and a drug.

In some embodiments, X is C(12-22)alkylene having 12, 13, 14, 15, 16, 17, 18, 19, 20, 21, or 22 carbon atoms and at least one of the R¹ contains at least 8, 9, 10, 11, 12, 13, 14, 15, 16, 17, 18, 19, 20, 21, or 22 carbon atoms.

In some embodiments, the peptide suitable for use as R¹ or J may be a cell penetrating peptide or fragment thereof, or a peptide selected from:

PN277
(SEQ ID NO: 1)
KGSKKAVTKAQKKDGKKRKRSRKESYSVYVYKVLKQ
PN857
(SEQ ID NO: 2)
KGSKKAVTKAQKKEGKKRKRSRKESYSVYVYKVLKQ
PN828
(SEQ ID NO: 3)
AQKKEGKKRKRSRKESYSVYVYKVLKQ
PN750
(SEQ ID NO: 4)
KRSRKESYSVYVYKVLKQ
PN872
(SEQ ID NO: 5)
PEG(200Da)-KRSRKESYSVYVYKVLKQ
PN963
(SEQ ID NO: 6)
ESYSVYVYKVLKQ
PN751
(SEQ ID NO: 7)
YKVLKQ
PN3072
(SEQ ID NO: 8)
RVIRWFQNKRSKDKK
PN3073
(SEQ ID NO: 9)
GALFLGFLGAAGSTMGAWSQPKSKRKV
PN3070
(SEQ ID NO: 10)
RQIKIWFQNRRMKWKK
PN3185
(SEQ ID NO: 11)
RQIKIWFQNRRMKWKK (all D-amino acids)
PN3071
(SEQ ID NO: 12)
GWTLNSAGYLLKINLKALAALAKKIL
PN3414
(SEQ ID NO: 13)
LLNQLAGRMIPKWSQKSKRKV
PN3415
(SEQ ID NO: 14)
TLDHVLDHVQTWSQKSKRKV
PN3416
(SEQ ID NO: 15)
SYFILRRRRKRFPYFFTDVRVAA
PN3079
(SEQ ID NO: 16)
RRRRRRRRRR
PN3671
(SEQ ID NO: 17)
RRRRRRRR (D-amino acids)
PN2986
(SEQ ID NO: 18)
KETWWETWWTEWSQPGRKKRRQRRRPPQ
PN740
(SEQ ID NO: 19)
GRPRESGKKRKRKRLKP
PN3846
(SEQ ID NO: 20)
KSYSVYVYKVLKQ
PN3847
(SEQ ID NO: 21)
ESYSVYVYRVLRQ
PN3848
(SEQ ID NO: 22)
RSYSVYVYRVLRQ
PN3884
(SEQ ID NO: 23)
QKLVKYVYVSYSE
PN3885
(SEQ ID NO: 24)
ESYSVYVYKVLKQ (all D amino acids)
PN3886
(SEQ ID NO: 25)
ASYSVYVYAVLAQ
PN3889
(SEQ ID NO: 26)
QKLVKYVYVSYSE (all D amino acids)
PN3948
(SEQ ID NO: 27)
ESYSVYVYKVLKQ-Peg(600Da)
PN3980
(SEQ ID NO: 28)
RRRRRRESYSVYVYKVLKQ
PN3981
(SEQ ID NO: 29)
ESYSVYVYKVLKQRRRRRR
PN3982
(SEQ ID NO: 30)
RRRRRRRQIKIWFQNRRMKWKK
PN3983
(SEQ ID NO: 31)
RQIKIWFQNRRMKWKKRRRRRR
PN3984
(SEQ ID NO: 32)
KTKIESLKEHGRRRRRR
PN3985
(SEQ ID NO: 33)
MDVNPTLLFLKVPAQNAISTTFPYTRRRRRR
PN3986
(SEQ ID NO: 34)
GLFEALLELLESLWELLLEARRRRRR
PN3987
(SEQ ID NO: 35)
LLNQLAGRMIPKRRRRRR
PN3988
(SEQ ID NO: 36)
TLDHVLDHVQTRRRRRR
PN3989
(SEQ ID NO: 37)
GLFGAIAGFIENGWEGMIDGRRRRRR
PN3990
(SEQ ID NO: 38)
KETWWETWWTERRRRRR
PN3991
(SEQ ID NO: 39)
HHHHHHHHHHRRRRRR
PN3992
(SEQ ID NO: 40)
AAVALLPAVLLALLAPRRRRRR
PN159
(SEQ ID NO: 41)
KLALKLALKALKAALKLA

The amino acid residues of the foregoing example peptides may be D or L in any frequency and order. It is understood by those of ordinary skill in the art that the amino acid sequence of the peptides of this disclosure may be represented by the standard one-letter or three-letter code as follows: aspartic acid (Asp) or D; glutamic acid (Glu) or E; lysine (Lys) or K; arginine (Arg) or R; histidine (His) or H; tyrosine (Tyr) or Y; cysteine (Cys) or C; asparagine (Asn) or N; glutamine (Gln) or Q; serine (Ser) or S; threonine (Thr) or T; glycine (Gly) or G; alanine (Ala) or A; valine (Val) or V; leucine (Leu) or L; isoleucine (Ile) or I; methionine (Met) or M; proline (Pro) or P; phenylalanine (Phe) or F; and tryptophan (Trp) or W.

A peptide of this disclosure may have from about two amino acid residues to about 500 amino acid residues; or from about two amino acid residues to about 400 amino acid residues; or from about two amino acid residues to about 300 amino acid residues; or from about two amino acid residues to about 200 amino acid residues; from about two amino acid residues to about 100 amino acid residues; or from about two amino acid residues to about 50 amino acid residues (or about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid residues).

A peptide of this disclosure may be amphipathic. A peptide of this disclosure may have one or more hydrophobic domains and one or more cationic domains. A hydrophobic domain may comprises a plurality of non-polar or hydrophobic amino acid residues (e.g., alanine, valine, leucine, isoleucine, proline, tyrosine, phenylalanine, tryptophan, methionine, cysteine or glycine). A cationic domain may comprise a plurality of charged amino acid residues (e.g., aspartic acid, glutamic acid, cystein, lysine, arginine, or histidine).

A peptide of this disclosure may be cationic having two or more cationic amino acid residues.

A peptide of this disclosure may be hydrophobic having two or more non-polar or hydrophobic amino acid residues.

A peptide of this disclosure may contain one or more domains containing an amino acid sequence from the following: KRRQRRR (SEQ ID NO:46); DAATATRGRSAASRPTERPRAPARSASRPRRPVD (SEQ ID NO:47); AAVALLPAVLLALLAP (SEQ ID NO:48); AAVLLPVLLPVLLAAP (SEQ ID NO:49); VTVLALGALAGVGVG (SEQ ID NO:50); GALFLGWLGAAGSTMGA (SEQ ID NO:51); MGLGLHLLVLAAALQGA (SEQ ID NO:52); LGTYTQDFNKFHTFPQTAIGVGAP (SEQ ID NO:53); TPPKKKRKVEDPKKKK (SEQ ID NO:54); (R)ₙ where R is arginine, and n is from 1 to 50; GLFGAIAGFIENGWEG (SEQ ID NO:55); FFGAVIGTIALGVATA (SEQ ID NO:56); FLGFLLGVGSAIASGV (SEQ ID NO:57); GVFVLGFLGFLATAGS (SEQ ID NO:58); GAAIGLAWIPYFGPAA (SEQ ID NO:59).

A peptide or peptide-containing composition of this disclosure can have a variant sequence that results from one or more conservative amino acid substitutions of the example peptides. The example peptides also include variants prepared by modifying the structure of a side chain of one or more amino acid residues. Variants of the example peptides may also have amino acid substitutions, deletions, insertions, or additions, wherein the variant sequence is at least 95% identical to the example peptide sequence. Information on amino acid substitutions is given in, for example, G. E. Schulz and R. H. Schirmer, Principles of Protein Structure (1979); C. Chothia and A. M. Lesk, 5 EMBO J. 823-26 (1986).

It is understood by those of ordinary skill in the art that an amino acid sequence can be modified by conservative amino acid substitutions while preserving functionality. In general, conservative replacements are permitted within a family of amino acids having related side chain structure. For example, replacement may be made amongst the (a) acidic residues aspartate and glutamate, (b) basic residues lysine, arginine, and histidine, (c) non-polar residues alanine, valine, leucine, isoleucine, proline, phenylalanine, methionine, and tryptophan; and (d) uncharged polar residues glycine, asparagine, glutamine, cysteine, serine, threonine, and tyrosine. Further, phenylalanine, tryptophan, and tyrosine may be considered a family of aromatic amino acids.

Examples of peptides suitable for use as R¹ or J are disclosed in U.S. Patent Publication No. 20060035815 A1.

Peptide compositions of this disclosure or variants thereof can be synthesized in vitro, e.g., by the solid phase peptide synthetic method or by enzyme catalyzed peptide synthesis or with the aid of recombinant DNA technology. Solid phase peptide synthetic method is an established and widely used method, which is described in references such as the following: Stewart et al., “Solid Phase Peptide Synthesis,” W.H. Freeman Co., San Francisco, 1969; Merrifield, J. Am. Chem. Soc. 85:2149, 1963; Meienhofer in “Hormonal Proteins and Peptides,” ed.; C. H. Li, Vol. 2, Academic Press, 1973, pp. 48-267; and Bavaay and Merrifield, “The Peptides,” eds. E. Gross and F. Meienhofer, Vol. 2, Academic Press, 1980, pp. 3-285. These peptides can be further purified by fractionation on immunoaffinity or ion-exchange columns; ethanol precipitation; reverse phase HPLC; chromatography on silica or on an anion-exchange resin such as DEAE; chromatofocusing; SDS-PAGE; ammonium sulfate precipitation; gel filtration using, for example, Sephadex G-75; ligand affinity chromatography; or crystallization or precipitation from non-polar solvent or nonpolar/polar solvent mixtures. Purification by crystallization or precipitation is available. Further methods are described in Stewart and Young, in “Solid Phase Peptide Synthesis”, 2nd ed., Pierce Chem. Co., Rockford, Ill., 1984; Wild et al., Proc. Natl. Acad. Sci. USA 89:10537, 1992; and Rimsky et al., J. Virol. 72:986, 1998.

A salt of a peptide or protein composition of this disclosure which is sufficiently basic may be an acid-addition salt with, for example, an inorganic or organic acid such as hydrochloric, hydrobromic, sulfuric, nitric, phosphoric, chlorosulfonic, trifluoroacetic, citric, maleic, acetic, propionic, oxalic, malic, maleic, malonic, fumaric, or tartaric acids, and alkane- or arenesulfonic acids such as methanesulfonic, ethanesulfonic, benzenesulfonic, chlorobenzenesulfonic, toluenesulfonic, naphthalenesulfonic, naphthalenedisulfonic, and camphorsulfonic acids.

A salt of a peptide or protein composition of this disclosure which is sufficiently acidic may be an alkali metal salt, for example, a sodium or potassium salt, or an alkaline earth metal salt, for example, a calcium or magnesium salt, or an ammonium salt or a salt with an organic base which provides a physiologically-acceptable cation, for example, a salt with methylamine, dimethylamine, trimethylamine, triethylamine, ethanolamine, diethanolamine, triethanolamine, ethylenediamine, tromethamine, N-methylglucamine, piperidine, morpholine or tris-(2-hydroxyethyl)amine See e.g., Berge et al., J. Pharm. Sci. 66:1, 1971.

The peptide or protein compositions of this disclosure may occur in the form of a pro-drug which may be an in vivo hydrolysable ester of a carboxy or hydroxy group of the peptide or protein, or in vivo hydrolysable amide of a carboxy group of the peptide or protein.

Some compounds, peptides and/or protein compositions of this disclosure may have one or more chiral centers and/or geometric isomeric centers (E- and Z-isomers), and it is to be understood that the disclosure encompasses all such optical isomers, diastereoisomers and geometric isomers.

This disclosure encompasses any and all tautomeric, solvated or unsolvated, and hydrated or unhydrated forms of the compounds, peptides and/or protein compositions disclosed herein.

This disclosure also contemplates compositions containing multi-arm amine compounds.

In some embodiments, a composition of this disclosure may contain a multi-arm amine compound and a drug or therapeutic substance. Examples of drug substances include biologically active agents and chemically active agents.

Examples of biologically active agents include nucleic acid therapeutics, regulatory RNA, interfering RNA, antisense compounds, oligonucleotides, and combinations thereof.

Examples of biologically active agents include proteins, polypeptides, peptides, hormones, vaccines, polysaccharides, carbohydrates, and lipids. Examples of biologically active agents include cytokines, colony stimulating factors such as GM-CSF, growth hormones, human growth hormones, recombinant human growth hormones, bovine growth hormones, porcine growth hormones, growth hormone-releasing hormones, interferons, alpha-interferon, beta-interferon, gamma-interferon, interleukin-1, interleukin-2, insulin, porcine insulin, bovine insulin, human insulin, human recombinant insulin, glucose regulating proteins, exendins, insulin-like growth factor, insulin-like growth factor-1, heparins, heparinoids, dermatans, chondroitins, calcitonin, salmon calcitonin, eel calcitonin, human calcitonin; erythropoietin (EPO), antigens, monoclonal antibodies, somatostatin, protease inhibitors, reverse transcriptase inhibitors, adrenocorticotropin, gonadotropin releasing hormone, oxytocin, carbetocin, leutinizing-hormone-releasing-hormone, follicle stimulating hormone, glucocerebrosidase, thrombopoietin, filgrastim, prostaglandins, cyclosporin, vasopressin, cromolyn sodium, sodium chromoglycate, disodium chromoglycate, vancomycin, desferrioxamine, neuropeptides, peptide YY, PYY, PYY(3-36), parathyroid hormone and fragments thereof, teriparatide, PTH(1-34), antimicrobials, anti-fungal agents, vitamins, and combinations thereof.

Examples of biologically active agents include therapeutic genes, vectors, plasmid vectors, viral vectors, antisense nucleic acids, triplex nucleic acids, therapeutic genes such as tumor suppressor genes, suicide genes, antisense nucleic acid molecules, triplex forming nucleic acid molecules, genes encoding cytokines, genes encoding Type I and Type II interferons such as interferon-alpha, interferon-beta, interferon-delta, and interferon-gamma, genes encoding interleukins including IL-1, IL-2, IL-4, Il-6, IL-7 and IL-10, and colony stimulating factors such as GM-CSF.

Examples of biologically active agents include naturally occurring or recombinantly modified substances.

Examples of chemically active agents include small molecule drugs and FDA-approved drugs. Examples of chemically active agents include anorexics, antiarthritics, antiasthmatic agents, anticonvulsants, antidepressants; antihistamines, anti-inflammatory agents, antinauseants, antineoplastics, antipruritics, antipsychotics, antipyretics, antispasmodics, cardiovascular preparations, antihypertensives, diuretics, vasodilators, central nervous system stimulants, cough and cold preparations, decongestants, diagnostics, hormones, bone growth stimulants and bone resorption inhibitors, immunosuppressives, muscle relaxants, psychostimulants, sedatives, tranquilizers, anti-inflammatory agents, anti-epileptics, anesthetics, hypnotics, sedatives, neuroleptic agents, antidepressants, anxiolytics, anticonvulsant agents, neuron blocking agents, anticholinergic and cholinomimetic agents, antimuscarinic and muscarinic agents, antiadrenergics, antiarrhythmics, antihypertensive agents, and combinations thereof.

In some embodiments, a composition of this disclosure may contain a multi-arm amine compound and a peptide drug.

In some embodiments, a composition of this disclosure may contain a multi-arm amine compound and an interfering RNA agent.

In some embodiments, a composition of this disclosure may contain a multi-arm amine compound an interfering RNA agent, and a lipid.

In some embodiments, a composition of this disclosure may contain a multi-arm amine compound wherein one of the R¹ is (C10)alkylene and the remaining R¹ are —OH, R² are hydrogen, R³ is

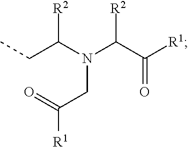

and an interfering RNA agent.

In some embodiments, a composition of this disclosure may contain a multi-arm amine compound wherein two of the R¹ are (C10)alkylene and the remaining R¹ are —OH, R² are hydrogen, R³ is

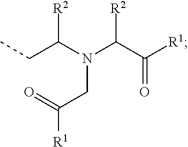

and an interfering RNA agent.

In some embodiments, a composition of this disclosure may contain a multi-arm amine compound wherein two of the R¹ are (C₁₋₆)alkylene and the remaining R¹ are —OH, R² are hydrogen, R³ is

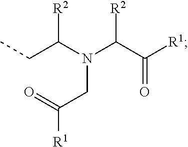

and an interfering RNA agent.

In some embodiments, a composition of this disclosure may contain a multi-arm amine compound wherein two of the R¹ are (C18)alkylene and the remaining R¹ are —OH, R² are hydrogen, R³ is

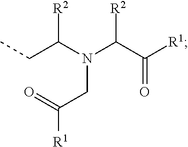

and an interfering RNA agent.

In some embodiments, a composition of this disclosure may contain a multi-arm amine compound wherein at least one of the R¹ is (C16)alkylene and at least one of the R¹ is (C18)alkylene and the remaining R¹ are —OH, R² are hydrogen, R³ is

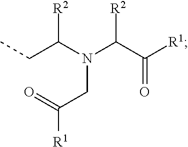

and an interfering RNA agent.

In some embodiments, R¹ may independently be C3alkyl, C4alkyl, C5alkyl, C6alkyl, C7alkyl, C8alkyl, C9alkyl, C10alkyl, C11alkyl, C12alkyl, C13alkyl, C14alkyl, C15alkyl, C16alkyl, C17alkyl, C18alkyl, C19alkyl, C20alkyl, C21alkyl, or C22alkyl.

In some embodiments, a first R¹ may be C3alkyl, C4alkyl, C5alkyl, C6alkyl, C7alkyl, C8alkyl, C9alkyl, C10alkyl, C11alkyl, C12alkyl, C13alkyl, C14alkyl, C15alkyl, C16alkyl, C17alkyl, C18alkyl, C19alkyl, C20alkyl, C21alkyl, or C22alkyl; and a second R¹ may be C3alkyl, C4alkyl, C5alkyl, C6alkyl, C7alkyl, C8alkyl, C9alkyl, C10alkyl, C11alkyl, C12alkyl, C13alkyl, C14alkyl, C15alkyl, C16alkyl, C17alkyl, C18alkyl, C19alkyl, C20alkyl, C21alkyl, or C22alkyl; where the first R¹ and the second R¹ have a different number of carbon atoms.

In some embodiments, R¹ may independently be lipophilic tails having one of the following structures:

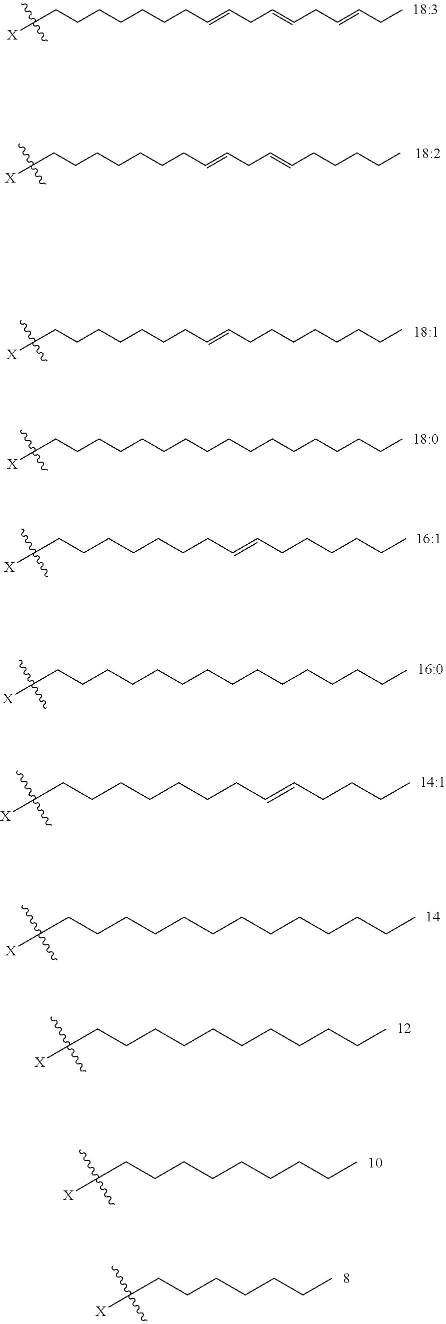

In the structures above, X represents the atom of the tail that is directly attached to the multi-arm amine compound and is counted as one of the atoms in the numerical designation, for example, “18:3.” In some embodiments, X may be a carbon, nitrogen, or oxygen atom.

In some embodiments, R¹ may independently be lipophilic tails having one of the following structures:

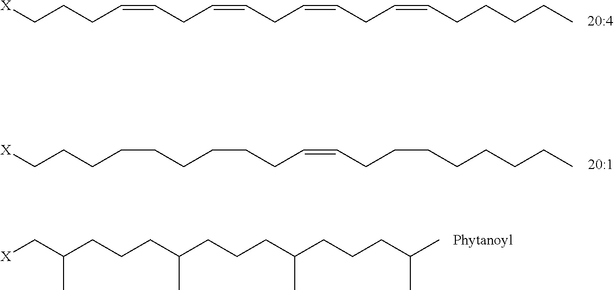

where X is as defined above.

In some embodiments, each R¹ of the multi-arm amine compound is a different lipophlic tail or has a different lipophilic length tail.

In some embodiment, at least two of the R¹ of the multi-arm amine compounds are asymmetric in length.

In some embodiments, R¹ is independently selected lipid-like tails which may contain a cholesterol, a sterol, or a steroid such as gonanes, estranes, androstanes, pregnanes, cholanes, cholestanes, ergostanes, campestanes, poriferastanes, stigmastanes, gorgostanes, lanostanes, cycloartanes, as well as sterol or zoosterol derivatives of any of the foregoing, and their biological intermediates and precursors, which may include, for example, cholesterol, lanosterol, stigmastanol, dihydrolanosterol, zymosterol, zymostenol, desmosterol, 7-dehydrocholesterol, and mixtures and derivatives thereof.

In certain embodiments, R¹ may independently be derived from fatty acid-like tails such as tails from myristic acid (C14:0)alkenyl, palmitic acid (C16:0)alkenyl, stearic acid (C18:0)alkenyl, oleic acid (C18:1, double bond at carbon 9)alkenyl, linoleic acid (C18:2, double bond at carbon 9 or 12)alkenyl, linonenic acid (C18:3, double bond at carbon 9, 12, or 15)alkenyl, arachidonic acid (C20:4, double bond at carbon 5, 8, 11, or 14)alkenyl, and eicosapentaenoic acid (C20:5, double bond at carbon 5, 8, 11, 14, or 17)alkenyl. Other examples of fatty acid-like tails are found at Donald Voet and Judith Voet, Biochemistry, 3rd Edition (2005), p. 383.

In some embodiments, R¹ may independently be derived from an isoprenoid. In some embodiments, R¹ may independently be derived from a naturally-occurring or synthetic lipid, phospholipid, glycolipid, triacylglycerol, glycerophospholipid, sphingolipid, ceramide, sphingomyelin, cerebroside, or ganglioside, wherein the tail may contain a steroid; or a substituted or unsubstituted C(3-22)alkyl, C(6-12)cycloalkyl, C(6-12)cycloalkyl-C(3-22)alkyl, C(3-22)alkenyl, C(3-22)alkynyl, C(3-22)alkoxy, or C(6-12)alkoxy-C(3-22)alkyl As used herein, the term “amino acid”, “amino acid residue” and “amino acid residue side chain” include naturally-occurring and non-naturally occurring amino acids. Thus, a multi-arm amine compound of this disclosure can be made from a genetically encoded amino acid, a naturally occurring non-genetically encoded amino acid, or a synthetic amino acid.

Examples of amino acids include azetidine, 2-aminooctadecanoic acid, 2-aminoadipic acid, 3-aminoadipic acid, 2,3-diaminopropionic acid, 2-aminobutyric acid, 4-aminobutyric acid, 2,3-diaminobutyric acid, 2,4-diaminobutyric acid, 2-aminoisobutyric acid, 4-aminoisobutyric acid, 2-aminopimelic acid, 2,2′-diaminopimelic acid, 6-aminohexanoic acid, 6-aminocaproic acid, 2-aminoheptanoic acid, desmosine, ornithine, citrulline, N-methylisoleucine, norleucine, tert-leucine, phenylglycine, t-butylglycine, N-methylglycine, sacrosine, N-ethylglycine, cyclohexylglycine, 4-oxo-cyclohexylglycine, N-ethylasparagine, cyclohexylalanine, t-butylalanine, naphthylalanine, pyridylalanine, 3-chloroalanine, 3-benzothienylalanine, 4-halophenylalanine, 4-chlorophenylalanine, 2-fluorophenylalanine, 3-fluorophenylalanine, 4-fluorophenylalanine, penicillamine, 2-thienylalanine, methionine, methionine sulfoxide, homoarginine, norarginine, nor-norarginine, N-acetyllysine, 4-aminophenylalanine, N-methylvaline, homocysteine, homoserine, hydroxylysine, allo-hydroxylysine, 3-hydroxyproline, 4-hydroxyproline, isodesmosine, allo-isoleucine, 6-N-methyllysine, norvaline, O-allyl-serine, O-allyl-threonine, alpha-aminohexanoic acid, alpha-aminovaleric acid, pyroglutamic acid, and derivatives thereof.

As used herein, the term “amino acid”, “amino acid residue” and “amino acid residue side chain” include alpha- and beta-amino acids.

Examples of amino acid residues can be found in Fasman, CRC Practical Handbook of Biochemistry and Molecular Biology, CRC Press, Inc. (1989).

In general, a compound may contain one or more chiral centers. Compounds containing one or more chiral centers may include those described as an “isomer,” a “stereoisomer,” a “diastereomer,” an “enantiomer,” an “optical isomer,” or as a “racemic mixture.” Conventions for stereochemical nomenclature, for example the stereoisomer naming rules of Cahn, Ingold and Prelog, as well as methods for the determination of stereochemistry and the separation of stereoisomers are known in the art. See, for example, Michael B. Smith and Jerry March, March's Advanced Organic Chemistry, 5th edition, 2001. The compounds and structures of this disclosure are meant to encompass all possible isomers, stereoisomers, diastereomers, enantiomers, and/or optical isomers that would be understood to exist for the specified compound or structure, including any mixture, racemic or otherwise, thereof.

The term “alkyl” as used herein refers to a saturated, branched or unbranched, substituted or unsubstituted aliphatic group containing from 1-22 carbon atoms. This definition applies to the alkyl portion of other groups such as, for example, alkoxy, alkanoyl, aralkyl, and other groups defined below. The term alkyl encompasses an aliphatic radical, while the term alkylene refers to an alkyl which is substituted in at least two positions. The term “cycloalkyl” as used herein refers to a saturated, substituted or unsubstituted cyclic alkyl ring containing from 3 to 12 carbon atoms. The term “alkenyl” as used herein refers to an unsaturated, branched or unbranched, substituted or unsubstituted alkyl or cycloalkyl having 2 to 22 carbon atoms and at least one carbon-carbon double bond. The term “alkynyl” as used herein refers to an unsaturated, branched or unbranched, substituted or unsubstituted alkyl or cycloalkyl having 2 to 22 carbon atoms and at least one carbon-carbon triple bond.

The term “alkoxy” as used herein refers to an alkyl, cycloalkyl, alkenyl, or alkynyl group covalently bonded to an oxygen atom. The term “alkanoyl” as used herein refers to —C(═O)-alkyl, which may alternatively be referred to as “acyl.” The term “alkanoyloxy” as used herein refers to —O—C(═O)-alkyl groups. The term “alkylamino” as used herein refers to the group —NRR′, where R and R′ are each either hydrogen or alkyl, and at least one of R and R′ is alkyl. Alkylamino includes groups such as piperidino wherein R and R′ form a ring. The term “alkylaminoalkyl” refers to -alkyl-NRR′.

The term “aryl” as used herein refers to any stable monocyclic, bicyclic, or polycyclic carbon ring system of from 4 to 12 atoms in each ring, wherein at least one ring is aromatic. Some examples of an aryl include phenyl, naphthyl, tetrahydro-naphthyl, indanyl, and biphenyl. Where an aryl substituent is bicyclic and one ring is non-aromatic, it is understood that attachment is to the aromatic ring. An aryl may be substituted or unsubstituted.

The term “heteroaryl” as used herein refers to any stable monocyclic, bicyclic, or polycyclic carbon ring system of from 4 to 12 atoms in each ring, wherein at least one ring is aromatic and contains from 1 to 4 heteroatoms selected from oxygen, nitrogen and sulfur. Some examples of a heteroaryl include acridinyl, quinoxalinyl, pyrazolyl, indolyl, benzotriazolyl, furanyl, thienyl, benzothienyl, benzofuranyl, quinolinyl, isoquinolinyl, oxazolyl, isoxazolyl, pyrazinyl, pyridazinyl, pyridinyl, pyrimidinyl, pyrrolyl, and tetrahydroquinolinyl. A heteroaryl includes the N-oxide derivative of a nitrogen-containing heteroaryl.

The term “heterocycle” or “heterocyclyl” as used herein refers to an aromatic or nonaromatic ring system of from five to twenty-two atoms, wherein from 1 to 4 of the ring atoms are heteroatoms selected from oxygen, nitrogen, and sulfur. Thus, a heterocycle may be a heteroaryl or a dihydro or tetrathydro version thereof.

The term “aroyl” as used herein refers to an aryl radical derived from an aromatic carboxylic acid, such as a substituted benzoic acid. The term “aralkyl” as used herein refers to an aryl group bonded to an alkyl group, for example, a benzyl group.

The term “carboxyl” as used herein represents a group of the formula —C(═O)OH or —C(═O)O⁻. The terms “carbonyl” and “acyl” as used herein refer to a group in which an oxygen atom is double-bonded to a carbon atom >C═O. The term “hydroxyl” as used herein refers to —OH or —O⁻. The term “nitrile” or “cyano” as used herein refers to —CN. The term “halogen” or “halo” refers to fluoro (—F), chloro (—Cl), bromo (—Br), and iodo (—I).

The term “substituted” as used herein refers to an atom having one or more substitutions or substituents which can be the same or different and may include a hydrogen substituent. Thus, the terms alkyl, cycloalkyl, alkenyl, alkynyl, alkoxy, alkanoyl, alkanoyloxy, alkylamino, alkylaminoalkyl, aryl, heteroaryl, heterocycle, aroyl, and aralkyl as used herein refer to groups which include substituted variations. Substituted variations include linear, branched, and cyclic variations, and groups having a substituent or substituents replacing one or more hydrogens attached to any carbon atom of the group. Substituents that may be attached to a carbon atom of the group include alkyl, cycloalkyl, alkenyl, alkynyl, alkoxy, alkanoyl, alkanoyloxy, alkylamino, alkylaminoalkyl, aryl, heteroaryl, heterocycle, aroyl, aralkyl, acyl, hydroxyl, cyano, halo, haloalkyl, amino, aminoacyl, alkylaminoacyl, acyloxy, aryloxy, aryloxyalkyl, mercapto, nitro, carbamyl, carbamoyl, and heterocycle. For example, the term ethyl includes without limitation —CH₂CH₃, —CHFCH₃, —CF₂CH₃, —CHFCH₂F, —CHFCHF₂, —CHFCF₃, —CF₂CH₂F, —CF₂CHF₂, —CF₂CF₃, and other variations as described above.

The term “linker group” or “linker” means an organic moiety that connects two parts of a compound (e.g., a multi-arm amine and a nucleic acid, peptide, sugar, and/or vitamin). A linker may contain atoms such as carbon, nitrogen, oxygen, and/or sulfur, a unit such as —NH—, —CH₂—, —C(O)—, —C(O)NH—, or a chain of atoms, such as an alkyl chain. Examples of linkers include a saturated or unsaturated C(1-22)alkyl having 1, 2, 3, 4, 5, 6, 7, 8, 9, 10, 11, 12, 13, 14, 15, 16, 17, 18, 19, 20, 21, or 22 carbon atoms or C(1-6)alkyl having 1, 2, 3, 4, 5, or 6 carbon atoms, which is optionally substituted, and wherein one or two saturated carbons of the chain are optionally replaced by —C(O)—, —C(O)C(O)—, —CONH—, —CONHNH—, —CO₂—, —OC(O)—, —NHCO₂—, —O—, —NHCONH—, —OC(O)NH—, —NHNH—, —NHCO—, —S—, —SO—, —SO₂—, —NH—, —SO₂NH—, or —NHSO₂—; and carbamates, maleimido, —NHS— amide, ester, or ether and derivatives thereof. Further, a linker or linker group may be cleavable by enzymes (e.g., intracellular enzymes) such as a disulfide or a Cathepsin B, D, or L substrate, or Val-Cit.

In certain embodiments, the linker is cleavable at low pH (e.g., below pH 7 or below pH 6, or below pH 5 or below pH 4). In other embodiments, the linker is cleaved in a cell organelle or compartment (e.g., an endosome).

As used herein, a chemical group described as having a range of atoms is to be understood as having any number of the atoms within the given range. For example, a C(1-22)alkyl group refers to an alkyl group having 1, 2, 3, 4, 5, 6, 7, 8, 9, 10, 11, 12, 13, 14, 15, 16, 17, 18, 19, 20, 21, or 22 carbon atoms. In another example, a C(1-22)alkylamino group refers to an alkylamino group having 1, 2, 3, 4, 5, 6, 7, 8, 9, 10, 11, 12, 13, 14, 15, 16, 17, 18, 19, 20, 21, or 22 carbon atoms.

In general, a compound may contain one or more chiral centers. Compounds containing one or more chiral centers may include those described as an “isomer,” a “stereoisomer,” a “diastereomer,” an “enantiomer,” an “optical isomer,” or as a “racemic mixture.” Conventions for stereochemical nomenclature, for example the stereoisomer naming rules of Cahn, Ingold and Prelog, as well as methods for the determination of stereochemistry and the separation of stereoisomers are known in the art. See, for example, Michael B. Smith and Jerry March “March's Advanced Organic Chemistry”, 5th edition, 2001. The compounds and structures of this disclosure are meant to encompass all possible isomers, stereoisomers, diastereomers, enantiomers, and/or optical isomers that would be understood to exist for the specified compound or structure, including any mixture, racemic or otherwise, thereof.

A pharmaceutically acceptable salt of a peptide or protein composition of this disclosure which is sufficiently basic may be an acid-addition salt with, for example, an inorganic or organic acid such as hydrochloric, hydrobromic, sulfuric, nitric, phosphoric, chlorosulfonic, trifluoroacetic, citric, maleic, acetic, propionic, oxalic, malic, maleic, malonic, fumaric, or tartaric acids, and alkane- or arenesulfonic acids such as methanesulfonic, ethanesulfonic, benzenesulfonic, chlorobenzenesulfonic, toluenesulfonic, naphthalenesulfonic, naphthalenedisulfonic, and camphorsulfonic acids.

A pharmaceutically acceptable salt of a peptide or protein composition of this disclosure which is sufficiently acidic may be an alkali metal salt, for example, a sodium or potassium salt, or an alkaline earth metal salt, for example, a calcium or magnesium salt, or an ammonium salt or a salt with an organic base which provides a physiologically-acceptable cation, for example, a salt with methylamine, dimethylamine, trimethylamine, triethylamine, ethanolamine, diethanolamine, triethanolamine, ethylenediamine, tromethamine, N-methylglucamine, piperidine, morpholine or tris-(2-hydroxyethyl)amine See, for example, Berge et al., J. Pharm. Sci. 66:1, 1971.

### Methods of Synthesis

The compounds of this disclosure may be manufactured by the methods provided below, by the methods provided in the examples or by analogous methods. Appropriate reaction conditions for the individual reaction steps are known to a person skilled in the art. Starting materials are either commercially available or can be prepared by methods analogous to the methods given below, by methods described in references cited in the text or in the examples, or by methods known in the art.

The methods below may serve as the basis for synthesizing various multi-amine compounds of this disclosure including monoalkylated, dialkylated, trialklated, tetraalkylated, and pentaalkylated. Multi-amine compounds of this disclosure may have homogenous or heterogenous aliphatic chain lengths; and have homogenous or hetergenous linkages (e.g., amide and ester linkages). Further, the multi-amine compounds of this disclosure may be alkylated and/or contain hydrophilic, cationic, neutral, zwitterionic functional groups; and nucleic acid, peptides, proteins, amino acid residues, and amino acid side chains.

Synthesis of monoalkylated multi-amine compounds (MA-(C1-22)monoalkyl) of this disclosure may be as follows:

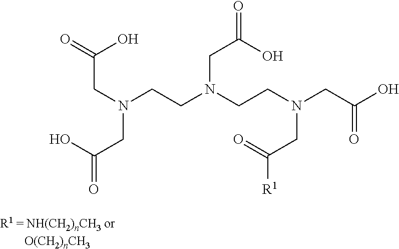

Monoalkylated amine compounds (as shown above) of this disclosure may be prepared as follows. The starting material diethylenetriaminepentaacetic acid dianhydride is suspended in dimethylformamide (DMF), or alternatively CH₂Cl₂, CHCl₃, or N-methylpyrrolidone (NMP); 4 eq of triethylamine (TEA), followed by ⅓ eq of CH₃(CH₂)ₙNH₂ amine and/or CH₃(CH₂)ₙOH (ester analogs; n dictates the length of the carbon tail attached). In the presence of DMF or NMP, after the reaction is complete, the reaction mixture is acidified to pH 2 with 1 M HCL. In the presence of CH₂Cl₂, CHCl₃, after the reaction is complete, solvent immiscible with water is removed under reduced pressure. Finally, residue is filtered off and washed with water and the crude is purified by RP-HPLC.

Synthesis of dialkylated multi-amine compounds (MA-(C1-22/C1-22)dialkyl) of this disclosure may be as follows:

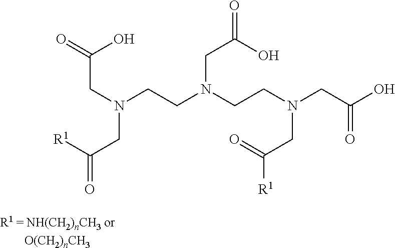

Dialkylated amine compounds (as shown above) of this disclosure may be prepared as follows. The starting material diethylenetriaminepentaacetic acid dianhydride is suspended in dimethylformamide (DMF), or alternatively CH₂Cl₂, CHCl₃, or N-methylpyrrolidone (NMP); 4 eq of triethylamine (TEA), followed by 3 eq of CH₃(CH₂)ₙNH₂ amine and/or CH₃(CH₂)ₙOH (ester analogs; n dictates the length of the carbon tail attached). In the presence of DMF or NMP, after the reaction is complete, the reaction mixture is acidified to pH 2 with 1 M HCL. In the presence of CH₂Cl₂, CHCl₃, after the reaction is complete, solvent immiscible with water is removed under reduced pressure. Finally, residue is filtered off and washed with water and the crude is purified by RP-HPLC or normal phase depending on aliphatic chain length (typically C10 and shorter lengths are purified by RP-HPLC and above C10 lengths are purified by normal phase).

Synthesis of a dialkylated multi-amine compounds having one or more functional groups (e.g., amino acid residue) of this disclosure may be as follows:

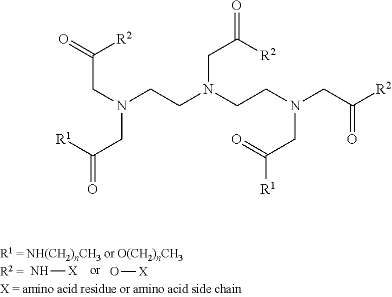

Continuing with the synthesis of the dialkylated amine compound show above, the MA-(C1-22/C1-22)dialkyl compound is dissolved in dimethylformamide (DMF) and 1.5 eq of a coupling regent (e.g., O-(6-chloro-1-hydrocibenzotriazol-1-yl)-1,1,3,3-tetramethyluronium hexafluorophosphate or 1-ethyl-3-[3-dimethylaminopropyl]carbodiimide hydrochloride) is added followed by 1.5 eq of amine (NH—X) or alcohol (O—X) based reagent, where X is, for example, an amino acid residue or a side chain of an amino acid residue. Protecting groups on the reactive moieties are added, if necessary. Lastly, 1.5 eq of N,N-diisopropylethylamine is added. After about 2 hours, the reaction mixture is diluted with water and the desired product is extracted three times with CH₂Cl₂. The organic layer is washed three times with a 1 M NaHSO₄, 1M NaHCO₃ aqueous solution followed by brine. The CH₂Cl₂ is dried with Mg(SO₄)₂ and is removed under reduced pressure. The crude is purified on normal phase silica gel.

Multi-are amine compounds of this disclosure may be covalently, either directly or indirectly via a linker, to one or more RNA molecules of this disclosure. In certain embodiments, the RNA molecule having an amino-terminated linker or TAP-(CH₂)ₙNH₂. In a subsequent operation, a multi-arm amine compound of this disclosure having an electrophilic group may subsequently be attached to the RNA molecule by coupling the electrophilic group of the multi-arm amine compound with a terminal nucleophilic group of the RNA molecule linker. Representative electrophilic groups include pentafluorophenyl esters or an aldehyde. Other electrophilic groups amenable to this method may be readily determined by one of ordinary skill in the art.

In certain embodiments, conjugation of the multi-arm amine compounds of this disclosure may be to an amino linker on an RNA molecule. For example, a multi-arm amine compound-RNA molecule conjugate of this disclosure may be prepared by conjugation of a multi-arm amine compound of this disclosure to an RNA molecule using EDC/sulfo-NHS (i.e., 1-ethyl-3-(3-dimethylaminopropylcarbodiimide/N-hydroxysulfosuccinimide) to conjugate the carboxylate function of the multi-arm amine compound with the amino function of the linking group on the RNA molecule.

In certain embodiments, the multi-arm amine compound-conjugated RNA molecule of this disclosure may be prepared by conjugation of multi-arm amine compound to the RNA molecule via a heterobifunctional linker such as m-maleimidobenzoyl-N-hydroxysulfosuccinimide ester (MBS) or succinimidyl 4-(N-maieimidomethyl)cyclo-hexane-1-carboxylate (SMCC), to link a nucleophilic position on the multi-arm amine compound to the amino function of the linker group on an RNA molecule. By this mechanism, an RNA-maleimide conjugate is formed by reaction of the amino group of the linker on the linked RNA molecule with the MBS or SMCC maleimide linker. The conjugate is then reacted with the multi-arm amine compound.

In other embodiments, a multi-arm amine compound conjugated-RNA molecule may be prepared by conjugation of the multi-arm amine compound to the RNA molecule via a homobifunctional linker such as disuccinimidyl suberate (DSS), to link an amino function on the multi-arm amine compound to the amino group of a linker on the RNA molecule. By this mechanism, an RNA-succinimidyl conjugate is formed by reaction of the amino group of the linker on the RNA with a disuccinimidyl suberate linker. The disuccinimidyl suberate linker couples with the amine linker on the RNA to extend the size of the linker. The extended linker is then reacted with an amino group of the multi-arm amine compound.

Synthesis of monoalkylated or dialkylated multi-arm amine compounds having one or more functional groups (e.g., nucleic acid molecule) of this disclosure may be performed by various synthesis reactions as provided below. The nucleic acid molecule may be a single-stranded RNA molecule, antisense RNA molecule double-stranded RNA molecule, siRNA, microRNA, shRNA, three-stranded RNA, or a combination of any of the above on a multi-arm amine compound.

Synthesis of a dialkylated multi-arm amine compound with attached RNA molecule(s) may be as follows:

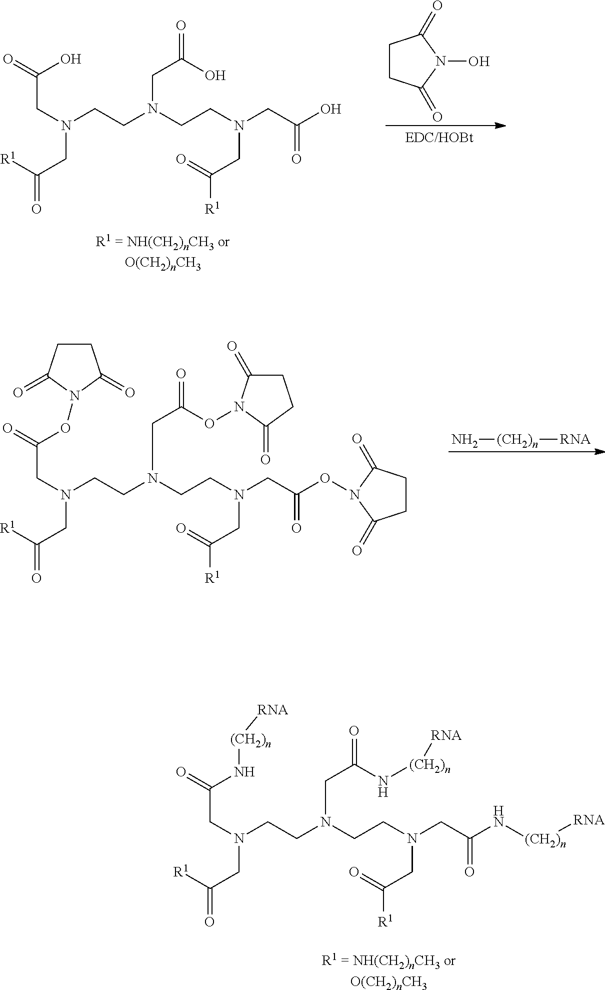

As shown in the schematic above, A MA-(C1-22/C1-22)dialkyl compound is dissolved in CH₂Cl₂ and 4 eq of NHS (N-hydroxysuccinimide) followed by 4 eq of EDC/HOBt and 3 eq of DIPEA. After the reaction is complete, the organic layer is washed three times with 1 M NaHCO₃ aqueous solution, water and followed by brine. The CH₂Cl₂ is dried with Mg(SO₄)₂ and is removed under reduced pressure. Crude NHS ester is used in a reaction with 4 eq of NH₂—(CH₂)ₙ—RNA molecule (n is from 1 to 22) in DMF/20 mM TRIS (tris(hydroxymethyl)aminomethane) buffer pH 7. In this embodiment, the linker is the alkyl chain —(CH₂)ₙ—, preferably where n is from 1 to 6. Desired product is purified by RP-HPLC. The formula of the product above shows three RNA molecules linked to the MA-(C1-22/C1-22)dialkyl compound; however, the synthesis above may result in a MA-(C1-22/C1-22)dialkyl compound having one, two, or three RNA molecules. These different compounds may be separated and isolated by RP-HPLC. Thus, one of ordinary skill in the art may synthesize and isolate the following structures:

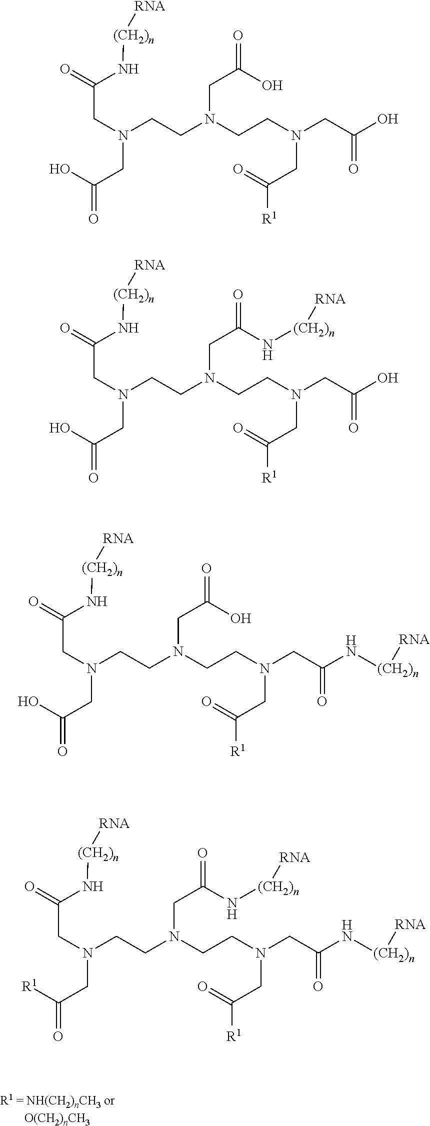

Synthesis of a trialkylated multi-arm amine compound with attached RNA molecule(s) may be as follows:

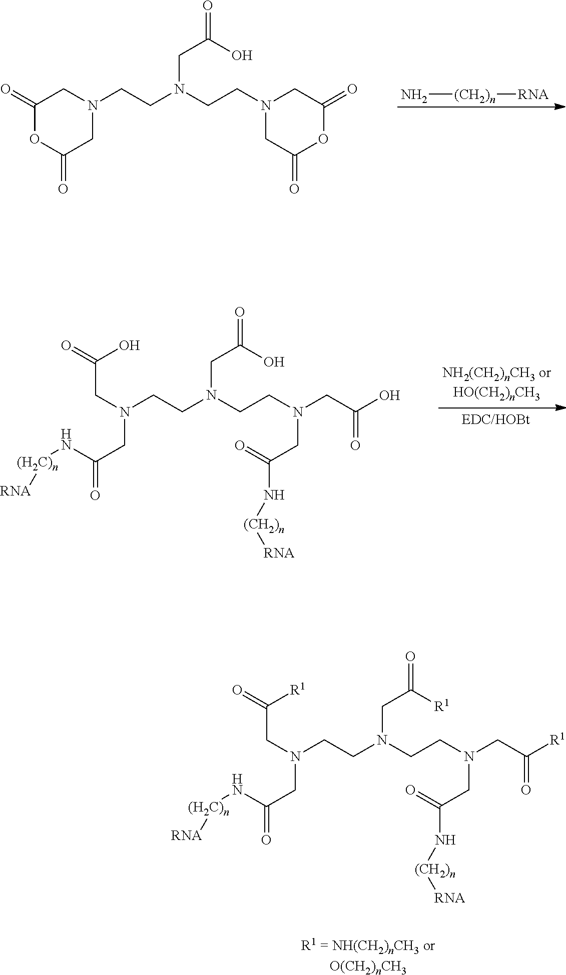

As shown in the schematic above, 2-(bis(2-(2,6-dioxomorpholino)ethyl)amino)acetic acid is dissolved in DMF/20 mM TRIS buffer pH 7 and 3 eq NH₂—(CH₂)ₙ-RNA molecule (n is from 1 to 22). In this embodiment, the linker is the alkyl chain —(CH₂)ₙ—, preferably where n is from 1 to 6. After the reaction is complete, the desired compound is purified by RP-HPLC. The purified intermediate is dissolved in dry dimethylformamide and 4 eq of coupling regent (EDC×HCL/HOBt) is added, followed by 4 eq of CH₃(CH₂)ₙNH₂ amine and/or CH₃(CH₂)ₙOH (ester analogs; n dictates the length of the carbon tail attached). Protecting groups on the reactive moieties are added, if necessary. Lastly, 4 eq of DIPEA is added. After two hours, the reaction mixture is diluted with water and the desired product is extracted three times with CH₂Cl₂. The CH₂Cl₂ is dried with Mg(SO₄)₂ and is removed under reduced pressure. The desired product may be purified by RP-HPLC.

Synthesis of a monoalkylated multi-arm amine compound with attached RNA molecule(s) is as follows:

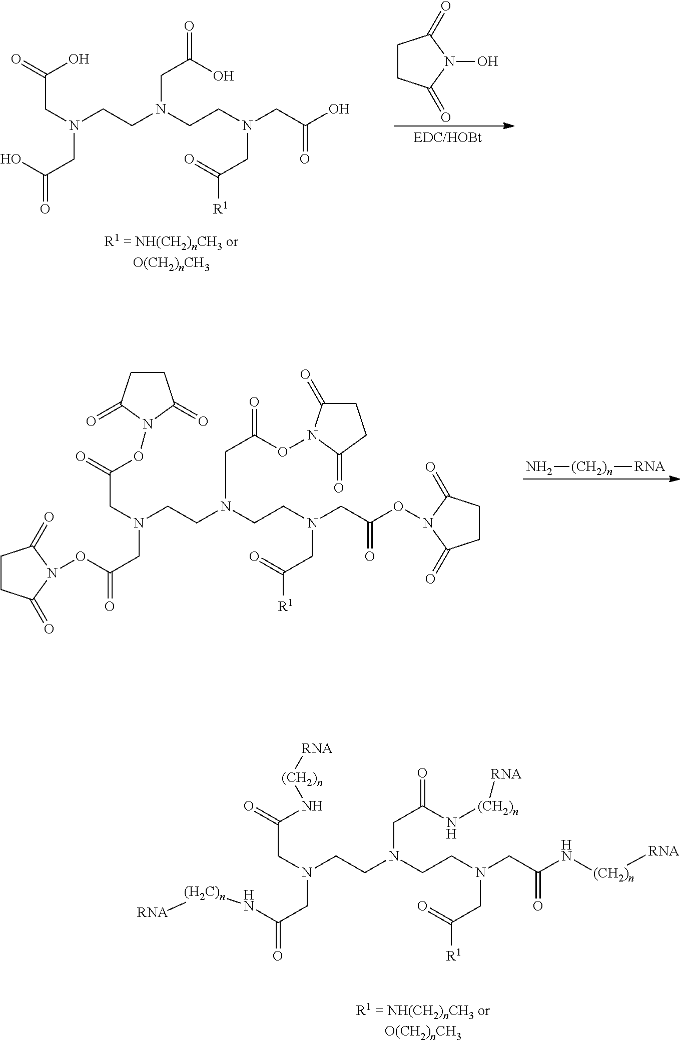

As shown in the schematic above, A MA-(C1-22/C1-22)dialkyl compound is dissolved in CH₂Cl₂ and 5 eq of NHS (N-hydroxysuccinimide) followed by 4 eq of EDC/HOBt and 3 eq of DIPEA. After the reaction is complete, the organic layer is washed three times with 1 M NaHCO₃ aqueous solution, water and followed by brine. The CH₂Cl₂ is dried with Mg(SO₄)₂ and is removed under reduced pressure. Crude NHS ester is used in a reaction with 5 eq of NH₂-Linker-(CH₂)ₙ-RNA molecule (n is from 0 to about 22) in DMF/20 mM TRIS (tris(hydroxymethyl)aminomethane) buffer pH 7. Desired product is purified by RP-HPLC. Likewise, the monoalkylated multi-arm amine compound may have one, two, three, or four RNA molecules. These different compounds may be separated and isolated by RP-HPLC.

Synthesis of a tetraalkylated multi-arm amine compound with attached RNA molecule(s) is as follows:

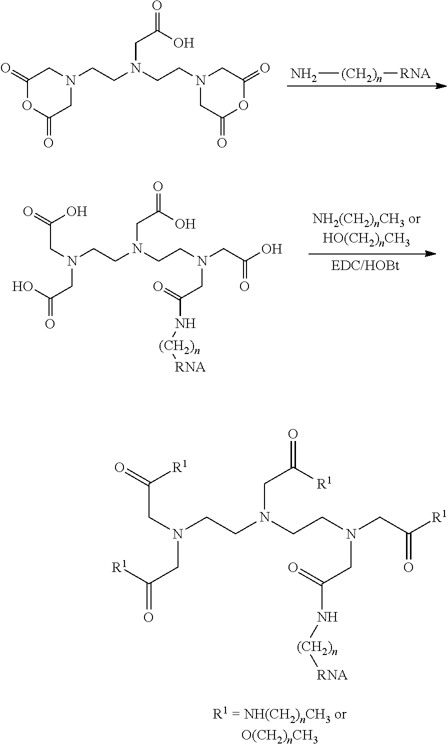

As shown in the schematic above, 2-(bis(2-(2,6-dioxomorpholino)ethyl)amino)acetic acid is dissolved in DMF/20 mM TRIS buffer pH 7 and ⅓ eq NH₂—(CH₂)ₙ-RNA molecule (n is from 1 to 22). In this embodiment, the linker is the alkyl chain —(CH₂)ₙ—, preferably where n is from 1 to 6. After the reaction is complete, the desired compound is purified by RP-HPLC. The purified intermediate is dissolved in dry dimethylformamide and 5 eq of coupling regent (EDC×HCL/HOBt) is added, followed by 4 eq of CH₃(CH₂)ₙNH₂ amine and/or CH₃(CH₂)ₙOH (ester analogs; n dictates the length of the carbon tail attached). Protecting groups on the reactive moieties are added, if necessary. Lastly, 4 eq of DIPEA is added. After two hours, the reaction mixture is diluted with water and the desired product is extracted three times with CH₂Cl₂. The CH₂Cl₂ is dried with Mg(SO₄)₂ and is removed under reduced pressure. The desired product may be purified by RP-HPLC.

Synthesis of a dialkylated multi-arm amine compound with attached RNA molecule(s) may be as follows:

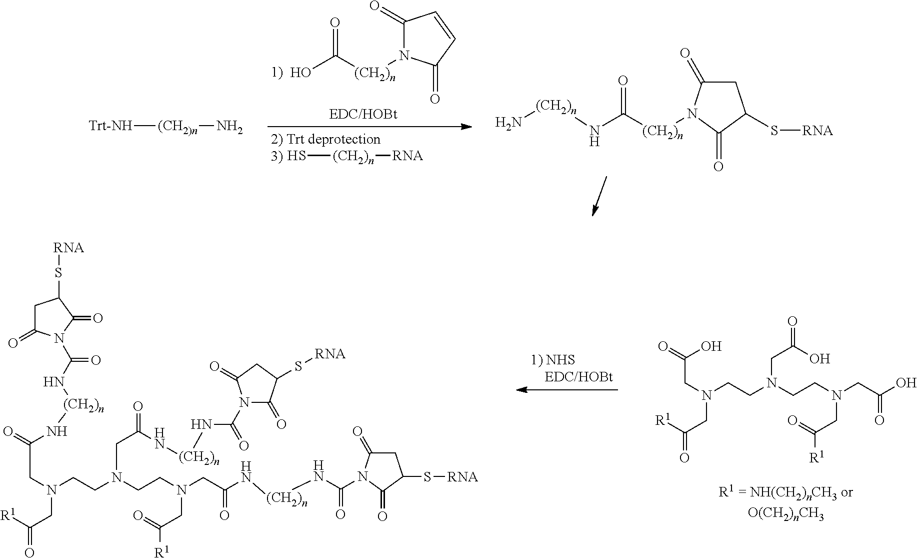

As shown in the schematic above, one eq of RNA molecule modified with an HS-(CH₂)ₙ-linker is dissolved in DMF/20 mM TRIS buffer pH 7, and 1.2 eq of a diamine linker (NH₂—(CH₂)ₙ—NH₂) coupled to a maleimido carboxylic acid (maleimido-(CH₂)ₙ—COOH) is added (n is from 1 to 22). In this embodiment, the alkyl chain is —(CH₂)ₙ—, preferably where n is from 1 to 6. The intermediate product is purified by RP-HPLC. A dialkylated multi-arm amine compound (synthesis described previously) is dissolved in CH₂Cl₂ and 4 eq of NHS, followed by 3 eq of DIPEA. After the reaction is complete, the organic layer is washed three times with 1M NaHCO₃ aqueous solution, water followed by brine. The CH₂Cl₂ is dried with Mg(SO₄)₂ and is removed under reduced pressure. The crude NHS ester is used in a reaction with 4 eq of NH₂—(CH₂)ₙ—NH—C(O)—(CH₂)ₙ-maleimido-S-RNA in DMF/20 mM TRIS buffer pH 7. The desired product may be purified by RP-HPLC.

Synthesis of a monoalkylated multi-arm amine compound with attached RNA molecule(s) may be as follows:

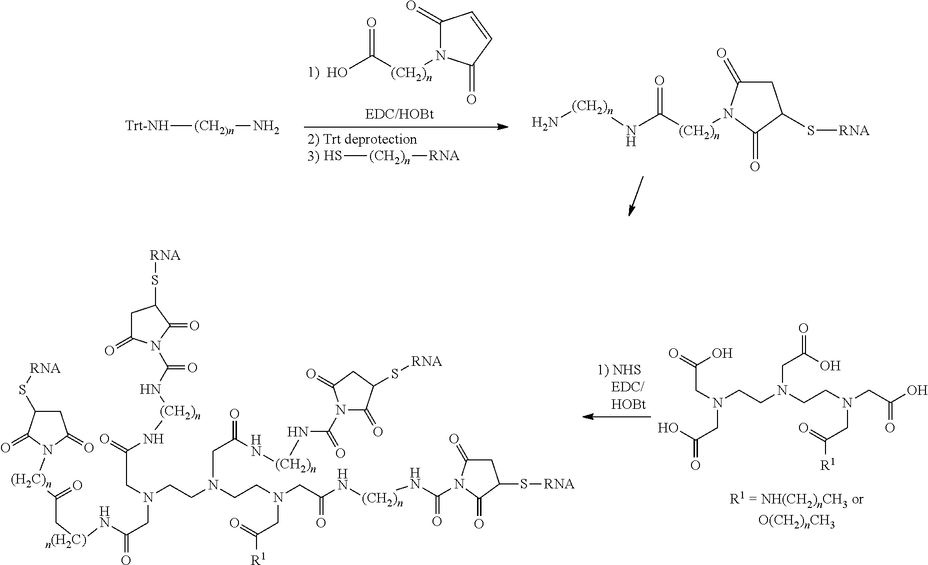

As shown in the schematic above, one eq of RNA molecule modified with an HS-(CH₂)ₙ-linker is dissolved in DMF/20 mM TRIS buffer pH 7, and 1.2 eq of a diamine linker (NH₂—(CH₂)ₙ—NH₂) coupled to a maleimido carboxylic acid (maleimido-(CH₂)ₙ—COOH) is added (n is from 1 to 22). In this embodiment, the alkyl chain is —(CH₂)ₙ—, preferably where n is from 1 to 6. The intermediate product is purified by RP-HPLC. A dialkylated multi-arm amine compound (synthesis described previously) is dissolved in CH₂Cl₂ and 5 eq of NHS, followed by 4 eq of DIPEA. After the reaction is complete, the organic layer is washed three times with 1M NaHCO₃ aqueous solution, water followed by brine. The CH₂Cl₂ is dried with Mg(SO₄)₂ and is removed under reduced pressure. The crude NHS ester is used in a reaction with 5 eq of NH₂—(CH₂)ₙ—NH—C(O)—(CH₂)ₙ-maleimido-S-RNA in DMF/20 mM TRIS buffer pH 7. The desired product may be purified by RP-HPLC.

Synthesis of a trialkylated multi-arm amine compound with attached RNA molecule(s) may be as follows:

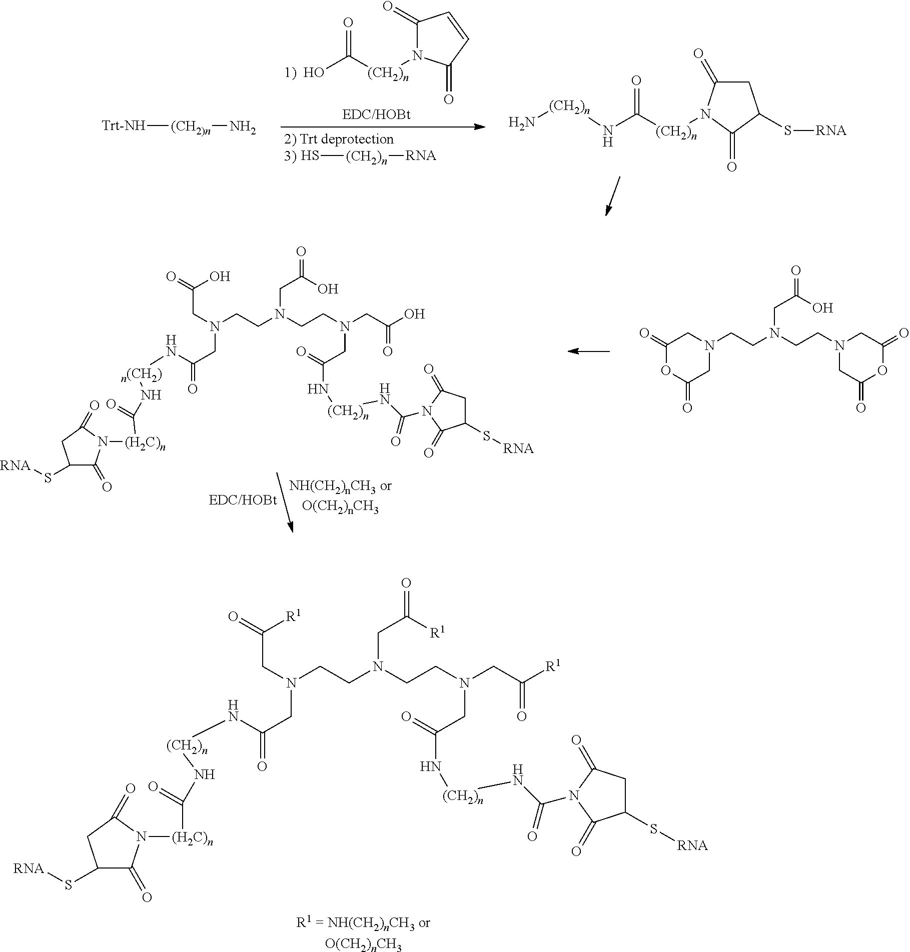

As shown in the schematic above, one eq of RNA molecule modified with an HS-(CH₂)ₙ-linker is dissolved in DMF/20 mM TRIS buffer pH 7, and 1.2 eq of a diamine linker (NH₂—(CH₂)ₙ—NH₂) coupled to a maleimido carboxylic acid (maleimido-(CH₂)ₙ—COOH) is added. The intermediate product is purified by RP-HPLC. 2-(bis(2-(2,6-dioxomorpholino)ethyl)amino)acetic acid is dissolved in DMF/20 mM TRIS buffer pH 7 and 3 eq NH₂—(CH₂)ₙ—NH—C(O)—(CH₂)ₙ-maleimido-S-RNA is added (n is from 1 to 22). In this embodiment, the alkyl chain is —(CH₂)ₙ—, preferably where n is from 1 to 6. After the reaction is complete, the desired compound is purified by RP-HPLC. The purified intermediate is dissolved in dry dimethylformamide and 4 eq of coupling regent (EDC×HCL/HOBt) is added, followed by 4 eq of CH₃(CH₂)ₙNH₂ amine and/or CH₃(CH₂)ₙOH (ester analogs; n dictates the length of the carbon tail attached). Protecting groups on the reactive moieties are added, if necessary. Lastly, 4 eq of DIPEA is added. After two hours, the reaction mixture is diluted with water and the desired product is extracted three times with CH₂Cl₂. The CH₂Cl₂ is dried with Mg(SO₄)₂ and is removed under reduced pressure. The desired product may be purified by RP-HPLC.

Synthesis of a tetraalkylated multi-arm amine compound with attached RNA molecule(s) may be as follows:

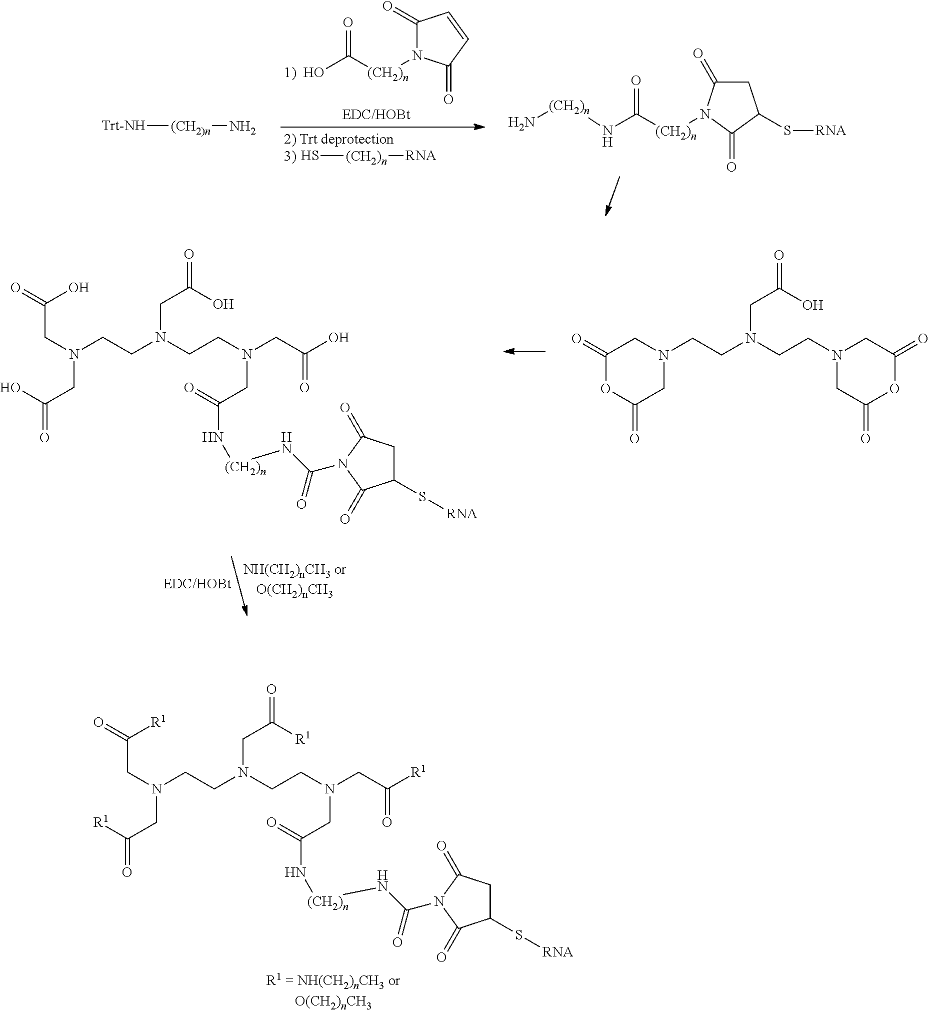

As shown in the schematic above, one eq of RNA molecule modified with an HS-(CH₂)ₙ-linker is dissolved in DMF/20 mM TRIS buffer pH 7, and 1.2 eq of a diamine linker (NH₂—(CH₂)ₙ—NH₂) coupled to a maleimido carboxylic acid (maleimido-(CH₂)ₙ—COOH) is added. The intermediate product is purified by RP-HPLC. 2-(bis(2-(2,6-dioxomorpholino)ethyl)amino)acetic acid is dissolved in DMF/20 mM TRIS buffer pH 7 and ⅓ eq NH₂—(CH₂)ₙ—NH—C(O)—(CH₂)ₙ-maleimido-S-RNA is added (n is from 1 to 22). In this embodiment, the alkyl chain is —(CH₂)ₙ—, preferably where n is from 1 to 6. After the reaction is complete, the desired compound is purified by RP-HPLC. The purified intermediate is dissolved in dry dimethylformamide and 5 eq of coupling regent (EDC×HCL/HOBt) is added, followed by 5 eq of CH₃(CH₂)ₙNH₂ amine and/or CH₃(CH₂)ₙOH (ester analogs; n dictates the length of the carbon tail attached). Protecting groups on the reactive moieties are added, if necessary. Lastly, 4 eq of DIPEA is added. After two hours, the reaction mixture is diluted with water and the desired product is extracted three times with CH₂Cl₂. The CH₂Cl₂ is dried with Mg(SO₄)₂ and is removed under reduced pressure. The desired product may be purified by RP-HPLC.

The following are representative hydroxyl protecting groups that may be used in the methods described herein, Examples of hydroxyl protecting groups include, but are not limited to, t-butyl, t-butoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, 2,6-dichlorobenzyl, diphenylmethyl, p,p′-dinitrobenzhydryl, p-nitrobenzyl, triphenylmethyl, trimethylsilyl, triethylsilyl, t-butyldimethylsilyl, t-butyldiphenylsilyl, triphenylsilyl, benzoylformate, acetate, chloroacetate, trichloroacetate, trifluoroacetate, pivaloate, benzoate, p-phenylbenzoate, 9-fluorenylmethyl carbonate, mesylate and tosylate. Further examples are disclosed by Beaucage et al. (Tetrahedron, 1992, 48:2223-2311). Further hydroxyl protecting groups, as well as other representative protecting groups, are disclosed in Greene and Wuts, Protective Groups in Organic Synthesis, Chapter 2, 2d ed., John Wiley & Sons, New York, 1991, and Oligonucleotides And Analogues A Practical Approach, Ekstein, F. Ed., IRL Press, N.Y., 1991.

Amino-protecting groups stable to acid treatment are selectively removed with base treatment, and are used to make reactive amino groups selectively available for substitution. Examples of such groups are the Fmoc (E. Atherton and R. C. Sheppard in The Peptides, S. Udenfriend, J. Meienhofer, Eds., Academic Press, Orlando, 1987, volume 9, p. 1) and various substituted sulfonylethyl carbamates exemplified by the Nsc group (Samukov et al., Tetrahedron Lett., 1994, 35:7821; Verhart and Tesser, Rec. Tray. Chim. Pays-Bas, 1987, 107:621).

Additional amino-protecting groups include, but are not limited to, carbamate protecting groups, such as 2-trimethylsilylethoxycarbonyl (Teoc), 1-methyl-1-(4-biphenylyl)ethoxycarbonyl (Bpoc), t-butoxycarbonyl (BOC), allyloxycarbonyl (Alloc), 9-fluorenylmethyloxycarbonyl (Fmoc), and benzyloxycarbonyl (Cbz); amide protecting groups, such as formyl, acetyl, trihaloacetyl, benzoyl, and nitrophenylacetyl; sulfonamide protecting groups, such as 2-nitrobenzenesulfonyl; and imine and cyclic imide protecting groups, such as phthalimido and dithiasuccinoyl.

Certain multi-arm amine compounds of this disclosure may exist in particular geometric or stereoisomeric forms. Unless specified otherwise, the present disclosure contemplates all such compounds, including cis- and trans-isomers, R- and S-enantiomers, diastereomers, (D)-isomers, (L)-isomers, the racemic mixtures thereof, and other mixtures thereof, as falling within the scope of the disclosure. Additional asymmetric carbon atoms may be present in a substituent such as an alkyl group. All such isomers, as well as mixtures thereof, are intended to be included in the multi-arm amine compounds of this disclosure.

### Uses for RNA Therapeutics

In some aspects, this disclosure relates to compounds and compositions for delivery of ribonucleic acids, and their uses for medicaments and for delivery as therapeutics. This disclosure relates generally to methods of using ribonucleic acids or regulatory RNA. One way to use a therapeutic ribonucleic acid is in RNA interference for gene-specific inhibition of gene expression in mammals.

RNA interference (RNAi) refers to methods of sequence-specific post-transcriptional gene silencing which is mediated by a double-stranded RNA (dsRNA) called a short interfering RNA (siRNA). See Fire, et al., Nature 391:806, 1998, and Hamilton, et al., Science 286:950-951, 1999. RNAi is shared by diverse flora and phyla and is believed to be an evolutionarily-conserved cellular defense mechanism against the expression of foreign genes. See Fire, et al., Trends Genet. 15:358, 1999.

RNAi is therefore a ubiquitous, endogenous mechanism that uses small noncoding RNAs to silence gene expression. See Dykxhoorn, D. M. and J. Lieberman, Annu. Rev. Biomed. Eng. 8:377-402, 2006. RNAi can regulate important genes involved in cell death, differentiation, and development. RNAi may also protect the genome from invading genetic elements, encoded by transposons and viruses. When a siRNA is introduced into a cell, it binds to the endogenous RNAi machinery to disrupt the expression of mRNA containing complementary sequences with high specificity. Any disease-causing gene and any cell type or tissue can potentially be targeted. This technique has been rapidly utilized for gene-function analysis and drug-target discovery and validation. Harnessing RNAi also holds great promise for therapy, although introducing siRNAs into cells in vivo remains an important obstacle.

The mechanism of RNAi, although not yet fully characterized, is through cleavage of a target mRNA. The RNAi response involves an endonuclease complex known as the RNA-induced silencing complex (RISC), which mediates cleavage of a single-stranded RNA complementary to the antisense strand of the siRNA duplex. Cleavage of the target RNA takes place in the middle of the region complementary to the antisense strand of the siRNA duplex. See, e.g., Elbashir, et al., Genes Dev. 15:188 (2001).

One way to carry out RNAi is to introduce or express a siRNA in cells. Another way is to make use of an endogenous ribonuclease III enzyme called dicer. One activity of dicer is to process a long dsRNA into siRNAs. See Hamilton, et al., Science 286:950-951, 1999; Berstein, et al., Nature 409:363, 2001. A siRNA derived from dicer is typically about 21-23 nucleotides in overall length with about 19 base pairs duplexed. See Hamilton, et al., supra; Elbashir, et al., Genes Dev. 15:188, 2001. In essence, a long dsRNA can be introduced in a cell as a precursor of a siRNA.

This disclosure provides a range of compositions, formulations and methods which include an interfering nucleic acid or a precursor thereof in combination with various components including novel multi-arm amine compounds, lipids, amino acid lipids, and natural or synthetic polymers.

The compositions and formulations of this disclosure may be used for delivery of RNAi-inducing entities such as dsRNA, siRNA, shRNA, miRNA, mdRNA, or RNAi-inducing vectors to cells in intact mammalian subjects, and may also be used for delivery of these agents to cells in culture.

This disclosure also provides methods for the delivery of one or more RNAi-inducing entities to organs and tissues within the body of a mammal. In some embodiments, compositions containing an RNAi-inducing entity and one or more multi-arm amine compounds, along with additional components, are introduced by various routes to be transported within the body and taken up by cells in one or more organs or tissues, where expression of a target transcript is modulated.

The term “nucleic acid” as used herein refers to a single-stranded or double-stranded RNA or DNA molecule, peptide nucleic acid (PNA), morpholino, glycol nucleic acid (GNA), threose nucleic acid (TNA), and combination thereof.

The term “dsRNA” as used herein refers to any nucleic acid molecule comprising at least one ribonucleotide molecule and capable of inhibiting or down regulating gene expression, for example, by promoting RNA interference (“RNAi”) or gene silencing in a sequence-specific manner. The dsRNAs of this disclosure may be suitable substrates for Dicer or for association with RISC to mediate gene silencing by RNAi. One or both strands of the dsRNA can further comprise a terminal phosphate group, such as a 5′-phosphate or 5′,3′-diphosphate. As used herein, dsRNA molecules, in addition to at least one ribonucleotide, can further include substitutions, chemically-modified nucleotides, and non-nucleotides. In certain embodiments, dsRNA molecules comprise ribonucleotides up to about 100% of the nucleotide positions.

As used herein, the term RNAi is synonymous with the term iRNA, both of which refer to RNA interference and interfering RNA.

In one aspect, a dsRNA comprises two separate oligonucleotides, comprising a first strand (antisense) and a second strand (sense), wherein the antisense and sense strands are self-complementary (i.e., each strand comprises a nucleotide sequence that is complementary to a nucleotide sequence in the other strand and the two separate strands form a duplex or double-stranded structure, for example, wherein the double-stranded region is about 10 to about 29 base pairs or about 10, 11, 12, 13, 14, 15, 16, 17, 18, 29, 20, 21, 22, 23, 24, 25, 26, 27, 28, or 29 base pairs, or about 29 to about 40 base pairs or 29, 30, 31, 32, 33, 34, 35, 36, 37, 38, 39, or 40 base pairs); the antisense strand comprises a nucleotide sequence that is complementary to a nucleotide sequence in a target nucleic acid molecule or a portion thereof; and the sense strand comprises a nucleotide sequence corresponding (i.e., is homologous) to the target nucleic acid sequence or a portion thereof (e.g., a sense strand of about 10 to about 29 nucleotides or about 10, 11, 12, 13, 14, 15, 16, 17, 18, 29, 20, 21, 22, 23, 24, 25, 26, 27, 28, or 29 nucleotides, or about 29 to about 40 nucleotides or 29, 30, 31, 32, 33, 34, 35, 36, 37, 38, 39, or 40 nucleotides corresponds to the target nucleic acid or a portion thereof).

In yet another embodiment, the dsRNA has blunt ends.

In yet another embodiment, the dsRNA has one 3′ overhang of 1 to 5 (or 1, 2, 3, 4, 5) nucleotides.

In yet another embodiment, the dsRNA has two 3′ overhangs, each 3′ overhang independently having 1 to 5 (or 1, 2, 3, 4, 5) nucleotides.

In yet another embodiment, the dsRNA has an overhang of more than five nucleotides.

The dsRNA may be complexed or associated (i.e., non-covalent interaction) with the multi-arm amine compound of the disclosure. One or more dsRNAs having the same or different nucleic acid sequence may be complexed or associated with the multi-arm amine compound of the disclosure.

The dsRNA may be covalently linked to multi-arm amine compound. One or more dsRNAs having the same or different nucleic acid sequence may be covalently linked to the multi-arm amine compound of the disclosure.

Further, the compositions, formulations and uses disclosed herein for delivery of nucleic acid agents are amenable to delivery of single stranded RNA agents including antisense oligomers.

Examples of dsRNA molecules can be found in, for example, U.S. patent application Ser. No. 11/681,725, U.S. Pat. Nos. 7,022,828 and 7,034,009, and PCT International Application Publication No. WO/2003/070897. Examples of human genes suitable as therapeutic targets and nucleic acid sequences thereto include those disclosed in PCT/US08/55333, PCT/US08/55339, PCT/US08/55340, PCT/US08/55341, PCT/US08/55350, PCT/US08/55353, PCT/US08/55356, PCT/US08/55357, PCT/US08/55360, PCT/US08/55362.

In addition, as used herein, the terms “dsRNA,” “RNAi-inducing agent,” “RNAi-agent,” and “interfering RNA agent” are meant to be synonymous with other terms used to describe nucleic acid molecules that are capable of mediating sequence specific RNAi including meroduplex RNA (mdRNA), nicked dsRNA (ndsRNA), gapped dsRNA (gdsRNA), short interfering nucleic acid (siNA), siRNA, microRNA (miRNA), short hairpin RNA (shRNA), short interfering oligonucleotide, short interfering substituted oligonucleotide, short interfering modified oligonucleotide, chemically-modified dsRNA, and post-transcriptional gene silencing RNA (ptgsRNA), among others.

An mdRNA molecule is an RNA molecule having at least three strands and at least two duplex regions separated by a “gap”, i.e., it contains a “gap” ranging from 1 nucleotides up to about 10 nucleotides (or a gap of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 nucleotides) or a “nick”, i.e., the phosphodiester bond between two adjacent nucleotides of the same strand is absent, whereby one strand of the RNA molecule is a continuous strand (i.e., the “A” strand) and the other strand is discontinuous or is made of at least two separate strands (i.e., the “S1” and “S2” strand). The duplex regions result from the 51 and S2 strands annealing to the A strand. In one embodiment, the two duplex regions are separated by a gap resulting from at least one unpaired nucleotide (up to about 10 unpaired nucleotides) in the ‘A’ strand that is positioned between the A:S1 duplex and the A: S2 duplex and that is distinct from any one or more unpaired nucleotide at the 3′-end of one or more of the ‘A’, ‘S1’, or ‘S2’ strands. In another embodiment, the A:S1 duplex is separated from the A:S2 duplex by a nick (i.e., a nick in which only a phosphodiester bond between two nucleotides is broken or missing in the polynucleotide molecule) between the A:S1 duplex and the A: S2 duplex—which can also be referred to as nicked dsRNA (ndsRNA). For example, A:S1S2 may be comprised of a dsRNA having at least two double-stranded regions that combined total about 14 base pairs to about 40 base pairs and the double-stranded regions are separated by a gap of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 nucleotides, optionally having blunt ends, or A:S1S2 may comprise a dsRNA having at least two double-stranded regions spaced apart by up to 10 nucleotides and thereby forming a gap between the second and third strands wherein at least one of the double-stranded regions optionally has from 5 base pairs to 13 base pairs.

### Compositions and Formulations for Administration

The compositions and formulations of this disclosure may be administered by various routes, for example, to effect systemic delivery via intravenous, parenteral, or intraperitoneal routes. In some embodiments, an active agent may be delivered intracellularly, for example, in cells of a target tissue such as lung or liver, or in inflamed tissues. Included within this disclosure are compositions and methods for delivery of an active agent by removing cells of a subject, delivering the agent to the removed cells, and reintroducing the cells into a subject. In some embodiments, this disclosure provides a method for delivery of an active agent in vivo. A composition of this disclosure may be administered intravenously, subcutaneously, or intraperitoneally to a subject. In some embodiments, this disclosure provides methods for in vivo delivery of an active agent to the lung of a mammalian subject. In some embodiments, the disclosure provides a method of administering whereby the multi-arm amine and the drug, biological agent, or API are administered at the same time. In other embodiments, the disclosure provides a method of administering whereby the multi-arm amine is administered prior to the drug, biological agent, or API. In such embodiments, the multi-arm amine is administered from 30 seconds to 8 hours before the drug, biological agent, or API is administered, preferably 1 minute to 4 hours before, more preferably 5 minutes to 2 hours before, and most preferably 15 minutes to 2 hours before.

The excipients and other formulation components disclosed herein or incorporated by reference herein enhance and/or improve chemically stability or physical stability of the compositions and formulations disclosed herein.

In some embodiments, this disclosure provides a method of treating a disease or disorder in a mammalian subject. A therapeutically effective amount of a composition of this disclosure may be administered to a subject having a disease or disorder associated with expression or overexpression of a gene that can be reduced, decreased, downregulated, or silenced by the composition.

This disclosure encompasses methods for treating a disease of the lung such as respiratory distress, asthma, cystic fibrosis, pulmonary fibrosis, chronic obstructive pulmonary disease, bronchitis, or emphysema, by administering to the subject a therapeutically effective amount of a composition.

This disclosure encompasses methods for treating rheumatoid arthritis, liver disease, encephalitis, bone fracture, heart disease, viral disease including hepatitis and influenza, or cancer, such as liver cancer or bladder cancer.

This disclosure encompasses methods for treating inflammation, osteoarthritis, atherosclerosis, dermatitis, bone resorption diseases, reperfusion injury, asthma, multiple sclerosis, Guillain-Barre syndrome, Crohn's disease, ulcerative colitis, psoriasis, systemic lupus erythematosus, insulin-dependent diabetes mellitus, rheumatoid arthritis, toxic shock syndrome, Alzheimer's disease, autism, diabetes, inflammatory bowel diseases, acute and chronic pain, stroke, myocardial infarction, meningitis, entrerocolitis, restenosis, sepsis, and chronic obstructive pulmonary disease.

The compositions and methods of this disclosure may be administered to subjects by a variety of mucosal administration modes, including by oral, rectal, vaginal, intranasal, intrapulmonary, or transdermal delivery, or by topical delivery to the eyes, ears, skin or other mucosal surfaces. In some aspects of this disclosure, the mucosal tissue layer includes an epithelial cell layer. The epithelial cell can be pulmonary, tracheal, bronchial, alveolar, nasal, buccal, epidermal, or gastrointestinal. Compositions of this disclosure can be administered using conventional actuators such as mechanical spray devices, as well as pressurized, electrically activated, or other types of actuators.

Compositions of this disclosure may be administered in an aqueous solution as a nasal or pulmonary spray and may be dispensed in spray form by a variety of methods known to those skilled in the art. Pulmonary delivery of a composition of this disclosure may be achieved by administering the composition in the form of drops, particles, or spray, which can be, for example, aerosolized, atomized, or nebulized. Pulmonary delivery may be performed by administering the composition in the form of drops, particles, or spray, via the nasal or bronchial passages. Particles of the composition, spray, or aerosol can be in a either liquid or solid form. Preferred systems for dispensing liquids as a nasal spray are disclosed in U.S. Pat. No. 4,511,069. Such formulations may be conveniently prepared by dissolving compositions according to the present disclosure in water to produce an aqueous solution, and rendering said solution sterile. The formulations may be presented in multi-dose containers, for example in the sealed dispensing system disclosed in U.S. Pat. No. 4,511,069. Other suitable nasal spray delivery systems have been described in Transdermal Systemic Medication, Y. W. Chien ed., Elsevier Publishers, New York, 1985; and in U.S. Pat. No. 4,778,810. Additional aerosol delivery forms may include, for example, compressed air-, jet-, ultrasonic-, and piezoelectric nebulizers, which deliver the biologically active agent dissolved or suspended in a pharmaceutical solvent, for example, water, ethanol, or mixtures thereof.

Nasal and pulmonary spray solutions of the present disclosure typically comprise the drug or drug to be delivered, optionally formulated with a surface active agent, such as a nonionic surfactant (e.g., polysorbate-80, polysorbate-20, polysorbate-60, or L-α-phosphatidylcholine didecanoyl), and one or more buffers. In some embodiments of the present disclosure, the nasal spray solution further comprises a propellant. The pH of the nasal spray solution may be from about pH 3 to 11, or from 6.8 to 7.2. The pharmaceutical solvents employed can also be a slightly acidic aqueous buffer of pH 4-6. Other components may be added to enhance or maintain chemical stability or physical stability, including preservatives, surfactants, dispersants, or gases.

In some embodiments, this disclosure is a pharmaceutical product which includes a solution containing a composition of this disclosure and an actuator for a pulmonary, mucosal, or intranasal spray or aerosol.

A dosage form of the composition of this disclosure can be liquid, in the form of droplets or an emulsion, or in the form of an aerosol.

A dosage form of the composition of this disclosure can be solid, which can be reconstituted in a liquid prior to administration. The solid can be administered as a powder. The solid can be in the form of a capsule, tablet or gel.

To formulate compositions for pulmonary delivery within the present disclosure, the biologically active agent can be combined with various pharmaceutically acceptable additives or delivery-enhancing components, as well as a base or carrier for dispersion of the active agent(s). Examples of additives or delivery-enhancing components include pH control agents such as arginine, sodium hydroxide, glycine, hydrochloric acid, citric acid, and mixtures thereof. Other additives or delivery-enhancing components include local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione). When the composition for mucosal delivery is a liquid, the tonicity of the formulation, as measured with reference to the tonicity of 0.9% (w/v) physiological saline solution taken as unity, is typically adjusted to a value at which no substantial, irreversible tissue damage will be induced in the mucosa at the site of administration. Generally, the tonicity of the solution is adjusted to a value of about ⅓ to 3, more typically ½ to 2, and most often ¾ to 1.7.

The biologically active agent may be dispersed in a base or vehicle, which may comprise a hydrophilic compound having a capacity to disperse the active agent and any desired additives. The base may be selected from a wide range of suitable carriers, including but not limited to, copolymers of polycarboxylic acids or salts thereof, carboxylic anhydrides (e.g., maleic anhydride) with other monomers (e.g., methyl (meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. A biodegradable polymer may be selected as a base or carrier, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters, etc., can be employed as carriers. Hydrophilic polymers and other carriers can be used alone or in combination, and enhanced structural integrity can be imparted to the carrier by partial crystallization, ionic bonding, crosslinking and the like. The carrier can be provided in a variety of forms, including, fluid or viscous solutions, gels, pastes, powders, microspheres and films for direct application to the nasal mucosa. The use of a selected carrier in this context may result in promotion of absorption of the biologically active agent.

The biologically active agent can be combined with the base or carrier according to a variety of methods, and release of the active agent may be by diffusion, disintegration of the carrier, or associated formulation of water channels. In some circumstances, the active agent is dispersed in microcapsules (microspheres) or nanocapsules (nanospheres) prepared from a suitable polymer, e.g., isobutyl 2-cyanoacrylate (see, e.g., Michael, et al., J. Pharmacy Pharmacol. 43:1-5, 1991), and dispersed in a biocompatible dispersing medium applied to the nasal mucosa, which yields sustained delivery and biological activity over a protracted time.

Formulations for mucosal, nasal, or pulmonary delivery may contain a hydrophilic low molecular weight compound as a base or excipient. Such hydrophilic low molecular weight compounds provide a passage medium through which a water-soluble active agent, such as a physiologically active peptide or protein, may diffuse through the base to the body surface where the active agent is absorbed. The hydrophilic low molecular weight compound optionally absorbs moisture from the mucosa or the administration atmosphere and dissolves the water-soluble active peptide. The molecular weight of the hydrophilic low molecular weight compound is generally not more than 10,000 and preferably not more than 3000. Examples of hydrophilic low molecular weight compounds include polyol compounds, such as oligo-, di- and monosaccarides including sucrose, mannitol, lactose, L-arabinose, D-erythrose, D-ribose, D-xylose, D-mannose, D-galactose, lactulose, cellobiose, gentibiose, glycerin, polyethylene glycol, and mixtures thereof. Further examples of hydrophilic low molecular weight compounds include N-methylpyrrolidone, alcohols (e.g., oligovinyl alcohol, ethanol, ethylene glycol, propylene glycol, etc.), and mixtures thereof.

The compositions of this disclosure may alternatively contain as pharmaceutically acceptable carriers substances as required to approximate physiological conditions, such as pH adjusting and buffering agents, tonicity adjusting agents, and wetting agents, for example, sodium acetate, sodium lactate, sodium chloride, potassium chloride, calcium chloride, magnesium chloride, sorbitan monolaurate, triethanolamine oleate, and mixtures thereof. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

In certain embodiments of the disclosure, the biologically active agent may be administered in a time release formulation, for example in a composition which includes a slow release polymer. The active agent can be prepared with carriers that will protect against rapid release, for example a controlled release vehicle such as a polymer, microencapsulated delivery system or bioadhesive gel. Prolonged delivery of the active agent, in various compositions of the disclosure can be brought about by including in the composition agents that delay absorption, for example, aluminum monosterate hydrogels and gelatin.

Within certain embodiments of this disclosure, a composition may contain one or more natural or synthetic surfactants. Certain natural surfactants are found in human lung (pulmonary surfactant), and are a complex mixture of phospholipids and proteins that form a monolayer at the alveolar air-liquid interface and reduces surface tension to near zero at expiration and prevents alveolar collapse. Over 90% (by weight) of pulmonary surfactant is composed of phospholipids with approximately 40-80% being DPPC and the remainder being unsaturated phosphatidylcholines POPG, POPC and phosphatidylglycerols. The remaining 10% (by weight) of surfactant is composed of plasma proteins and apoproteins, such as surface proteins (SP)-A, SP-B, SP-C and SP-D.

Examples of natural surfactants that may be used in this disclosure include SURVANTA (beractant), CUROSURF (poractant alfa) and INFASURF (calfactant), and mixtures thereof.

Examples of synthetic surfactants include sinapultide; a combination of dipalmitoylphosphatidylcholine, palmitoyloleoyl phosphatidylglycerol and palmitic acid; SURFAXIN (lucinactant); and EXOSURF (colfosceril); components which may contain tyloxapol, DPPC, and hexadecanol; and mixtures thereof.

One or more viscosity enhancing, thickening or suspending agents may be used within the context of this disclosure in order to affect the rate of release of a drug from the dosage formulation and absorption. Some examples of the materials which can serve as pharmaceutically acceptable viscosity enhancing agents are methylcellulose (MC); hydroxypropylmethylcellulose (HPMC); hydroxypropylmethylcellulose acetate succinate (HPMCAS), carboxymethylcellulose (CMC); cellulose; gelatin; starch; heta starch; poloxamers; pluronics; sodium CMC; sorbitol; acacia; povidone; carbopol; polycarbophil; chitosan; chitosan microspheres; alginate microspheres; chitosan glutamate; amberlite resin; hyaluronan; ethyl cellulose; maltodextrin DE; drum-dried way maize starch (DDWM); degradable starch microspheres (DSM); deoxyglycocholate (GDC); hydroxyethyl cellulose (HEC); hydroxypropyl cellulose (HPC); microcrystalline cellulose (MCC); polymethacrylic acid and polyethylene glycol; sulfobutylether B cyclodextrin; cross-linked eldexomer starch biospheres; sodiumtaurodihydrofusidate (STDHF); N-trimethyl chitosan chloride (TMC); degraded starch microspheres; amberlite resin; chistosan nanoparticles; spray-dried crospovidone; spray-dried dextran microspheres; spray-dried microcrystalline cellulose; and cross-linked eldexomer starch microspheres. Additional viscosity enhancing, thickening or suspending agents may include solutions or suspensions in the form of an in situ gel forming formulation, containing, for example, a PEG-containing formulation such as CoSeal®, SprayGel® and DuraSeal®; as well as collagen- and/or thrombin-containing formulations such as CoStasis®, and fibrin-containing formulations such as Tisseel®, and gelatin-containing formulations such as FloSeal®.

Compositions are provided that incorporate one or more selected from sodium salicylate and salicylic acid derivatives (acetyl salicylate, choline salicylate, salicylamide, etc.); amino acids and salts thereof (e.g. monoaminocarboxlic acids such as glycine, alanine, phenylalanine, proline, hydroxyproline, etc.; hydroxyamino acids such as serine; acidic amino acids such as aspartic acid, glutamic acid, etc; and basic amino acids such as lysine etc—inclusive of their alkali metal or alkaline earth metal salts); and N-acetylamino acids (N-acetylalanine, N-acetylphenylalanine, N-acetylserine, N-acetylglycine, N-acetyllysine, N-acetylglutamic acid, N-acetylproline, N-acetylhydroxyproline, etc.) and their salts (alkali metal salts and alkaline earth metal salts). Also provided as penetration-promoting agents within the methods and compositions of the disclosure are substances which are generally used as emulsifiers (e.g. sodium oleyl phosphate, sodium lauryl phosphate, sodium lauryl sulfate, sodium myristyl sulfate, polyoxyethylene alkyl ethers, polyoxyethylene alkyl esters, etc.), caproic acid, lactic acid, malic acid and citric acid and alkali metal salts thereof, pyrrolidonecarboxylic acids, alkylpyrrolidonecarboxylic acid esters, N-alkylpyrrolidones, proline acyl esters, and the like.

Compositions of this disclosure can be prepared by methods known in the art. Methods of making lipid compositions include ethanol injection methods and extrusion methods using a Northern Lipids Lipex Extruder system with stacked polycarbonate membrane filters of defined pore size. Sonication using probe tip and bath sonicators can be employed to produce lipid particles of uniform size. Homogenous and monodisperse particle sizes can be obtained without the addition of the nucleic acid component. For in vitro transfection compositions, the nucleic acid component can be added after the transfection agent is made and stabilized by additional buffer components. For in vivo delivery compositions, the nucleic acid component is part of the formulation.

Supplemental or complementary methods for delivery of nucleic acid molecules for use within then disclosure are described, for example, in Akhtar et al., Trends Cell Bio. 2:139, 1992; “Delivery Strategies for Antisense Oligonucleotide Therapeutics,” ed. Akhtar, 1995, Maurer et al., Mol. Membr. Biol. 16:129-140, 1999; Hofland and Huang, Handb. Exp. Pharmacol. 137:165-192, 1999; and Lee et al., ACS Symp. Ser. 752:184-192, 2000. Sullivan, et al., International PCT Publication No. WO 94/02595, further describes general methods for delivery of enzymatic nucleic acid molecules.

Nucleic acid molecules can be administered within formulations that include one or more multi-arm amine compounds of this disclosure, as well as additional components such as a pharmaceutically acceptable carrier, diluent, excipient, adjuvant, emulsifier, buffer, stabilizer, or preservative.

As used herein, the term “carrier” means a pharmaceutically acceptable solid or liquid filler, solvent, diluent or encapsulating material. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents, protamines (e.g., protamine sulfate) or other biocompatible materials. Examples of ingredients of the above categories can be found in the U.S. Pharmacopeia National Formulary, 1990, pp. 1857-1859, as well as in Raymond C. Rowe, et al., Handbook of Pharmaceutical Excipients, 5th ed., 2006, and “Remington: The Science and Practice of Pharmacy,” 21st ed., 2006, editor David B. Troy.

Examples of preservatives include phenol, methyl paraben, paraben, m-cresol, thiomersal, benzylalkonium chloride, and mixtures thereof.

Examples of surfactants include oleic acid, sorbitan trioleate, polysorbates, lecithin, phosphotidylcholines, various long chain diglycerides and phospholipids, and mixtures thereof.

Examples of phospholipids include phosphatidylcholine, lecithin, phosphatidylglycerol, phosphatidylinositol, phosphatidylserine, and phosphatidylethanolamine, and mixtures thereof.

In certain embodiments, the active agent can be encapsulated in liposomes, or reside either internal or external to a liposome, or exist within liposome layers, or be administered by iontophoresis, or incorporated into other vehicles, such as hydrogels, cyclodextrins (e.g., γ-cyclodextrin, α-cyclodextrin, methyl-β-cyclodextrin, 2-hydroxypropyl-β-cyclodextrin and heptakis(2,6-di-O-methyl-β-cyclodextrin)), biodegradable nanocapsules, bioadhesive microspheres, or proteinaceous vectors. See, for example, O'Hare and Normand, PCT International Publication No. WO 00/53722. Alternatively, a composition can be locally delivered by direct injection or by use of an infusion pump. Direct injection of the nucleic acid molecules of the disclosure, whether subcutaneous, intramuscular, or intradermal, can take place using standard needle and syringe methodologies, or by needle-free technologies such as those described in Conry et al., Clin. Cancer Res. 5:2330-2337, 1999, and Barry et al., International PCT Publication No. WO 99/31262.

In certain embodiments, the multi-arm amine is used in a method or composition of this disclosure in a range of from about 0.001 nM to about 1 M. In some embodiments, the multi-arm amine is used in a method or composition of this disclosure in a range of from about 0.001 mg/mL to 500 mg/mL.

### Additional Embodiments

All publications, references, patents, patent publications and patent applications cited herein are each hereby specifically incorporated by reference in its entirety.

While this disclosure has been described in relation to certain embodiments, and many details have been set forth for purposes of illustration, it will be apparent to those skilled in the art that this disclosure includes additional embodiments, and that some of the details described herein may be varied considerably without departing from this disclosure. This disclosure includes such additional embodiments, modifications and equivalents. In particular, this disclosure includes any combination of the features, terms, or elements of the various illustrative components and examples.

The use herein of the terms “a,” “an,” “the” and similar terms in describing the disclosure, and in the claims, are to be construed to include both the singular and the plural.

The terms “comprising,” “having,” “including” and “containing” are to be construed as open-ended terms which mean, for example, “including, but not limited to.” Thus, terms such as “comprising,” “having,” “including” and “containing” are to be construed as being inclusive, not exclusive.

Recitation of a range of values herein refers individually to each and any separate value falling within the range as if it were individually recited herein, whether or not some of the values within the range are expressly recited. For example, the range “4 to 12” includes without limitation the values 5, 5.1, 5.35 and any other whole or fractional value greater than or equal to 4 and less than or equal to 12. Specific values employed herein will be understood as exemplary and not to limit the scope of the disclosure.

Definitions of technical and scientific terms provided herein should be construed to include without recitation those meanings associated with the terms known to those skilled in the art, and are not intended to limit the scope of the disclosure. Definitions of technical terms expressly provided herein shall be construed to dominate over alternative definitions in the art or definitions which become incorporated herein by reference to the extent that the alternative definitions conflict with the express definition provided herein.

The examples given herein, and the exemplary language used herein are solely for the purpose of illustration, and are not intended to limit the scope of the disclosure.

When a list of examples is given, such as a list of compounds or molecules suitable for this disclosure, it will be apparent to those skilled in the art that mixtures of the listed compounds or molecules are also suitable.

### EXAMPLES

### Example 1

The following examples were prepared by reacting an amine with diethylenetriaminepentaacetic acid dianhydride and isolating the product by chromatograhpy.

To 20 g (55.97 mM) of diethylenetriaminepentaacetic acid dianhydride (Sigma; D6148-10g (042K006); CAS23911-26-4; Mw=357.32) suspended in 200 ml of NMP/200 ml of DCM/200 ml of DMF, 39 ml (223.88 mmol 4 eq) of DIEA was added followed by 28 ml (139.925 mM; 2.5 eq, Mw=157.3, d=0.787) of CH₃(CH₂)₉NH₂. Residue dissolved within 0.5 hr. After overnight reaction DCM was evaporated and the product was precipitated with 0.1 M HCl. The crude precipitate was suspended in H₂O and pH was adjusted to 8, the residue dissolved completely. The main product is dialkylamide, and the yield of monoalkylamide is less then 5%.

MA-(C10)monoalkylamide C₂₄H₄₄N₄O₉ was isolated using a ISCO COMBIFLASH chromatography separation instrument with a 330 g C18 silica gel column, 0% MeOH for 5 CV (column volume) (H2O mobile phase A), and 0-30% MeOH for 10 CV, 100% MeOH for 4 CV, detection 215 nm, flow 20 ml/min.

MA-(C10)monoalkylamide; C₂₄H₄₄N₄O₉; 2-[2-(bis(carboxymethyl)amino)ethyl-[2-[carboxymethyl-[2-(decylamino)-2-oxoethyl]amino]ethyl]amino]acetic acid

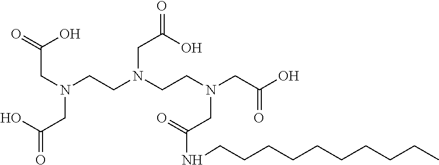

MA-(C12)monoalkylamide; C₂₆H₄₈N₄O₉; 2-[2-(bis(carboxymethyl)amino)ethyl-[2-[carboxymethyl-[2-(dodecylamino)-2-oxoethyl]amino]ethyl]amino]acetic acid

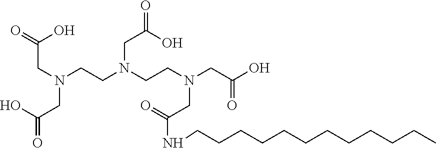

### Example 2

In like fashion as in Example 1 are made the following compounds:
MA-(C11)monoalkylamide; 2-[2-(bis(carboxymethyl)amino)ethyl-[2-[carboxymethyl-[2-(undecylamino)-2-oxoethyl]amino]ethyl]amino]acetic acid
MA-(C13)monoalkylamide; 2-[2-(bis(carboxymethyl)amino)ethyl-[2-[carboxymethyl-[2-(tridecylamino)-2-oxoethyl]amino]ethyl]amino]acetic acid
MA-(C14)monoalkylamide; 2-[2-(bis(carboxymethyl)amino)ethyl-[2-[carboxymethyl-[2-(tetradecylamino)-2-oxoethyl]amino]ethyl]amino]acetic acid
MA-(C15)monoalkylamide; 2-[2-(bis(carboxymethyl)amino)ethyl-[2-[carboxymethyl-[2-(pentadecylamino)-2-oxoethyl]amino]ethyl]amino]acetic acid
MA-(C16)monoalkylamide; 2-[2-(bis(carboxymethyl)amino)ethyl-[2-[carboxymethyl-[2-(hexadecylamino)-2-oxoethyl]amino]ethyl]amino]acetic acid
MA-(C17)monoalkylamide; 2-[2-(bis(carboxymethyl)amino)ethyl-[2-[carboxymethyl-[2-(heptadecylamino)-2-oxoethyl]amino]ethyl]amino]acetic acid
MA-(C18)monoalkylamide; 2-[2-(bis(carboxymethyl)amino)ethyl-[2-[carboxymethyl-[2-(octadecylamino)-2-oxoethyl]amino]ethyl]amino]acetic acid.

### Example 3

The following examples were prepared by reacting diethylenetriaminepentaacetic acid dianhydride with amines.

To 2 g (5.597 mM) of diethylenetriaminepentaacetic acid dianhydride (Sigma; D6148-10 g (042K006); CAS23911-26-4; Mw=357.32) suspended in 100 ml of DMSO, 3.12 ml (4 eq) of TEA was added followed by 8.4 ml (16.791 mM; 3 eq, Mw=31.02)) of 2M CH₃NH₂ in THF. Solution was turbid so additional 100 ml of DMSO was added. Residue dissolved within 1 hr. After overnight reaction, the solvent was evaporated partially and then product was lyophilized.

MA-(C1/C1)dialkylamide; C₁₆H₂₉N₅O₈, 5,8-bis(carboxymethyl)-2-(2-(methylamino)-2-oxoethyl)-10-oxo-2,5,8,11-tetraazadodecane-1-carboxylic acid

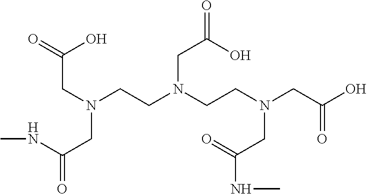

To 1.5 g (4.198 mM) of diethylenetriaminepentaacetic acid dianhydride (Sigma; D6148-10g (042K006); CAS23911-26-4; Mw=357.32) suspended in 50 ml of DCM, 2.922 ml (4 eq) of DIPEA was added followed by 2.221 ml (16.792 mM; 4 eq, Mw=101.19, d=0.765) of CH₃(CH₂)₅NH₂. Solution was turbid so additional 50 ml of DCM was added. Residue dissolved within 1 hr. After 3 hrs DCM was evaporated and the product was precipitated with H2O/AcCN solvent mixture, and then purified.

MA-(C6/C6)dialkylamide; 5,8-bis(carboxymethyl)-2-(2-(hexylamino)-2-oxoethyl)-10-oxo-2,5,8,11-tetraazaheptadecane-1-carboxylic acid

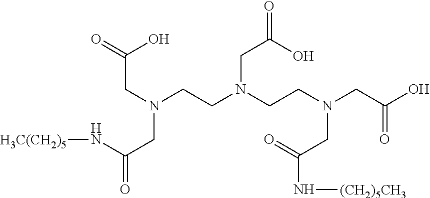

To 1.5 g (4.198 mM) of diethylenetriaminepentaacetic acid dianhydride (Sigma; D6148-10g (042K006); CAS23911-26-4; Mw=357.32) suspended in 50 ml of DCM, 2.922 ml (4 eq) of DIPEA was added followed by 2.775 ml (16.792 mM; 4 eq, Mw=129.25, d=0.782) of CH₃(CH₂)₇NH₂. Solution was turbid so additional 50 ml of DMSO was added. Residue dissolved within 3 hr. After overnight reaction DCM was evaporated and the product was precipitated with H2O/AcCN solvent mixture, and then purified. Yield: 1.09 g.

MA-(C8/C8)dialkylamide; 5,8-bis(carboxymethyl)-2-(2-(octylamino)-2-oxoethyl)-10-oxo-2,5,8,11-tetraazanonadecane-1-carboxylic acid

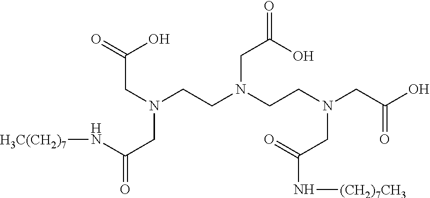

To 20 g (55.97 mM) of diethylenetriaminepentaacetic acid dianhydride (Sigma; D6148-10g (042K006); CAS23911-26-4; Mw=357.32) suspended in 200 ml of NMP/200 ml of DCM/200 ml of DMF, 39 ml (223.88 mmol 4 eq) of DIEA was added followed by 28 ml (139.925 mM; 2.5 eq, Mw=157.3, d=0.787) of CH₃(CH₂)₉NH₂. Residue dissolved within 0.5 hr. After overnight reaction DCM was evaporated and the product was precipitated with 0.1 M HCl. The crude precipitate was suspended in H2O and pH was adjusted to 8, the residue dissolved completely.

MA-(C10/C10)dialkylamide was isolated using a ISCO COMBIFLASH chromatography separation instrument with a 320 g C18 silica gel column, 30% MeOH for 5 CV(column volume) (H2O is mobile phase A) and 30-100% MeOH for 2 CV, 100% MeOH for 3 CV detection 215 nm, flow 55 ml/min.

MA-(C10/C10)dialkylamide; 5,8-bis(carboxymethyl)-2-(2-(decylamino)-2-oxoethyl)-10-oxo-2,5,8,11-tetraazahenicosane-1-carboxylic acid

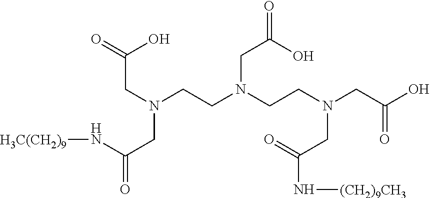

To 1.5 g (4.198 mM) of diethylenetriaminepentaacetic acid dianhydride (Sigma; D6148-10g (042K006); CAS23911-26-4; Mw=357.32) suspended in 150 ml of DCM, 2.922 ml (4 eq) of DIPEA was added followed by 3.112 g (16.792 mM; 4 eq, Mw=185.36) of CH₃(CH₂)₁₁NH₂. Solution was turbid so additional 100 ml of DMSO was added. Residue dissolved overnight. After overnight reaction DCM was evaporated and the product was precipitated with H2O/AcCN solvent mixture.

MA-(C12/C₁₋₂)dialkylamide; 5,8-bis(carboxymethyl)-2-(2-(dodecylamino)-2-oxoethyl)-10-oxo-2,5,8,11-tetraazatricosane-1-carboxylic acid

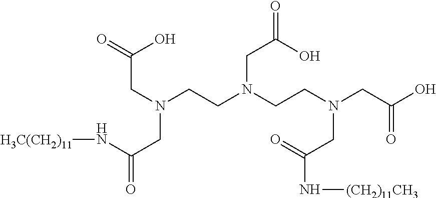

To 2 g (5.597 mM) of diethylenetriaminepentaacetic acid dianhydride (Sigma; D6148-10 g (042K006); CAS23911-26-4; Mw=357.32) suspended in 100 ml of chloroform, 3.12 ml (4 eq) of TEA was added followed by 4.05 9 (16.791 mM; 3 eq, Mw=241.4) of CH₃(CH₂)₁₅NH₂. Solution was turbid so additional 200 ml of CHCl₃ was added. Residue dissolved within 30 min. After overnight reaction, the solvent was evaporated and product crystallized from MeOH and ethyl ether. 1 g was converted to the Na+ form. Yield: 4 g.

MA-(C16/C16)dialkylamide; 5,8-bis(carboxymethyl)-2-(2-(hexadecylamino)-2-oxoethyl)-10-oxo-2,5,8,11-tetraazaheptacosane-1-carboxylic acid

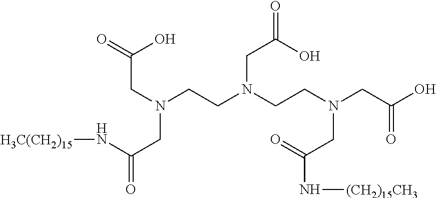

To 3 g (8.396 mM) of diethylenetriaminepentaacetic acid dianhydride (Sigma; D6148-10 g (042K006); CAS23911-26-4; Mw=357.32) suspended in 100 ml of DCM, 3.727 ml (4 eq) of DIPEA was added followed by 1.05 ml (2.09 mM; ¼ eq, Mw=31.06 of methylamine 2M solution and 0.417 ml (2.09 mM, ¼ eq, Mw=157.3, d=0.792) of decylamine Solution was turbid so additional 50 ml of DMSO was added. Residue dissolved within 2 hrs. After overnight reaction DCM was evaporated and a didecylamide byproduct was precipitated with a mixture of H2O/AcCN. The filtrate was loaded on C18 Phenomenex column (Phenomenex RP, 250×21.2 mm, Serial No.: 234236-1, Column volume 83 ml) and purified with 20-100% acetonitrile gradient using water as mobile phase within 2 CV; 215 nm. Acetonitrile was evaporated and the product was lyophilized. Converted to Na⁺ form with sodium bicarbonate. Yield: 0.223 g.

MA-(C1/C10)dialkylamide; C₂₅H₄₇N₅O₈; 5,8-bis(carboxymethyl)-2-(2-(methylamino)-2-oxoethyl)-10-oxo-2,5,8,11-tetraazahenicosane-1-carboxylic acid

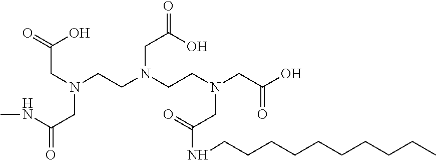

In like fashion as in Example 3 are made the following compounds:

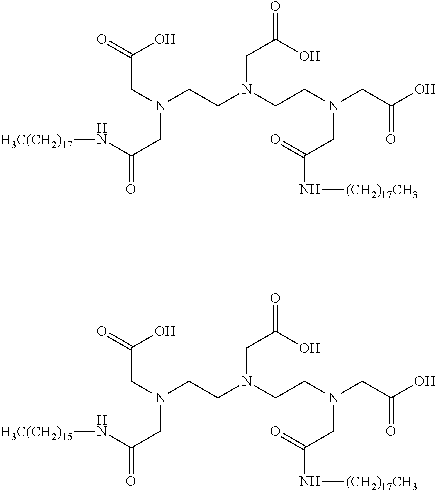

### Example 4

The following examples were prepared by reacting diethylenetriaminepentaacetic acid dianhydride with an alkanol.

To 1.5 g (4.198 mM) of diethylenetriaminepentaacetic acid dianhydride (Sigma; D6148-10g (042K006); CAS23911-26-4; Mw=357.32) suspended in 100 ml of DCM, 2.923 ml (4 eq) of DIEA was added followed by 3.2 ml (16.792 mM; 4 eq, Mw=158.29, d=0.83) of CH₃(CH₂)₉OH. Solution was turbid so additional 30 ml of DMSO was added. Residue dissolved within 1 hr. After 2 hrs reaction, the solvent was evaporated and the product was precipitated with mixture of AcCN/H2O. Yield: 2.1 g.

MA-(C10/C10)diester; C₁₆H₂₉N₅O₈; 5,8-bis(carboxymethyl)-2-(2-(decyloxy)-2-oxoethyl)-10-oxo-11-oxa-2,5,8-triazahenicosane-1-carboxylic acid

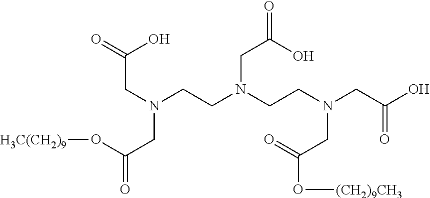

### Example 5

The following example was prepared by reacting diethylenetriaminepentaacetic acid dianhydride with an ethylenediamide.

To 0.3 g (0.446 mmol) of MA-(C10/C₁₀)dialkylamide dissolved in 20 ml of dry DMF, 1.107 g (2.2676 mmol, 6 eq) of HCTU (Mw=413.7), 0.428 g (2.676 mmol, 6 eq) of (N-Boc ethylenediamide, Aldrich, Mw=160.1, d=1.012) and 0.465 ml (2.2676 mmol, 6 eq) of DIEA were added. After 2 hrs reaction mixture was diluted with water and Boc protected product was extracted with 20 ml DCM 3 times. Organic layer was dried with Mg(SO4)2 and DCM was evaporated.

MA-(C10/C10)dialkylamide was isolated using a ISCO COMBIFLASH chromatography separation instrument with a 24 g normal phase silica gel column, 100% DCM for 3 CV (column volume) and 0-100% MeOH for 9 CV, detection 215 nm, flow 40 ml/min.

MA-(C10/C10)diamide-tri(ethylenediamide); 2,2′-(17-(2-(2-aminoethylamino)-2-oxoethyl)-12,22-dioxo-11,14,17,20,23-pentaazatritriacontane-14,20-diyl)bis(N-(2-aminoethyl)acetamide)

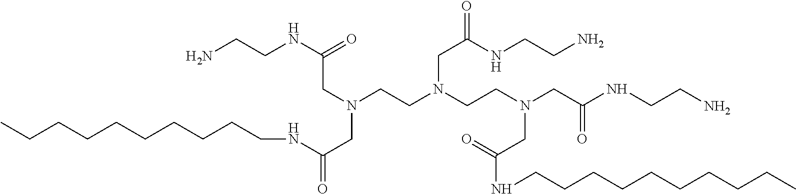

### Example 6

In like fashion as in Example 5 are made the following compounds:
MA-(C12/C12)diamide-tri(ethylenediamide)
MA-(C14/C14)diamide-tri(ethylenediamide)
MA-(C16/C16)diamide-tri(ethylenediamide)
MA-(C18/C18)diamide-tri(ethylenediamide)
MA-(C16/C18)diamide-tri(ethylenediamide).

### Example 7

The following example was prepared by reacting diethylenetriaminepentaacetic acid dianhydride with an iminodiethylamine.

To 0.3 g (0.446 mmol) of MA-(C10/C10)dialkylamide dissolved in 20 ml of dry DMF, 1.107 g (2.2676 mmol, 6 eq) of HCTU (Mw=413.7), 0.905 g (2.676 mmol, 6 eq) of (N1-Boc-2,2-iminodiethylamine, Aldrich, Mw=203.28, d=1.020) and 0.465 ml (2.2676 mmol, 6 eq) of DIEA were added. After 2 hrs reaction mixture was diluted with water and Boc protected product was extracted with 20 ml DCM 3 times. Organic layer was dried with Mg(SO4)2 and DCM was evaporated.

MA-(C10/C10)diamide-tri(iminodiethylamine)was isolated using a ISCO COMBIFLASH chromatography separation instrument with a 24 g normal phase silica gel column, 100% DCM for 4 CV(column volume) and 0-40% MeOH for 4 CV, detection 215 nm, flow 40 ml/min.

MA-(C10/C10)diamide-tri(iminodiethylamine); 2,2′-(17-(2-(2-(2-aminoethylamino)ethylamino)-2-oxoethyl)-12,22-dioxo-11,14,17,20,23-pentaazatritriacontane-14,20-diyl)bis(N-(2-(2-aminoethylamino)ethyl)acetamide)

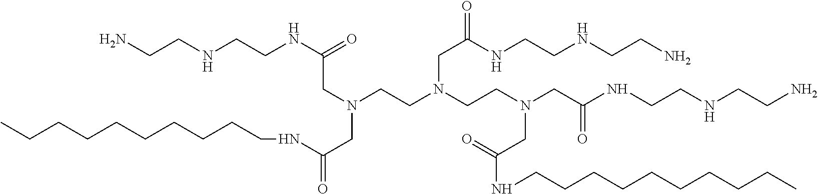

### Example 8

In like fashion as in Example 7 are made the following compounds:
MA-(C12/C12)diamide-tri(iminodiethylamine)
MA-(C14/C14)diamide-tri(iminodiethylamine)
MA-(C16/C16)diamide-tri(iminodiethylamine)

### Example 9

The following example was prepared by reacting diethylenetriaminepentaacetic acid dianhydride with an ethylene glycol amine.

To 0.3 g (0.446 mmol) of MA-(C10/C10)dialkylamide dissolved in 20 ml of dry DMF, 1.107 g (2.2676 mmol, 6 eq) of HCTU (Mw=413.7), 1.05 g (2.676 mmol, 6 eq) of (Trt-NH-(PEG)2-NH2, EMD, Mw=462.6) and 0.465 ml (2.2676 mmol, 6 eq) of DIEA were added. After 2 hrs reaction mixture was diluted with water and Trt protected product was extracted with 20 ml DCM 3 times. Organic layer was dried with Mg(SO4)2 and DCM was evaporated. Trt group was removed by 10% TFA/DCM solvent mixture.

MA-(C10/C10)diamide-tri(PEG2-amine) was isolated using a ISCO COMBIFLASH chromatography separation instrument with a 24 g normal phase silica gel column, 100% DCM for 3 CV(column volume) and 0-100% MeOH for 9 CV, detection 215 nm, flow 40 ml/min. Yield: 0.45 g

MA-(C10/C10)diamide-tri(PEG2-amine); 2,2′-(17-(16-amino-2-oxo-7,10,13-trioxa-3-azahexadecyl)-12,22-dioxo-11,14,17,20,23-pentaazatritriacontane-14,20-diyl)bis(N-(3-(2-(2-(3-aminopropoxy)ethoxy)ethoxy)propyl)acetamide)

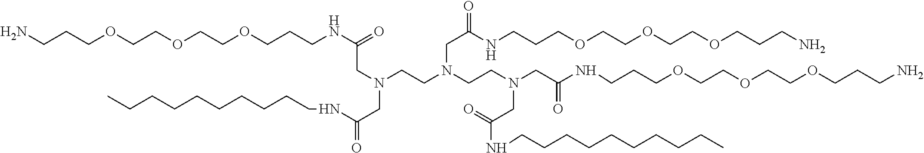

### Example 10

In like fashion are made the following compounds:
MA-(C12/C12)diamide-tri(PEG2-amine)
MA-(C14/C14)diamide-tri(PEG2-amine)
MA-(C16/C16)diamide-tri(PEG2-amine).

### Example 11

The following example was prepared by reacting diethylenetriaminepentaacetic acid dianhydride with an ethylene glycol amine.

To 0.3 g (0.446 mmol) of MA-(C10/C10)dialkylamide dissolved in 20 ml of dry DMF, 1.107 g (2.2676 mmol, 6 eq) of HCTU (Mw=413.7), 0.462 g (2.676 mmol, 6 eq) of (m-dPEG-amide, Pept. Int, NH₂(CH₂)₂[O(CH₂)₂]₃OCH₃, Mw=207.27) and 0.465 ml (2.2676 mmol, 6 eq) of DIEA were added. After 2 hrs reaction mixture was diluted with water and Trt protected product was extracted with 20 ml DCM 3 times. Organic layer was dried with Mg(SO4)2 and DCM was evaporated.

MA-(C10/C10)diamide-tri(PEG3-methoxy) was isolated using a ISCO COMBIFLASH chromatography separation instrument with a 24 g normal phase silica gel column, 100% DCM for 4 CV(column volume) and 0-50% MeOH for 4.5 CV, detection 215 nm, flow 40 ml/min. Yield: 0.5 g.

MA-(C10/C10)diamide-tri(PEG3-methoxy); 2,2′-(12,22-dioxo-17-(15-oxo-2,5,8,11-tetraoxa-14-azahexadecan-16-yl)-11,14,17,20,23-pentaazatritriacontane-14,20-diyl)bis(N-(2,5,8,11-tetraoxamidecan-13-yl)acetamide)

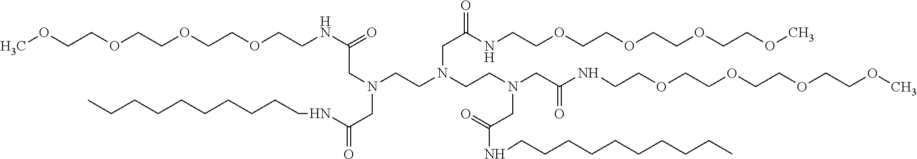

### Example 12

In like fashion are made the following compounds:
MA-(C12/C12)diamide-tri(PEG3-methoxy)
MA-(C14/C14)diamide-tri(PEG3-methoxy)
MA-(C16/C16)diamide-tri(PEG3-methoxy).

### Example 13

Compounds of this disclosure include the formulas:

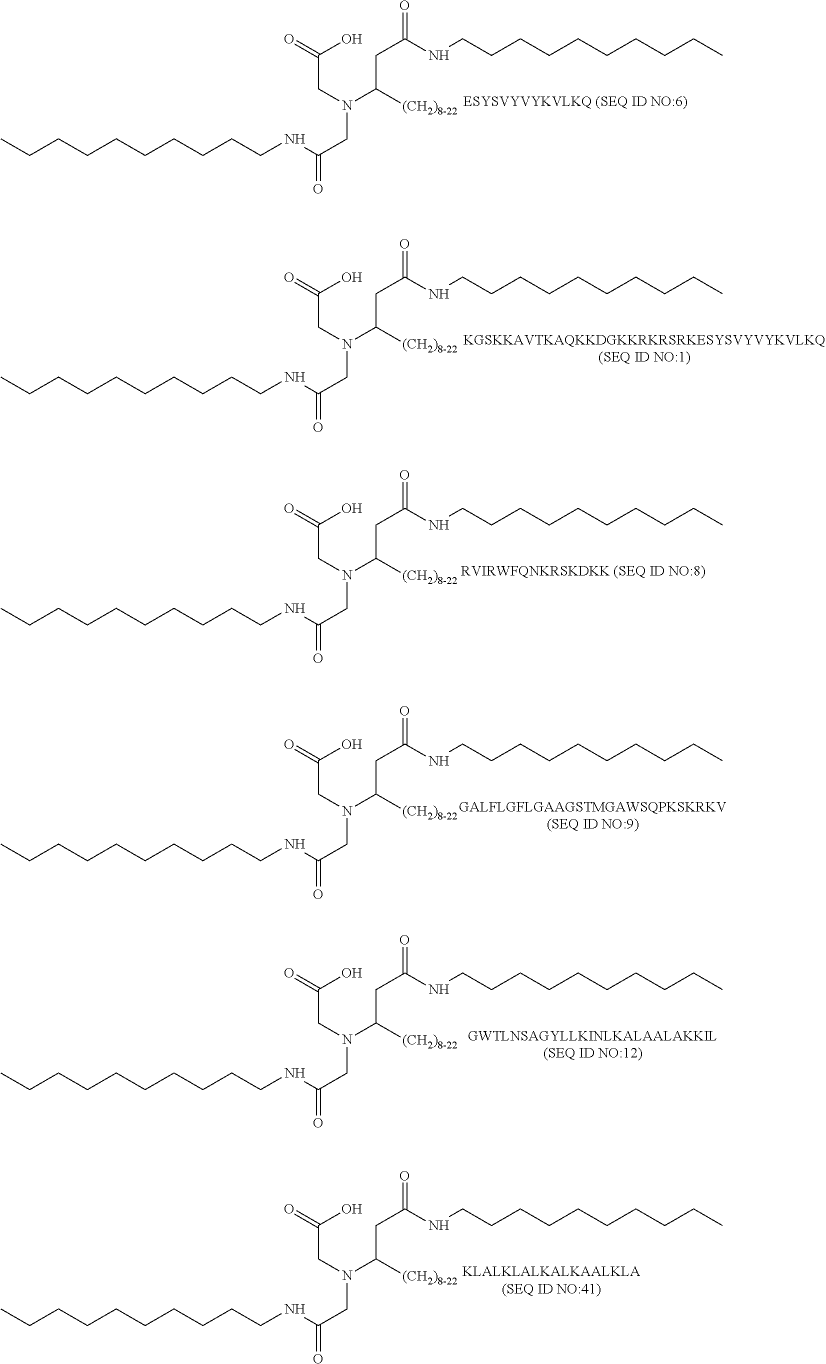

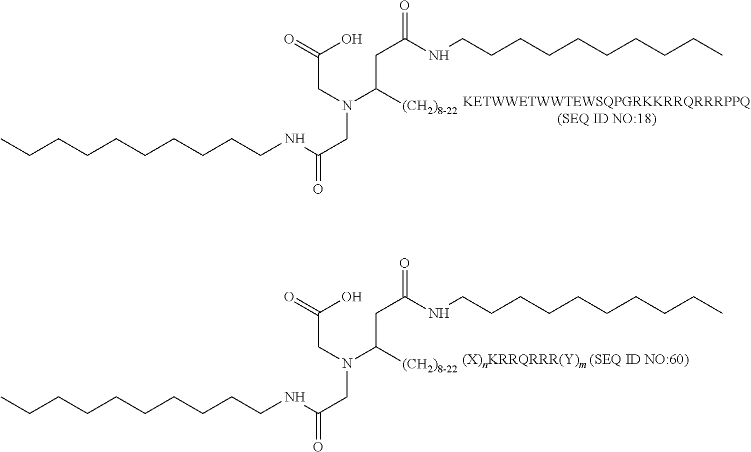

In the formula above, X and Y are independently, for each occurrence, an amino acid residue, and n is from 0 to 50 and m is from 0 to 50.

### Example 14

Compounds of this disclosure include the formulas:

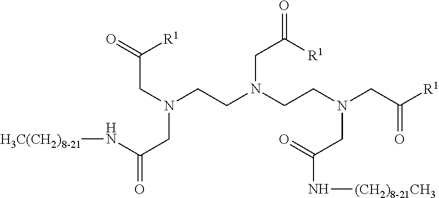

R¹ may be independently, for each occurrence:

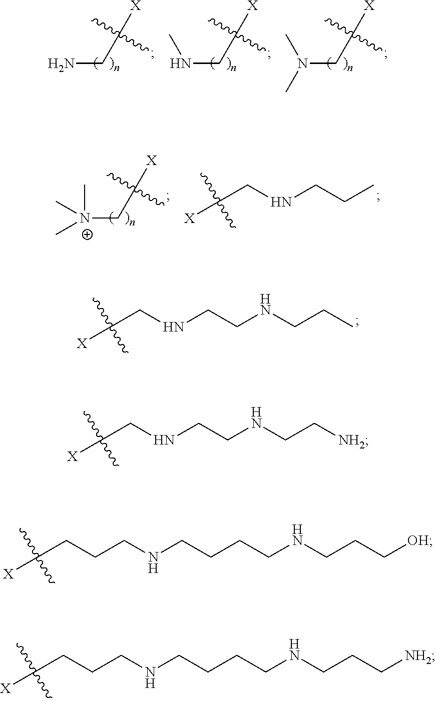

halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano.

In the formulas above, X represents the atom of the functional group that is directly attached to the multi-arm amine compound. In some embodiments, X may be a carbon, nitrogen, or oxygen atom. In the structures above n is from 1 to 22; or from 1 to 18; or from 1 to 16; or from 1 to 10; or from 1 to 5 (or 1, 2, 3, 4, 5, 6, 7, 8, 9, 10, 11, 12, 13, 14, 15, 16, 17, 18, 19, 20, 21, or 22).

Further example compounds of this disclosure include the formulas:

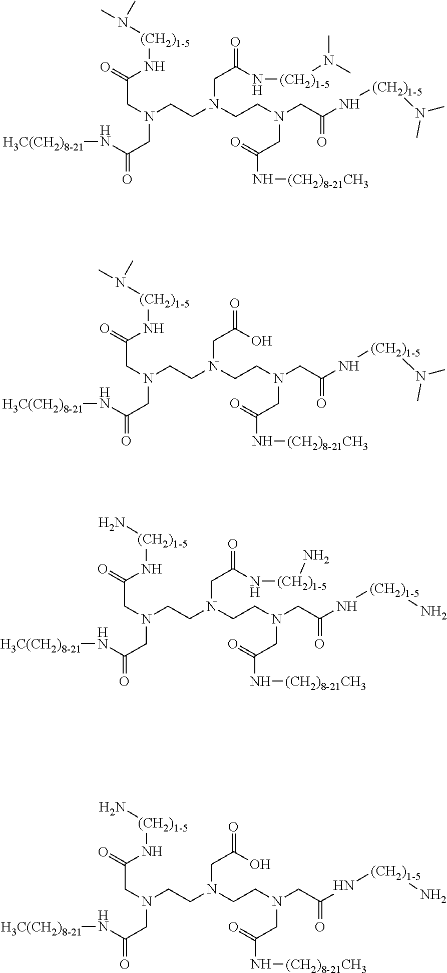

### Example 15

Compounds of this disclosure include the formulas:

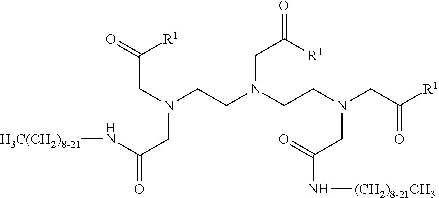

R¹ may be independently, for each occurrence:

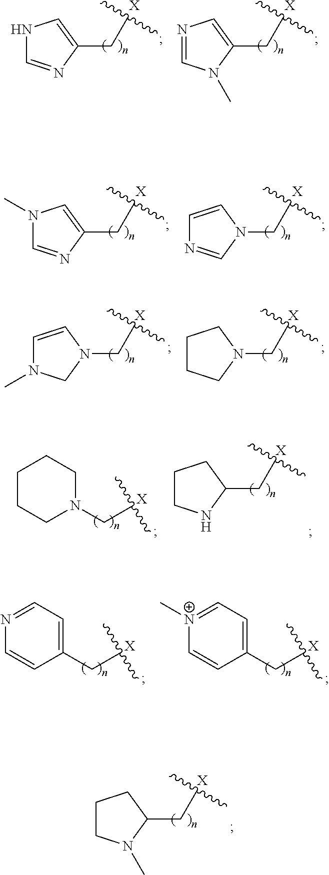

a natural or non-naturally occurring amino acid residue; halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, alkoxy, aryloxy, alkylthio, sulfonyl, ketone, aldehyde, ester, heterocyclyl, aryl, heteroaryl, fluoroalkyl, or cyano.

In the formulas above, X represents the atom of the tail that is directly attached to the multi-arm amine compound. In some embodiments, X may be a carbon, nitrogen, or oxygen atom. In the structures above n is from 1 to 22; or from 1 to 18; or from 1 to 16; or from 1 to 10; or from 1 to 5 (or 1, 2, 3, 4, 5, 6, 7, 8, 9, 10, 11, 12, 13, 14, 15, 16, 17, 18, 19, 20, 21, or 22).

### Example 16

Compounds of this disclosure include the formulas:

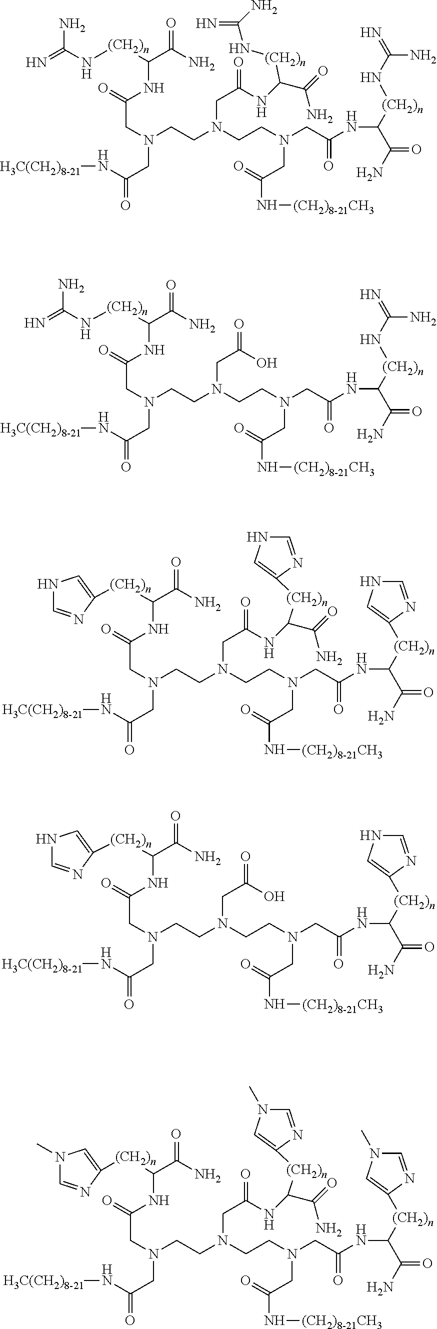

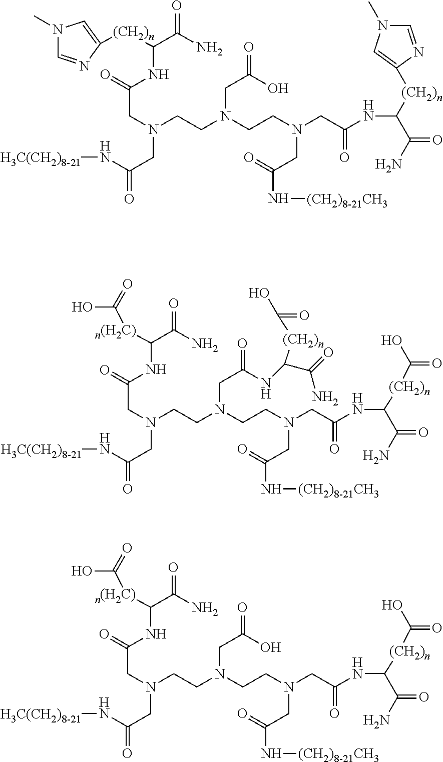

In the formulas above, n is from 1 to 22; or from 1 to 18; or from 1 to 16; or from 1 to 10; or from 1 to 5 (or 1, 2, 3, 4, 5, 6, 7, 8, 9, 10, 11, 12, 13, 14, 15, 16, 17, 18, 19, 20, 21, or 22).

### Example 17

Compounds of this disclosure include the formulas:

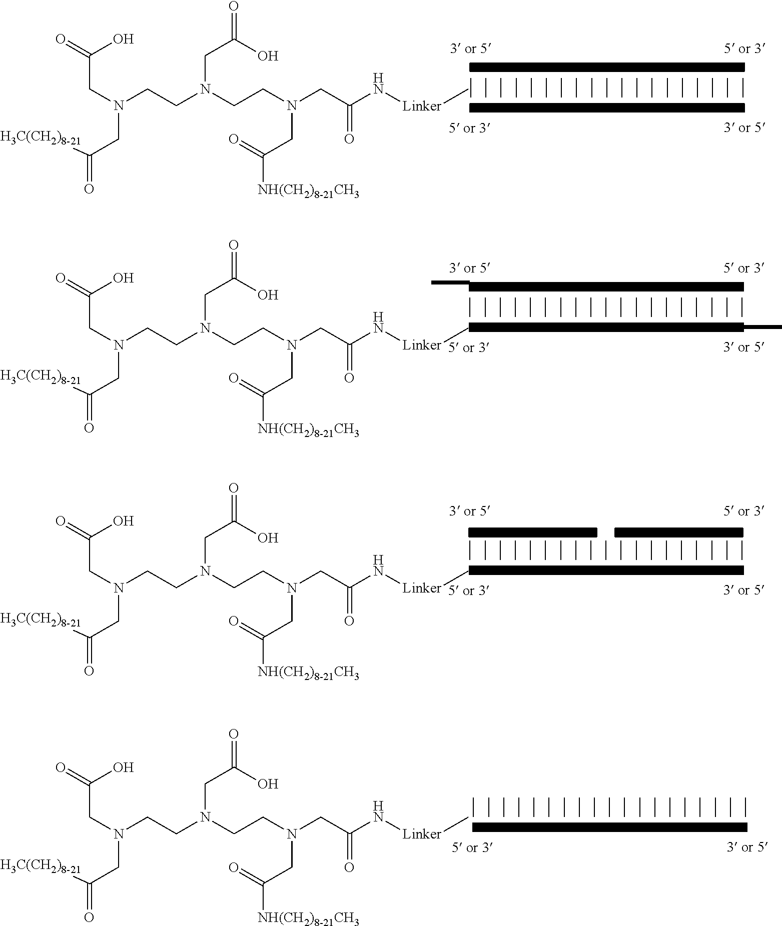

In the formulas above, the linker group is an organic linker consisting of 1-40 atoms selected from hydrogen, carbon, oxygen, nitrogen, and sulfur atoms. The multi-arm amine compounds as shown are linked to a double-stranded RNA (dsRNA) molecule or single-stranded RNA molecule. The dsRNA molecule may have two blunt ends or two 3′-end overhangs. Further, the dsRNA molecule may be an mdRNA molecule. The length and number of base pairs of the RNA molecules is illustrative and not limiting. For example, the number of base pairs of the dsRNA molecules may be from 15 to 40 base pairs or from 18 to 30 base pairs. A single-stranded RNA molecule may be from 10 to 50 nucleobases or from 12 to 30 nucleobases.

Representative multi-arm amine compounds with at least one RNA molecule attached via a disulfide linker include:

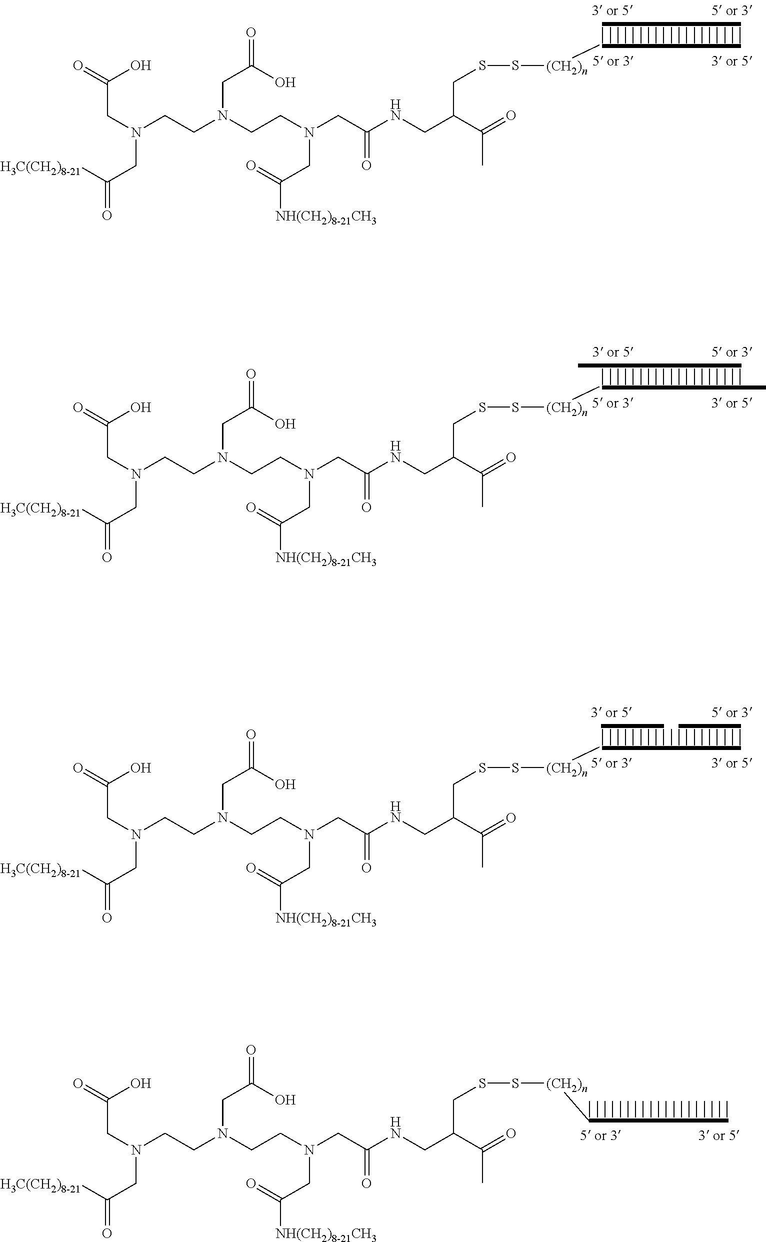

In the structures above, n is from 1 to 22; or from 1 to 18; or from 1 to 16; or from 1 to 10; or from 1 to 5 (or 1, 2, 3, 4, 5, 6, 7, 8, 9, 10, 11, 12, 13, 14, 15, 16, 17, 18, 19, 20, 21, or 22).

### Example 18

### Intracellular Delivery of Interfering RNA with Multi-Arm Amine Formulations

This example demonstrates the use of formulations containing multi-arm amines to provide gene silencing using an siRNA. Transfection with a multi-arm amine containing formulation was performed in A549 cells and 9L/LacZ cells.

9L/LacZ cells, a cell line constitutively expressing β-galactosidase, were transfected with multi-arm amine containing formulations. 9L/LacZ cells are rat gliosarcoma fibroblast cells and were obtained from ATCC (#CRL-2200). 9L/LacZ cells were grown in Dulbecco's Modified Essential Medium (DMEM) media with a supplement of 1 mM sodium pyruvate, nonessential amino acids, and 20% fetal bovine serum. Cells were cultured at 37° C. and 5% CO₂ supplemented with an antibiotic mixture containing 100 units/ml penicillin, 100 μg/ml streptomycin and 0.25 mg/ml Fungizone (Invitrogen).

One day prior to transfection, 9L/LacZ cells were plated at 6,000 cells/well on a 96-well plate (75 μl/well). On the day of transfection, the cell culture medium was replaced with 75 μl of no-serum OPTIMEM and 25 μl of the transfection formulation. The cells were incubated with the transfection formulation for five hours followed by the addition of 100 μL of cell culture medium containing 10% serum (final serum was 5%). One day post-transfection, the cell culture medium was replaced with 100 μL containing 10% serum. Two day later, the cells were lysed and β-galactosidase activity assayed and protein levels were measured to normalize β-galactosidase activity levels.

A549 cells are a carcinoma human alveolar basal epithelial cell line. One day prior to transfection A549 cells were plated at 7,500 cells/well on a 96-well plate (75 μl/well). The transfection protocol described above for 9L/LacZ cells was used for A549 cell transfections. Target gene levels were measured by qRT-PCR (Qiagen). The manufacturer's protocol was followed.

RNAIMAX (Invitrogen) served as a positive control transfection reagent. Qneg (Qiagen) represents a random ribonucleic acid sequence and served as a negative control of siRNA mediated gene silencing. The knockdown activity of the LacZ specific siRNA (transfection of 9L/LacZ cells) and the PPIB specific siRNA (transfection of A549 cells) is shown as a percentage of the level of either the LacZ activity after transfection of 9L/LacZ cells with the control Qneg siRNA or the mRNA levels of the target gene (PPIB) after transfection of A549 cells with the control Qneg siRNA. Each siRNA was transfected at a concentration of 100 nM. The nucleotide sequence of the sense and antisense strands of the LacZ siRNA and PPIB siRNA are as follows:

LacZ siRNA:
(SEQ ID NO: 42)
Sense
5′- CUACACAAAUCAGCGAUUUTT -3′
(SEQ ID NO: 43)
Antisense
5′- AAAUCGCUGAUUUGUGUAGT_{d}C -3′
PPIB siRNA:
(SEQ ID NO: 44)
Sense
5′- GGAAAGACUGUUCCAAAAAUU -3′
(SEQ ID NO: 45)
Antisense
5′- UUUUUGGAACAGUCUUUCCUU -3′

The individual multi-arm amines MA-(C10/C10)dialkylamide, MA-(C12/C12)dialkylamide, and MA-(C16/C16)dialkylamide were complexed with 100 nM siRNA and mixed with a binary lipid transfection formulation. The lipids used were cholesterol and the amino acid lipid C12-norArg-C12 which is described in U.S. patent application Ser. No. 12/114,284.

The transfection formulations and corresponding percent knockdown activity for each siRNA are shown in Table 1. The molar percent of each formulation lipid component is shown as a percentage of total lipid.

Also, “(C10)2-DTPA” refers to MA-(C10/C10)dialkylamide, “(C12)2-DTPA” refers to MA-(C12/C12)dialkylamide, and “(C16)2-DTPA” refers to MA-(C16/C16)dialkylamide. Each formulation was prepared in 10 mM HEPES and 5% dextrose. The final pH of each formulation was pH 7.2 and the final N/P ratio was 1.8.

TABLE 1
Gene Silencing Knockdown Using Multi-arm amine Delivery Compounds

| | % Knockdown vs. Qneg | |
|---|---|---|
| Formulation (Mol %) | 9L/LacZ | PPIB A549 |
| RNAIMAX | 84 | 90 |
| C12-norArg-C12/cholesterol (50:50) | 58 | 67 |
| MA-(C10/C10)dialkylamide/C12-norArg-C12/cholesterol (1/50/49) | 71 | 81 |
| MA-(C10/C10)dialkylamide/C12-norArg-C12/cholesterol (2:50:48) | 70 | 75 |
| MA-(C10/C10)dialkylamide/C12-norArg-C12/cholesterol (4:50:46) | 68 | 80 |
| MA-(C10/C10)dialkylamide/C12-norArg-C12/cholesterol (8:50:42) | 61 | 82 |
| MA-(C10/C10)dialkylamide/C12-norArg-C12/cholesterol (16:50:34) | 78 | 96 |
| MA-(C10/C10)dialkylamide/C12-norArg-C12/cholesterol (32:50:18) | −28 | 45 |
| MA-(C12/C12)dialkylamide/C12-norArg-C12/cholesterol (1:50:49) | 73 | 79 |
| MA-(C12/C12)dialkylamide/C12-norArg-C12/cholesterol (2:50:48) | 73 | 80 |
| MA-(C12/C12)dialkylamide/C12-norArg-C12/cholesterol (4:50:46) | 66 | 79 |
| MA-(C12/C12)dialkylamide/C12-norArg-C12/cholesterol (8:50:42) | 59 | 79 |
| MA-(C12/C12)dialkylamide/C12-norArg-C12/cholesterol (16:50:34) | 82 | 88 |
| MA-(C12/C12)dialkylamide/C12-norArg-C12/cholesterol (32:50:18) | 63 | 87 |
| MA-(C16/C16)dialkylamide/C12-norArg-C12/cholesterol (1:50:49) | 76 | 76 |
| MA-(C16/C16)dialkylamide/C12-norArg-C12/cholesterol (2:50:48) | 77 | 83 |
| MA-(C16/C16)dialkylamide/C12-norArg-C12/cholesterol (4:50:46) | 77 | 79 |
| MA-(C16/C16)dialkylamide/C12-norArg-C12/cholesterol (8:50:42) | 68 | 75 |
| MA-(C16/C16)dialkylamide/C12-norArg-C12/cholesterol (16:50:34) | 52 | 64 |
| MA-(C16/C16)dialkylamide/C12-norArg-C12/cholesterol (32:50:18) | 23 | 41 |

As shown in Table 1, transfections performed with formulations containing a multi-arm amine compound of this disclosure provided gene silencing knockdown activity for LacZ of up to 82%, and for PPIB of up to 96%.

As shown in Table 1, transfections performed with RNAIMAX provided gene silencing knockdown activity for LacZ of 84%, and for PPIB of 90%.

As shown in Table 1, transfections performed with the binary lipid formulation C12-norArg-C12/cholesterol (50:50) provided gene silencing knockdown activity for LacZ of 58%, and for PPIB of 67%.

These data indicate that the multi-arm amine compounds of this disclosure provide intracellular delivery of an interfering ribonucleic acid (siRNA) and gene silencing knockdown activity.

### Example 15

### Permeation of PTH Across an Epithelial Cell Layer In Vitro

The permeation of parathyroid hormone (PTH, teriparatide) across an epithelial cell layer (EPIAIRWAY Tissue Model) in the presence of a multi-arm amine compound of this disclosure was measured. Further, transepithelial resistance (TER assay), cell viability (MTT), and in certain instances cell cytotoxicity (LDH assay) of an epithelial cell layer in the presence of a multi-arm amine compound of this disclosure was measured.

Table 2 shows the PTH/multi-arm amine formulations and control formulations used to measure PTH permeation kinetics. The pH of these formulations was 7.

TABLE 2
PTH formulations

| Formulation | Composition (each formulation containing |
|---|---|
| No. | 3 mg/mL PTH₁₋₃₄) |
| 11 | 36 mg/mL Sorbitol |
| | 1.85 mg/mL MA-(C10/C10)dialkylamide |
| 12 | 36 mg/mL Sorbitol |
| | 3.7 mg/mL MA-(C10/C10) dialkylamide |
| 13 | 36 mg/mL Sorbitol |
| | 5 mg/mL Chlorobutanol |
| | 1.85 mg/mL MA-(C10/C10) dialkylamide |
| 14 | 36 mg/mL Sorbitol |
| | 5 mg/mL Chlorobutanol |
| | 3.7 mg/mL MA-(C10/C10) dialkylamide |
| 31 | 0.3% Triton ® X-100 |
| 32 | Air-100 Media (MaTek) |

In Table 2, Formulation 32 served as a negative control for TER, MTT and permeation assays, and Formulation 31 served as a positive control.

Permeation kinetic measurements were taken 60 minutes post-treatment. Briefly, for PTH permeation, basolateral media was collected and analyzed by RP-HPLC for PTH content. Permeation was presented as the percent of PTH that crossed from the apical side of the epithelial cell monolayer to the basolateral cell surface. Cell viability was measured by an MTT assay and presented as a percent. For the purpose of this assay, media alone (negative control; Formulation 1) represented 100% cell viability. Thus, a lower percent MTT indicates a negative effect on cell viability. TER was calculated as described above. For this Example, TER was presented as a percent with a lower percent indicating a greater reduction in TER.

The permeation kinetics of the formulations described in Table 2 are summarized in Table 3. The data shown in Table 3 indicated that the multi-arm amine compounds of this disclosure provided significant epithelial cell layer permeation of the biological agent PTH while maintaining cell viability.

TABLE 3
Permeation of PTH with Multi-arm amines

| Formulation | Permeation % | | MTT % | | TER % | |
|---|---|---|---|---|---|---|
| No. | Average | Stdev. | Average | Stdev. | Average | Stdev. |
| 11 | 1 | 0.33 | 75.73 | 6.21 | 13.50 | 4.97 |
| 12 | 3.41 | 0.73 | 55.55 | 4.44 | 3.83 | 0.54 |
| 13 | 3.38 | 0.23 | 61.40 | 2.33 | 3.79 | 1.14 |
| 14 | 4.55 | 0.83 | 27.27 | 6.83 | 2.13 | 0.09 |
| 31 | 6.57 | 1.86 | 1.17 | 0.15 | 3.22 | 0.29 |
| 32 | 0.11 | 0.39 | 100 | 0.88 | 115.19 | 0.64 |

As shown in Table 3, the MA-(C10/C10) dialkylamide-containing Formulations 11-14 provided from about 1% PTH permeation to about 4.6% PTH permeation. Further, Formulations 11-14 exhibited substantially reduced TER levels. MTT measurements indicated that the multi-arm amine formulations containing PTH maintained cell viability.

In addition, the MA-(C10/C10) dialkylamide-containing formulations had either 1.85 mg/mL or 3.7 mM MA-(C10/C10) dialkylamide, and the level of permeation increased as the concentration of multi-arm amine increased.

As shown in Table 3, Formulation 32 (media with PTH) provided little to no permeation (0.11%), little to no TER reduction (115.19%) and no effect on cell viability. Formulation 31 (Triton X100™ with PTH; positive control) provided about 6.6% permeation and reduced TER levels to about 3%, but also greatly reduced cell viability (MTT of 1.17%).

Methods and procedures used to measure the permeation kinetics are described below. Permeation kinetics measured include transepithelial electrical resistance (TER), cytotoxicity (LDH), cell viability (MTT) and epithelial cell layer permeation of a biological agent. The cell culture conditions and protocols for each assay are explained below in detail.

Cell cultures: Normal, human-derived tracheal/bronchial epithelial cells served as the model cell system for measuring permeation kinetics. The cells were supplied by MatTek Corp. (Ashland, Mass.) as the EpiAirway™ Tissue Model. The cells were provided as a confluent monolayer on a Millipore Milicell-CM cell culture insert with a pore size of 0.4 μM, inner diameter of 0.8 cm and surface area of 0.6 cm² and comprised of transparent hydrophilic Teflon (PTFE). Upon receipt, the membranes were cultured in 1 ml basal media (phenol red-free and hydrocortisone-free Dulbecco's Modified Eagle's Media (DMEM) at 37° C./5% CO₂ for 24-48 hours before use. Inserts were fed daily.

Transepithelial Electrical Resistance (TER): TER measurements were taken using the Endohm-12 Tissue Resistance Measurement Chamber connected to the EVOM Epithelial Voltohmmeter (World Precision Instruments, Sarasota, Fla.) with the electrode leads. The electrodes and a tissue culture blank insert were equilibrated for at least 20 minutes in MatTek™ media with the power off prior to checking calibration. The background resistance was measured with 1.5 ml media in the Endohm tissue chamber and 300 μl media in the blank insert. The top electrode was adjusted so that it was close to, but not making contact with, the top surface of the insert membrane. Background resistance of the blank insert was about 5-20 ohms. For each TER determination, about 300 μl of MatTek™ media was added to the insert followed by placement in the Endohm chamber. TER values are a function of the surface area of the tissue. An example of how TER was calculated is as follows:

$$Nominal Resistance , Ohm * {cm}^{2} = ( TERt - blank ) * 0.12$$

$$Relative TER , \% = \frac{TERt - blank}{TER0 - blank} \times 100$$

Where transepithelial electrical resistance at time t=TERₜ and blank refers to the TER of an empty insert. By this method of calculation, TER will be expressed as both Ohms*cm2 and percent original TER value.

TER recovery was calculated as described in the above paragraph.

Cell Viability (MTT Assay): Cell viability was assessed using the MTT assay (MTT-100, MatTek kit). This kit measures the uptake and transformation of tetrazolium salt to formazan dye. Thawed and diluted MTT concentrate was prepared 1 hour prior to the end of the dosing period by mixing 2 mL of MTT concentrate with 8 mL of MTT diluent. Each cell culture insert was washed twice with PBS containing Ca⁺² and Mg⁺² and then transferred to a new 96-well transport plate containing 100 μl of the mixed MTT solution per well. This 96-well transport plate was incubated for three hours at 37° C. and 5% CO₂. After the three hour incubation, the MTT solution was removed and the cultures were transferred to a second 96-well feeder tray containing 250 μL MTT extractant solution per well. An additional 150 μl of MTT extractant solution was added to the surface of each culture well and the samples sat at room temperature in the dark for a minimum of two hours and maximum of 24 hours. The insert membrane was then pierced with a pipet tip and the solutions in the upper and lower wells were allowed to mix. Two hundred microliters of the mixed extracted solution along with extracted blanks (negative control) was transferred to a 96-well plate for measurement with a microplate reader. The optical density (OD) of the samples was measured at 570 nm with the background subtraction at 650 nm on a plate reader. Cell viability was expressed as a percentage and calculated by dividing the OD readings for treated inserts by the OD readings for the PBS treated inserts and multiplying by 100. For the purposes of this assay, it was assumed that PBS had no effect on cell viability and therefore represented 100% cell viability.

Cytotoxicity (LDH Assay): The amount of cell death was assayed by measuring the loss of lactate dehydrogenase (LDH) from the cells using a CytoTox 96 Cytotoxicity Assay Kit (Promega Corp., Madison, Wis.). A treatment of 1% Octylphenolpoly(ethyleneglycolether)×(Triton X-100™) diluted in PBS was used as a lysis control. One percent Triton X-100™ mediated cell lysis was normalized to 100%. For basal-lateral LDH levels, triplicates of 50 μl of the basal media were loaded into a 96-well assay plate. For apical LDH levels, 150 μl of Epi-Cm was added to the apical side of each chamber and mixed by pipeting. One hundred and fifty microliters was then removed and diluted 2-fold prior to performing the LDH assay. All apical LDH assay were performed in triplicate and with 50 μl of the diluted test solution. Fresh, cell-free culture medium was used as a blank. Total LDH levels were determined by lysing cells in a final concentration of 0.9% Triton-X100™. Fifty microliters of substrate solution was added to each well and the plates incubated for 30 minutes at room temperature in the dark. Following incubation, 50 μl of stop solution was added to each well and the plates read on an optical density plate reader at 490 nM. Cytotoxicity was expressed as a percentage calculated by subtracting the average absorbance of the PBS control wells as the endogenously released LDH level and expressing that value relative to the average Triton-X100™ control, which represents total LDH content.

$$Relative Cytotoxicity , \% = \frac{ODx - ODpbs}{ODtriton} \times 100$$

Peptide Concentration: The amount of PTH₁₋₃₄ in the permeation assay was measured by reverse phase high pressure liquid chromatography (RP-HPLC; Agilent HPLC model 1100). Briefly, PTH₁₋₃₄ standards in the range of 1.56 μg/mL to 100 μg/mL were prepared with MatTek media to calculate the concentration of PTH₁₋₃₄ for each permeation assay performed. The parameters for the RP-HPLC were as follows:
Sample injection volume: 25 μl
Column: 5 μM, 300A°, Vydac C18, 150 mm×4.6 mm (Western Analytical, p/n: 218TP54, S/N E040408-4-1)
MPA: 90% HPLC grade water+10% acetonitrile+0.1% TFA
MPB: 25% HPLC grade water+75% acetonitrile+0.1% TFA
Flow Rate: 2 ml/minute; stop at 6 minutes
Gradient: Time/Flow rate (mL/min)/% B was 0/2/5; 2/2/60; 3/2/5; 6/2/5.
Detection Time: about 2.5 minutes

The amount of insulin in the permeation assay was measured by ELISA (MILLIPORE EZH1-14K).

What is claimed is:

1. A compound comprising the structure shown in Formula I: 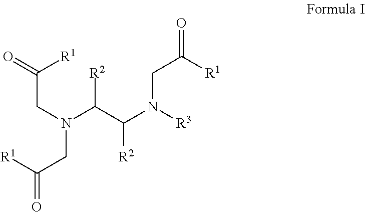
wherein
R¹ are independently, for each occurrence, selected from
—OR⁴, —O(CH₂)ₙOR⁴, —O(CH₂CH₂O)ₗR⁴, —O(CH₂CH₂NH)ₘR⁴, —O(CH₂)ₙNR⁴R⁵;
—NHR⁴, —NR⁴R⁵, —NH(CH₂CH₂O)ₗR⁴, —NH(CH₂CH₂NH)ₘR⁴, —NH(CH₂)ₙNR⁴R⁵, —NH(CH₂CH₂NH)ₘ(CH₂)ₙNR⁴R⁵, —NH(CH₂CH₂O)ₗ(CH₂)ₙNR⁴R⁵, —NH(CH₂CH₂O)ₗNR⁴R⁵, —NH(CH₂CH₂NH)ₘ(CH₂)ₙOR⁴;
a nucleic acid;
a peptide comprising 2-50 amino acid residues;
a D- or L-amino acid residue having the formula —NR^{N}—CR⁸R⁹—(C═O)—NR⁴R⁵ or —NR^{N}—CR⁸R⁹—(C═O)—OH, wherein
R⁸ is independently, for each occurrence, a substituted or unsubstituted side chain of an amino acid;
R⁹ is independently, for each occurrence, hydrogen, or an organic group consisting of carbon, oxygen, nitrogen, sulfur, and hydrogen atoms, and having from 1 to 20 carbon atoms, or C(1-5)alkyl, cycloalkyl, cycloalkylalkyl, C(3-5)alkenyl, C(3-5)alkynyl, C(1-5)alkanoyl, C(1-5)alkanoyloxy, C(1-5)alkoxy, C(1-5)alkoxy-C(1-5)alkyl, C(1-5)alkoxy-C(1-5)alkoxy, C(1-5)alkyl-amino-C(1-5)alkyl-, C(1-5)dialkyl-amino-C(1-5)alkyl-, nitro-C(1-5)alkyl, cyano-C(1-5)alkyl, aryl-C(1-5)alkyl, 4-biphenyl-C(1-5)alkyl, carboxyl, or hydroxyl,
R^{N} is independently, for each occurrence, hydrogen, or an organic group consisting of carbon, oxygen, nitrogen, sulfur, and hydrogen atoms, and having from 1 to 20 carbon atoms, or C(1-5)alkyl, cycloalkyl, cycloalkylalkyl, C(3-5)alkenyl, C(3-5)alkynyl, C(1-5)alkanoyl, C(1-5)alkanoyloxy, C(1-5)alkoxy, C(1-5)alkoxy-C(1-5)alkyl, C(1-5)alkoxy-C(1-5)alkoxy, C(1-5)alkyl-amino-C(1-5)alkyl-, C(1-5)dialkyl-amino-C(1-5)alkyl-, nitro-C(1-5)alkyl, cyano-C(1-5)alkyl, aryl-C(1-5)alkyl, 4-biphenyl-C(1-5)alkyl, carboxyl, or hydroxyl;
wherein
R⁴ and R⁵ are independently, for each occurrence, hydrogen or a substituted or unsubstituted C(1-22)alkyl having 1, 2, 3, 4, 5, 6, 7, 8, 9, 10, 11, 12, 13, 14, 15, 16, 17, 18, 19, 20, 21, or 22 carbon atoms or C(2-22)alkenyl having 2, 3, 4, 5, 6, 7, 8, 9, 10, 11, 12, 13, 14, 15, 16, 17, 18, 19, 20, 21, or 22 carbon atoms;
wherein R⁴ and R⁵ of —NR⁴R⁵ optionally form, together with the nitrogen atom of —NR⁴R⁵ to which they are attached, a saturated or unsaturated heterocyclic group optionally comprising one or more heteroatoms selected from oxygen, nitrogen and sulfur;
R² are independently, for each occurrence, selected from
hydrogen, C(1-6)alkyl, —(CH₂)ₙaryl, —(CH₂)ₙ(C₆H₄)R⁶, —(CH₂)ₙ(C₆H₄)(CH₂)ₙNR⁴R⁵;
wherein R⁶ is hydrogen, —OR⁴, —NR⁴R⁵; —O(CH₂)ₙOR⁴, —O(CH₂)ₙNR⁴R⁵, —(CH₂CH₂O)ₗR⁴; or —(CH₂CH₂NH)ₘR⁴;
R³ is selected from the group consisting of hydrogen, —(CH₂)ₙCOR¹, and 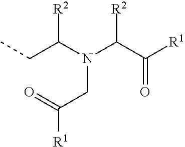
wherein
n is 1 to 22;
l is 1 to 50;
m is 1 to 50;
and salts thereof.

2. The compound of claim 1, wherein two or more of the R¹ contain 8, 9, 10, 11, 12, 13, 14, 15, 16, 17, 18, 19, 20, 21, or 22 carbon atoms.

3. The compound of claim 1, wherein two of the R¹ contain 10 or more carbon atoms and the remaining R¹ are —OH, R² are hydrogen, and R³ is

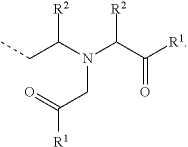

4. A compound comprising the structure shown in Formula I: 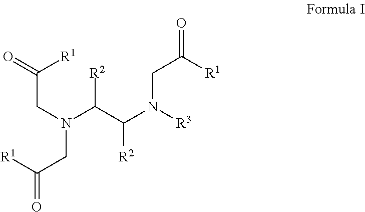
wherein
R¹ are independently, for each occurrence, selected from
—NHR⁴, —NR⁴R⁵, —NH(CH₂CH₂O)ₗR⁴, —NH(CH₂CH₂NH)ₘR⁴, —NH(CH₂)ₙNR⁴R⁵, —NH(CH₂CH₂NH)ₘ(CH₂)ₙNR⁴R⁵, —NH(CH₂CH₂O)ₗ(CH₂)ₙNR⁴R⁵, —NH(CH₂CH₂O)ₗNR⁴R⁵, —NH(CH₂CH₂NH)ₘ(CH₂)ₙOR⁴;
wherein
R⁴ and R⁵ are independently, for each occurrence, hydrogen or a substituted or unsubstituted C(1-22)alkyl having 1, 2, 3, 4, 5, 6, 7, 8, 9, 10, 11, 12, 13, 14, 15, 16, 17, 18, 19, 20, 21, or 22 carbon atoms or C(2-22)alkenyl having 2, 3, 4, 5, 6, 7, 8, 9, 10, 11, 12, 13, 14, 15, 16, 17, 18, 19, 20, 21, or 22 carbon atoms;
wherein R⁴ and R⁵ of —NR⁴R⁵ optionally form, together with the nitrogen atom of —NR⁴R⁵ to which they are attached, a saturated or unsaturated heterocyclic group optionally comprising one or more heteroatoms selected from oxygen, nitrogen and sulfur;
R² are independently, for each occurrence, selected from
hydrogen, C(1-6)alkyl, —(CH₂)ₙaryl, —(CH₂)ₙ(C₆H₄)R⁶, —(CH₂)ₙ(C₆H₄)(CH₂)ₙNR⁴R⁵;
wherein R⁶ is hydrogen, —OR⁴, —NR⁴R⁵; —O(CH₂)ₙOR⁴, —O(CH₂)ₙNR⁴R⁵, —(CH₂CH₂O)ₗR⁴; or —(CH₂CH₂NH)ₘR⁴;
R³ is selected from the group consisting of hydrogen, —(CH₂)ₙCOR¹, and 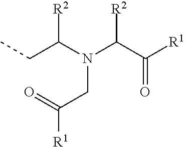
wherein
n is 1 to 22;
l is 1 to 50;
m is 1 to 50;
and salts thereof.

5. The compound according to claim 1, wherein the compound is selected from the group consisting of
2-[2-(bis(carboxymethyl)amino)ethyl-[2-[carboxymethyl-[2-(decylamino)-2-oxoethyl]amino]ethyl]amino]acetic acid;
2-[2-(bis(carboxymethyl)amino)ethyl-[2-[carboxymethyl-[2-(dodecylamino)-2-oxoethyl]amino]ethyl]amino]acetic acid;
2-[2-(bis(carboxymethyl)amino)ethyl-[2-[carboxymethyl-[2-(undecylamino)-2-oxoethyl]amino]ethyl]amino]acetic acid;
2-[2-(bis(carboxymethyl)amino)ethyl-[2-[carboxymethyl-[2-(tridecylamino)-2-oxoethyl]amino]ethyl]amino]acetic acid;
2-[2-(bis(carboxymethyl)amino)ethyl-[2-[carboxymethyl-[2-(tetradecylamino)-2-oxoethyl]amino]ethyl]amino]acetic acid;
2-[2-(bis(carboxymethyl)amino)ethyl-[2-[carboxymethyl-[2-(pentadecylamino)-2-oxoethyl]amino]ethyl]amino]acetic acid;
2-[2-(bis(carboxymethyl)amino)ethyl-[2-[carboxymethyl-[2-(hexadecylamino)-2-oxoethyl]amino]ethyl]amino]acetic acid;
2-[2-(bis(carboxymethyl)amino)ethyl-[2-[carboxymethyl-[2-(heptadecylamino)-2-oxoethyl]amino]ethyl]amino]acetic acid;
2-[2-(bis(carboxymethyl)amino)ethyl-[2-[carboxymethyl-[2-(octadecylamino)-2-oxoethyl]amino]ethyl]amino]acetic acid;
5,8-bis(carboxymethyl)-2-(2-(methylamino)-2-oxoethyl)-10-oxo-2,5,8,11-tetraazadodecane-1-carboxylic acid;
5,8-bis(carboxymethyl)-2-(2-(hexylamino)-2-oxoethyl)-10-oxo-2,5,8,11-tetraazaheptadecane-1-carboxylic acid;
5,8-bis(carboxymethyl)-2-(2-(octylamino)-2-oxoethyl)-10-oxo-2,5,8,11-tetraazanonadecane-1-carboxylic acid;
5,8-bis(carboxymethyl)-2-(2-(decylamino)-2-oxoethyl)-10-oxo-2,5,8,11-tetraazahenicosane-1-carboxylic acid;
5,8-bis(carboxymethyl)-2-(2-(dodecylamino)-2-oxoethyl)-10-oxo-2,5,8,11-tetraazatricosane-1-carboxylic acid;
5,8-bis(carboxymethyl)-2-(2-(hexadecylamino)-2-oxoethyl)-10-oxo-2,5,8,11-tetraazaheptacosane-1-carboxylic acid;
5,8-bis(carboxymethyl)-2-(2-(octadecylamino)-2-oxoethyl)-10-oxo-2,5,8,11-tetraazanonacosane-1-carboxylic acid;
5,8-bis(carboxymethyl)-2-(2-(methylamino)-2-oxoethyl)-10-oxo-2,5,8,11-tetraazahenicosane-1-carboxylic acid;
5,8-bis(carboxymethyl)-2-(2-(decyloxy)-2-oxoethyl)-10-oxo-11-oxa-2,5,8-triazahenicosane-1-carboxylic acid;
2,2′-(17-(2-(2-aminoethylamino)-2-oxoethyl)-12,22-dioxo-11,14,17,20,23-pentaazatritriacontane-14,20-diyl)bis(N-(2-aminoethyl)acetamide);
2,2′-(17-(2-(2-(2-aminoethylamino)ethylamino)-2-oxoethyl)-12,22-dioxo-11,14,17,20,23-pentaazatritriacontane-14,20-diyl)bis(N-(2-(2-aminoethylamino)ethyl)acetamide);
2,2′-(17-(16-amino-2-oxo-7,10,13-trioxa-3-azahexadecyl)-12,22-dioxo-11,14,17,20,23-pentaazatritriacontane-14,20-diyl)bis(N-(3-(2-(2-(3-aminopropoxy)ethoxy)ethoxy)propyl)acetamide); and
2,2′-(12,22-dioxo-17-(15-oxo-2,5,8,11-tetraoxa-14-azahexadecan-16-yl)-11,14,17,20,23-pentaazatritriacontane-14,20-diyl)bis(N-(2,5,8,11-tetraoxamidecan-13-yl)acetamide).

6. A compound comprising the structure shown in Formula I: 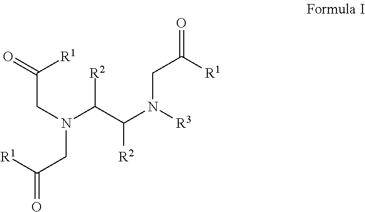
wherein
R¹ are independently, for each occurrence, selected from
—OR⁴, —O(CH₂)ₙOR⁴, —O(CH₂CH₂O)ₗR⁴, —O(CH₂CH₂NH)ₘR⁴, —O(CH₂)ₙNR⁴R⁵;
wherein
R⁴ and R⁵ are independently, for each occurrence, hydrogen or a substituted or unsubstituted C(1-22)alkyl having 1, 2, 3, 4, 5, 6, 7, 8, 9, 10, 11, 12, 13, 14, 15, 16, 17, 18, 19, 20, 21, or 22 carbon atoms or C(2-22)alkenyl having 2, 3, 4, 5, 6, 7, 8, 9, 10, 11, 12, 13, 14, 15, 16, 17, 18, 19, 20, 21, or 22 carbon atoms;
wherein R⁴ and R⁵ of —NR⁴R⁵ optionally form, together with the nitrogen atom of —NR⁴R⁵ to which they are attached, a saturated or unsaturated heterocyclic group optionally comprising one or more heteroatoms selected from oxygen, nitrogen and sulfur;
wherein one or more of the R¹ contains 4, 5, 6, 7, 8, 9, 10, 11, 12, 13, 14, 15, 16, 17, 18, 19, 20, 21, or 22 carbon atoms;
R² are independently, for each occurrence, selected from
hydrogen, C(1-6)alkyl, —(CH₂)ₙaryl, —(CH₂)ₙ(C₆H₄)R⁶, —(CH₂)ₙ(C₆H₄)(CH₂)ₙNR⁴R⁵;
wherein R⁶ is hydrogen, —OR⁴, —NR⁴R⁵; —O(CH₂)ₙOR⁴, —O(CH₂)ₙNR⁴R⁵, —(CH₂CH₂O)ₗR⁴; or —(CH₂CH₂NH)ₘR⁴;
R³ is selected from the group consisting of hydrogen, —(CH₂)ₙCOR¹, and 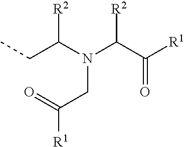
wherein
n is 1 to 22;
l is 1 to 50;
m is 1 to 50;
and salts thereof.

7. A compound comprising the structure shown in Formula I: 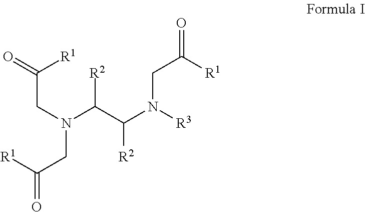
wherein
R¹ are independently, for each occurrence, selected from
a peptide comprising 2-50 amino acid residues;
wherein one or more of the R¹ contains 4, 5, 6, 7, 8, 9, 10, 11, 12, 13, 14, 15, 16, 17, 18, 19, 20, 21, or 22 carbon atoms;
R² are independently, for each occurrence, selected from
hydrogen, C(1-6)alkyl, —(CH₂)ₙaryl, —(CH₂)ₙ(C₆H₄)R⁶, —(CH₂)ₙ(C₆H₄)(CH₂)ₙNR⁴R⁵;
wherein R⁶ is hydrogen, —OR⁴, —NR⁴R⁵; —O(CH₂)ₙOR⁴, —O(CH₂)ₙNR⁴R⁵, —(CH₂CH₂O)ₗR⁴; or —(CH₂CH₂NH)ₘR⁴;
wherein R⁴ and R⁵ are independently, for each occurrence, hydrogen or a substituted or unsubstituted C(1-22)alkyl having 1, 2, 3, 4, 5, 6, 7, 8, 9, 10, 11, 12, 13, 14, 15, 16, 17, 18, 19, 20, 21, or 22 carbon atoms or C(2-22)alkenyl having 2, 3, 4, 5, 6, 7, 8, 9, 10, 11, 12, 13, 14, 15, 16, 17, 18, 19, 20, 21, or 22 carbon atoms;
wherein R⁴ and R⁵ of —NR⁴R⁵ optionally form, together with the nitrogen atom of —NR⁴R⁵ to which they are attached, a saturated or unsaturated heterocyclic group optionally comprising one or more heteroatoms selected from oxygen, nitrogen and sulfur;
R³ is selected from the group consisting of hydrogen, —(CH₂)ₙCOR¹, and 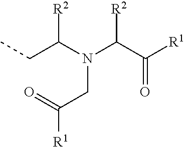
wherein
n is 1 to 22;
l is 1 to 50;
m is 1 to 50;
and salts thereof.

8. A compound comprising the structure shown in Formula III: 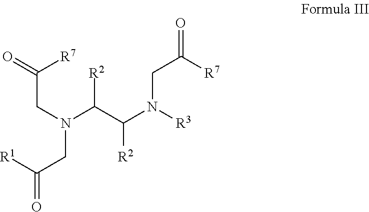
wherein
R¹ are independently, for each occurrence, selected from
—OR⁴, —O(CH₂)ₙOR⁴, —O(CH₂CH₂O)ₗR⁴, —O(CH₂CH₂NH)ₘR⁴, —O(CH₂)ₙNR⁴R⁵;
—NHR⁴, —NR⁴R⁵, —NH(CH₂CH₂O)ₗR⁴, —NH(CH₂CH₂NH)ₘR⁴, —NH(CH₂)ₙNR⁴R⁵, —NH(CH₂CH₂NH)ₘ(CH₂)ₙNR⁴R⁵, —NH(CH₂CH₂O)ₗ(CH₂)ₙNR⁴R⁵, —NH(CH₂CH₂O)ₗNR⁴R⁵; —NH(CH₂CH₂NH)ₘ(CH₂)ₙOR⁴;
R² are independently, for each occurrence, selected from
hydrogen, C(1-6)alkyl, —(CH₂)ₙaryl, —(CH₂)ₙ(C₆H₄)R⁶, —(CH₂)ₙ(C₆H₄)(CH₂)ₙNR⁴R⁵;
wherein R⁶ is hydrogen, —OR⁴, —NR⁴R⁵; —O(CH₂)ₙOR⁴, —O(CH₂)ₙNR⁴R⁵, —(CH₂CH₂O)ₗR⁴; or —(CH₂CH₂NH)ₘR⁴;
wherein R⁴ and R⁵ are independently, for each occurrence, hydrogen or a substituted or unsubstituted C(1-22)alkyl having 1, 2, 3, 4, 5, 6, 7, 8, 9, 10, 11, 12, 13, 14, 15, 16, 17, 18, 19, 20, 21, or 22 carbon atoms or C(2-22)alkenyl having 2, 3, 4, 5, 6, 7, 8, 9, 10, 11, 12, 13, 14, 15, 16, 17, 18, 19, 20, 21, or 22 carbon atoms;
wherein R⁴ and R⁵ of —NR⁴R⁵ optionally form, together with the nitrogen atom of —NR⁴R⁵ to which they are attached, a saturated or unsaturated heterocyclic group optionally comprising one or more heteroatoms selected from oxygen, nitrogen and sulfur;
R³ is selected from the group consisting of hydrogen, —(CH₂)ₙCOR¹, and 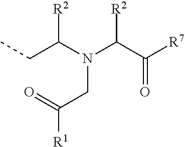
R⁷ is independently, for each occurrence, a D- or L-amino acid residue having the formula —NR^{N}—CR⁸R⁹—(C═O)—NR⁴R⁵—NR^{N}—CR⁸R⁹—(C═O)—OH, wherein
R⁸ is independently, for each occurrence, a substituted or unsubstituted side chain of an amino acid;
R⁹ is independently, for each occurrence, hydrogen, or an organic group consisting of carbon, oxygen, nitrogen, sulfur, and hydrogen atoms, and having from 1 to 20 carbon atoms, or C(1-5)alkyl, cycloalkyl, cycloalkylalkyl, C(3-5)alkenyl, C(3-5)alkynyl, C(1-5)alkanoyl, C(1-5)alkanoyloxy, C(1-5)alkoxy, C(1-5)alkoxy-C(1-5)alkyl, C(1-5)alkoxy-C(1-5)alkoxy, C(1-5)alkyl-amino-C(1-5)alkyl-, C(1-5)dialkyl-amino-C(1-5)alkyl-, nitro-C(1-5)alkyl, cyano-C(1-5)alkyl, aryl-C(1-5)alkyl, 4-biphenyl-C(1-5)alkyl, carboxyl, or hydroxyl,
R^{N} is independently, for each occurrence, hydrogen, or an organic group consisting of carbon, oxygen, nitrogen, sulfur, and hydrogen atoms, and having from 1 to 20 carbon atoms, or C(1-5)alkyl, cycloalkyl, cycloalkylalkyl, C(3-5)alkenyl, C(3-5)alkynyl, C(1-5)alkanoyl, C(1-5)alkanoyloxy, C(1-5)alkoxy, C(1-5)alkoxy-C(1-5)alkyl, C(1-5)alkoxy-C(1-5)alkoxy, C(1-5)alkyl-amino-C(1-5)alkyl-, C(1-5)dialkyl-amino-C(1-5)alkyl-, nitro-C(1-5)alkyl, cyano-C(1-5)alkyl, aryl-C(1-5)alkyl, 4-biphenyl-C(1-5)alkyl, carboxyl, or hydroxyl,
wherein
n is 1 to 22;
l is 1 to 50;
m is 1 to 50;
and salts thereof.

9. The compound of claim 8, wherein the side chain of the amino acid is that of arginine, homoarginine, norarginine, nor-norarginine, ornithine, lysine, homolysine, histidine, 1-methylhistidine, pyridylalanine, asparagine, N-ethylasparagine, glutamine, 4-aminophenylalanine, the N-methylated versions thereof, and side chain modified derivatives thereof.

10. The compound of claim 1, wherein the nucleic acid is attached via a linker.

11. The compound of claim 1, wherein the nucleic acid is an RNA molecule

12. The compound of claim 1, wherein the nucleic acid is an RNAi agent.

13. A composition comprising a compound of claim 1 and a drug.

14. A composition comprising a compound of claim 1 and a peptide drug.

15. A composition comprising a compound of claim 1 and a nucleic acid.

16. A composition comprising a compound of claim 1 and an RNAi agent.

17. A composition comprising a compound of claim 1, an interfering RNA agent, and a lipid.

18. A method for treating the signs and symptoms of rheumatoid arthritis, liver disease, heart disease, viral disease, hepatitis, influenza, cancer, liver cancer, or bladder cancer in a subject comprising administering to the subject in need a therapeutic amount of a composition of claim 13.

19. A method for delivering a nucleic acid to a cell comprising contacting the cell with a composition according to claim 15.

20. A method for inhibiting expression of a gene in a mammal comprising administering to the mammal a composition according to claim 15.
